(19) United States
(12) Patent Application Publication
(10) Pub. No.: US 2013/0177972 A1
(43) Pub. Date: Jul. 11, 2013

(54) BIOREACTORS, SYSTEMS, AND METHODS FOR PRODUCING AND/OR ANALYZING ORGANS

(71) Applicants: David Green, Dover, MA (US); Joseph Consiglio, Holliston, MA (US); Ron Sostek, Newton, MA (US)

(72) Inventors: David Green, Dover, MA (US); Joseph Consiglio, Holliston, MA (US); Ron Sostek, Newton, MA (US)

(21) Appl. No.: 13/510,421

(22) PCT Filed: Nov. 17, 2010
(86) PCT No.: PCT/US10/02995

§ 371 Date: Mar. 27, 2013

Related U.S. Application Data
(60) Provisional application No. 61/262,130, filed on Nov. 17, 2009.
(60) Provisional application No. 61/298,393, filed on Jan. 26, 2010.

(51) Int. Cl. C12M 3/00; G06K 19/06; G06K 19/067

(52) U.S. Cl. CPC C12M 21/08; G06K 19/067; G06K 19/06187; G06K 19/06009
USPC 435/288.7; 435/289.1; 435/287.1; 235/487; 235/492; 235/493; 235/494

(57) ABSTRACT

Articles and methods for growing or analyzing tissues and organs using bioreactors or other devices and components are provided. In some embodiments, a bioreactor is configured to provide a growth chamber having one or more inlets, outlets, sensors, organ attachment sites, and/or organ identifiers.

### RELATED APPLICATIONS

The present application claims the benefit under 35 U.S.C. 119(e) of the filing dates of U.S. Provisional Patent Application 61/262,130 filed on Nov. 17, 2009 and U.S. Provisional Patent Application 61/298,393 filed on Jan. 26, 2010, the disclosures of which are incorporated herein in their entirety.

### FIELD OF INVENTION

The present invention relates generally to articles and methods for growing and analyzing tissues and organs, and, more specifically, to growing and analyzing tissues and organs for transplantation using bioreactors or other devices and components.

### BACKGROUND

A major goal in the field of tissue and organ regeneration is to be able to grow body parts that can be used for transplantation into individuals in order to treat different medical conditions that are associated with organ or tissue disease, aging, failure, and/or injury. The growth of artificial organs and tissues in vitro has been studied for many years. Significant progress has been made in the research context towards an understanding of the biological and physiological processes that are associated with the regeneration of tissues and organs from cellular preparations. Examples of several basic organ models or partial organs have been grown in vitro, including models of lung and liver. However, there still are significant challenges to developing therapeutic programs for growing organs from cells and obtaining fully developed and functional organs that are suitable for implantation into a patient for therapeutic purposes.

### SUMMARY OF THE INVENTION

The present invention relates to systems, devices, and methods that can be used to improve the process of organ growth, transport, and transplantation. In different embodiments, aspects of the invention are useful to identify growth conditions and environmental cues that improve the efficiency and/or reproducibility of organ or tissue growth; monitor and modulate organ growth in response to experimentally identified conditions and/or conditions that mimic a natural growth environment; evaluate organ or tissue growth to determine suitability for transplantation; provide safety features to monitor and/or control sterility, and/or to manage the process of matching an organ or tissue with an intended recipient (e.g., by monitoring and/or tracking the source or identity of the cells that were introduced into the reactor for regeneration); and/or to provide structural or functional features on a substitute tissue or organ that are useful during the transplantation procedure to help make structural and functional connections to the recipient body.

In some embodiments, aspects of the invention relate to systems that provide a suitable environment for performing one or more growth, transportation, and/or storage functions associated with an organ or tissue growth program. According to aspects of the invention, organ development (e.g., based on speed and/or organ quality) may be significantly influenced (and improved in some cases) by changing the growth conditions during development. Accordingly, aspects of the invention relate to devices and methods that can be used to change (e.g., up or down) organ growth conditions once or more during development in response to one or more parameters or cues described herein (e.g., based on predetermined time intervals, levels of one or more variables, images, etc., or combinations thereof as described herein). In some embodiments, a modular system is provided with all or most of the components and materials that are to be used during growth of one or more organs within the system. Individual components may be removed as the organ(s) progress through growth and the resulting product may be an organ in a chamber that is suitable for use as a storage or transportation unit. These and other aspects are described in more detail herein and are useful both to optimize the growth of individual organs, and also to manage a large scale process of organ growth and development that involves tracking and producing different organs.

In some embodiments, the present invention relates generally to systems and methods for growing and analyzing two-dimensional (2D) and three-dimensional (3D) tissues, tissue complexes and organs for transplantation and other uses. In some embodiments, aspects of the invention relate to methods and systems for introducing cells into bioreactors in order to grow substitute organs or tissues that can be used to supplement or replace one or more functional or structural properties of an organ or tissue that is failing (e.g., due to age, injury, disease, etc., or any combination thereof) in a subject. In some embodiments, aspects of the invention are directed to providing substitute organs and tissues that have appropriate structural and/or functional properties (not merely viability) upon transplantation into a subject. In some embodiments, aspects of the invention are directed to providing substitute organs or tissues that are adapted for transplantation into a subject. In some embodiments, aspects of the invention are directed to maintaining correct organ or tissue identity so that a substitute organ or tissue is correctly matched to a recipient. One or more of these features may be embodied in bioreactors, related systems and components, methods, and/or databases as described in more detail herein. In some embodiments, aspects of the invention provide bioreactors and related methods and systems that are designed to allow organ or tissue growth conditions to be monitored and altered during growth and development. In some embodiments, aspects of the invention include features for growing substitute organs or tissues that have structural or functional properties adapted for surgical manipulation. In some embodiments, aspects of the invention include features for tracking and protecting the identity of a substitute organ or tissue during growth, development, storage, and/or transport.

In some embodiments, methods, devices, and systems are provided for introducing cells into a bioreactor to grow substitute tissues or organs in a bioreactor designed for 2D and/or 3D production while analyzing cells, tissues and organs for viability, physiological functionality and/or structural integrity. In some embodiments, sensors, specialized bioreactor devices, specific procedures, databases, and/or other devices and components may be used to assess and produce suitable (e.g., normal or otherwise acceptable) substitute tissue or organs. A suitable substitute tissue may be a tissue or tissue complex that has properties that are useful for human, animal or cellular transplantation. Similarly, a suitable substitute organ may be an organ, portion of an organ, or appropriate artificial cellular structure that can provide one or more useful physiological organ functions for human, animal, or cellular transplantation.

In some embodiments, methods, devices, and systems are provided for maximizing proper growth of cells in a bioreactor by defining one or more (e.g., 2, 3, 4, 5, 6, 7, 8, 9, 10, or more) growth phases (each characterized by different growth conditions and/or different growth and functional properties) and assessing growth relative to acceptable physiological, metabolic, histological, structural, and/or mechanical variables. In some embodiments, the health and development status of a regenerated organ or tissue can be evaluated in order to produce a successfully characterized regenerated organ or tissue and/or in order to screen out abnormally regenerated organs or tissues. In some embodiments, methods are based on the recognition of the interrelationship of cues necessary for growing and assessing an organ that is healthy when compared to a normal organ of the same type. In some embodiments, methods, devices, and systems described herein monitor and/or manipulate variables relating to one or more of the following non-limiting parameters: spatial, physiological, metabolic, mechanical, chemical, histological, and/or structural inter-relationships between cells and organs in order to produce an appropriately functional substitute organ or tissue. In some embodiments, these relationships are measured and analyzed during and/or after the growth process to assure that development occurred in the correct sequence, and resulted in a substitute tissue, tissue complex or organ that has appropriate physiological properties. It should be appreciated that while the appropriate properties may be the normal or natural physiological properties for a particular organ or tissue, they are not required to be normal or natural if they provide sufficient function or structure to perform a desired role (e.g., assist or replace a defective tissue or organ).

In some embodiments, the tissues or organs being grown and/or analyzed are in vivo. In other embodiments the tissues or organs being grown and/or analyzed are ex vivo. In certain embodiments, the organs are substitute organs (e.g., regenerated organs, entire organs, organ portions, smaller organs that perform one or a subset of functions of an organ, artificial organs, cellular organelles, etc., or any combination thereof) that may be implanted into a subject (e.g., a human patient, an animal, or any other suitable recipient). In some embodiments, the articles and methods can be used to form biocompatible structures for research. In some embodiments, the articles and methods can be used to form biocompatible structures for tissue engineering and organ replacement. In some embodiments, the biocompatible structures may be surgically implanted in a recipient. In some embodiments, the biocompatible structures may be maintained in a device that can be connected to a subject to provide one or more missing functions. For example, a kidney substitute may be maintained outside a subject, within a device that can be connected to a subject, in order to perform one or more dialysis functions. Similarly, other organ functions (e.g., liver, pancreatic, and other organ functions) may be provided by a substitute organ that is not surgically implanted in a subject, but that is maintained in a device that can be connected to the subject (e.g., on a permanent or temporary basis). Articles and methods for detecting a condition of a tissue or organ of interest are also provided. The subject matter of the present invention involves, in some cases, interrelated products, alternative solutions to a particular problem, and/or a plurality of different uses of one or more systems and/or articles.

In some embodiments, aspects of the invention relate to a bioreactor comprising a chamber. The chamber may include a first inlet port and a first outlet port in fluid communication with the chamber. In some embodiments, the chamber may have two or more inlet ports, two or more outlet ports, or any combination thereof as described in more detail herein. It should be appreciated that in some embodiments, one or more ports may act both as inlet and outlet ports by being configured to allow fluid flow into the chamber or out of the chamber. The control of fluid flow into or out of the chamber may be determined by a pump, a valve, a difference in pressure, or any other mechanism or factor that controls the direction of fluid flow.

The chamber may include an organ support structure. In some embodiments, the organ support structure is connected to a scale, strain gauge, or other electrical or mechanical sensor for measuring the weight of the organ. In some embodiments, the organ support structure comprises a platform for support. In some embodiments, the platform is shaped to receive the lower portion of an organ. In some embodiments, the support structure may comprise one or more other organs (e.g., either natural or artificial), portions thereof, artificial objects, or any combination thereof that can be used to simulate natural conditions (e.g., the natural environment of the organ during growth, development, or function, for example, an environment that simulates the natural spatial relationship of the organ being grown relative to an adjacent or close organ or other defined object). It should be appreciated that the support structure may simulate a natural environment during a particular defined natural physiological period of time (e.g., a particular stage of development). In some embodiments, the support structure may be changed (e.g., with respect to size; shape; number, size, shape, and relative position of adjacent organs, portions thereof, or other objects). It should be appreciated that the spatial relationship of the organ being grown relative to adjacent organs, organ portions, or other organs, may include a relative distance and/or a relative three-dimensional configuration. In some embodiments, one or more spatial relationships provide cues for development and growth (e.g., secondary growth cues) in organ development. In some embodiments, actual tissue maybe added to the reactor, for example, to stimulate the organ being grown, or to inhibit or activate a secondary growth pattern.

In some embodiments, the organ support structure comprises a first support member. In some embodiments, the organ support structure comprises a first tubular connector adapted for attaching a vascular structure. In some embodiments, the first tubular connector is cylindrical. However, any suitable shape or configuration of a tubular connector may be used. In some embodiments, the first tubular connector comprises a flexible flange. In some embodiments, the first tubular connector comprises an elastic flange. In some embodiments, the first tubular connector comprises an expandable flange. In some embodiments, the expandable flange is remotely controlled. In some embodiments, the first tubular connector comprises a tapered end. In some embodiments, the first tubular connector comprises a flared end. In some embodiments, the organ support structure is in a fixed position relative to the chamber. In some embodiments, the organ support structure can rotate around a first axis that is in a fixed position relative to the chamber. In some embodiments, the organ support structure can rotate around a second axis that is in a fixed position relative to the chamber. In some embodiments, a bioreactor comprises a first sensor responsive to temperature, oxygen, carbon dioxide, pH, lactate, or glucose levels. In some embodiments, two or more sensors, each responsive to temperature, oxygen, carbon dioxide, pH, lactate, or glucose levels are used. In some embodiments, at least one sensor is connected to a readout via an optical cable. In some embodiments, the optical cable is connected through a sterile conduit in a wall of the chamber. In some embodiments, at least one sensor is connected to a wireless transmitter housed within the chamber. In some embodiments, an optical sensor is located inside the container and is aligned with an optical conduit that is located on the outside of the container. In such embodiments, the wall of the container, or at least a portion of the wall of the container (e.g., the portion across which the detector and conduit are aligned) is transparent to the optical signal (e.g., sufficiently transparent to allow transmission of an optical signal of interest).

It should be appreciated that one or more organ or tissue regions may be grown over a structure described herein (e.g., a flange or other support or connector structure). In some embodiments, growth of a portion of an organ or tissue may be directed by applying a scaffold (with or without growth factors) over the structure and contacting the scaffold with suitable cells under growth conditions.

In some embodiments, a bioreactor comprises a first sensor responsive to temperature, oxygen, carbon dioxide, pH, lactate, or glucose levels. In some embodiments, two or more sensors, each responsive to temperature, oxygen, carbon dioxide, pH, lactate, or glucose levels. In some embodiments, at least one sensor is connected to a readout via an optical cable.

In some embodiments, the bioreactor further comprises an electrical outlet housed within the chamber. In some embodiments, the electrical outlet is connected to a power cord that passes through a sterile conduit in a wall of the chamber. In some embodiments, the chamber wall comprises a sterile access port. In some embodiments, the chamber wall comprises an observation area that is transparent to infrared, UV, visible light and/or other forms of radiation described herein. In some embodiments, the chamber wall comprises a translucent portion.

In some embodiments, the translucent portion comprises polysulfone or other sterilizable material that is transparent to visible and/or infrared light for inspection or analysis of the growing material (e.g., tissue or organ or portion thereof). In some embodiments, the chamber comprises a wall comprising a flexible portion. In some embodiments, the chamber comprises a wall comprising a section of elastic material. In some embodiments, the bioreactor further comprises a second inlet port in fluid connection with a tubular structure adapted for attachment to a vascular structure. In some embodiments, the bioreactor further comprises a second outlet port in fluid connection with a tubular structure adapted for attachment to a vascular structure. In some embodiments, the bioreactor further comprises a pump. In some embodiments, the pump is connected to the first inlet and the first outlet port. In some embodiments, the pump is connected to the second inlet and the second outlet port. In some embodiments, the bioreactor comprises a first stimulatory means. In some embodiments, the stimulatory means can administer an electrical challenge to a substitute organ attached to the organ support structure. In some embodiments, the bioreactor further comprises a sensor capable of detecting a response to the electrical challenge. In some embodiments, the stimulatory means can administer a chemical challenge to a substitute organ attached to the organ support structure. In some embodiments, the bioreactor further comprises a sensor capable of detecting a response to the chemical challenge. In some embodiments, the stimulatory means can administer a physical challenge to a substitute organ attached to the organ support structure. In some embodiments, the bioreactor further comprises a sensor capable of detecting a response to the physical challenge. In some embodiments, the stimulatory means is connected to one or more afferent vessels of a substitute organ attached to the organ support structure. In some embodiments, the sensor is connected to one or more efferent vessels of a substitute organ attached to the organ support structure. In some embodiments, the challenge represents a physiological parameter selected from the group consisting of blood pressure, pH, oxygen, toxin, metabolite, airflow, substrate, hormone, and any combination thereof. In some embodiments, the response is a level of a physiological parameter selected from the group consisting of blood pressure, pH, oxygen, toxin, metabolite, and any combination thereof. In some embodiments, a bioreactor comprises a 2-dimensional or 3-dimensional array of sensors to determine the size, shape, weight, tensile strength, blood vessel strength, or strength of attachment to a substrate of a substitute organ attached to the organ support structure. In some embodiments, one or more sensors or pairs of sensors may be configured to detect and/or measure different types of force exerted by a substitute organ on a support structure. Examples of forces that can be measured include tension, pressure, torsion (e.g., using a torque sensor), or any combination thereof. It should be appreciated that any of these forces can be evaluated between two or more regions of the substitute organ by using appropriate sensors or pairs of sensors. In some embodiments, pressure with an organ may be measured using a pressure sensor that is attached to a conduit in fluid communication with the organ (e.g., with one or more blood vessels of the organ).

Accordingly, one or more mechanical, magnetic, electrical, optical, imaging, chemical, or other sensor(s), or any combination thereof may be used in connection with a bioreactor described herein. In some embodiments, one or more sensors may be attached to or embedded in a wall of a device as described in more detail herein. However, in some embodiments, a device is configured to allow one or more external sensors to detect and/or measure signal(s) generated within a reactor.

In some embodiments, suitable electro/mechanical and/or imaging techniques may be used to determine (e.g., measure) the chemical, physical, and/or physiological state of the substitute organ, of the bioreactor internal environment and condition, or a combination thereof.

In some embodiments, aspects of the invention provide information that can be used to direct and optimize growth of a substitute organ. The information may include one or more different growth conditions for different growth phases. This information may be based on i) natural growth conditions and natural changes in growth conditions that are observed as an organ develops and matures in vivo; ii) experimental growth conditions or changes in growth condition that have been determined to be helpful for the growth and development of a substitute organ; or any combination of i) and ii). It should be appreciated that the growth conditions may include temperature, chemical, mechanical, nutritional, electromagnetic, and/or other factors, or combinations thereof. It also should be appreciated that optimal growth conditions or changes in growth conditions may be different for different organs, tissues, tissue complexes, and also may be species or gender specific. In addition, factors such as size, age, and other physiological parameters may affect the growth conditions or changes in growth conditions that are used.

In some embodiments, aspects of the invention provide information that can be used to evaluate the status or health of a substitute organ during growth. Appropriate growth patterns of substitute organs can be determined and then used as a reference for determining whether one or more substitute organs being grown are developing appropriately. In some embodiments, one or more parameters are evaluated at different times points (e.g., 2, 3, 4, 5, 5-10, 10-15, 15-20, or more) during the growth and development of the substitute organ and compared to the reference information. It should be appreciated that any suitable time interval may be used (e.g., measurements may be hourly, daily, weekly, or more or less frequent, depending on the growth and/or storage stage). In some embodiments, if the parameters are within acceptable ranges of the reference information, the organ is determined to be acceptable and growth and development are continued until a further evaluation at the next time point. However, if at any time point one or more parameters are determined to be outside acceptable ranges of the reference information, a decision or intervention may be required. In some embodiments, growth of the organ may be terminated if the organ is determined to be unacceptable based on the analysis. In some embodiments, growth conditions may be changed in order to correct one or more growth deficiencies that are identified.

It should be appreciated that in some embodiments, the information may provide a reference for unhealthy or unacceptable growth rather than a reference for healthy or acceptable growth. Accordingly, if one or more parameter values are determined to be similar to reference values indicative of a problem, a decision may be made to either terminate the organ or to intervene to correct potential deficiencies.

It should be appreciated that any suitable information described herein may be used to determine appropriate growth conditions, evaluate whether a substitute organ is growing normally (e.g., is healthy or physiologically acceptable), and/or is growing abnormally (e.g., shows signs of inappropriate growth or function). The information may be stored on a database and accessed to be used for programming growth conditions and/or comparing substitute organ growth to a reference at one or more time points.

For example, in some embodiments information may be compared to a database of normal values, normal developmental phase images or mechanical, histological, electrical, and/or chemical values which can be evaluated using multi-variant approaches to assess the development of a organ. The information can be used to determine the degree of fit relative to reference points or ranges that can be included in a database to represent good and/or bad values that can be used subsequently to assess the proper and/or improper development of substitute organs in a bioreactor. It should be appreciated that a database may include one or more of the following non-limiting types of information: species, organ, date of tissue, source of tissue, organ type, tissue type, scaffold type, temperatures of incubation, infrared and/or visible confluence images, O2, CO2, pH, lactate, glucose, creatine, start date, projected end date, or other information, or any combination thereof. In some embodiments, images and data can be updated with every new production cycle and the data can be compared to the new production goals to determine fit relative to good models and for maximizing yield time.

In some embodiments, each of the chamber, inlet port, outlet port, and organ support structure contains only material that is compatible for use with an MRI, CAT, PET, X-ray analysis, or ultrasound device, or other devices, detectors, and detection methods described herein. In some embodiments, the material is non-metallic. In some embodiments, the material is non-paramagnetic. In some embodiments, the material is Lucite, glass or other compatible material. In some embodiments, the chamber, inlet port, and outlet port are fabricated of the same material. In some embodiments, the material of the chamber is different from the material of the inlet port or the outlet port. In some embodiments, the bioreactor further comprises a substitute organ attached to the organ support structure. In some embodiments, the bioreactor further comprises a scaffold attached to the organ support structure. In some embodiments, the scaffold is a decellularized organ scaffold. In some embodiments, the scaffold is a biopolymer generated scaffold. In some embodiments, the substitute organ is a substitute solid organ. In some embodiments, the substitute solid organ is a substitute lung, liver, kidney, heart, or pancreas. In some embodiments, the substitute organ comprises a prevascularized structure. In some embodiments, the bioreactor further comprises a support member for connecting a prevascularized structure. In some embodiments, the bioreactor further comprises a manifold for connecting two or more prevascularized structures.

In some embodiments, the bioreactor comprises a first tag for identifying, tracking, or confirming the origin of a substitute organ attached to the organ support structure. In some embodiments, the first tag is an electronic tag, a magnetic tag, an RFID tag, a barcode, or any combination thereof.

In some embodiments, the bioreactor comprises a means for removing cells from the chamber to identify, track, identify, or confirm the origin of a substitute organ attached to the organ support structure.

In some embodiments, the bioreactor comprises an injector for injecting material into a substitute organ attached to the organ support structure. In some embodiments, a bioreactor comprises a biopsy device for removing material from a substitute organ attached to the organ support structure. In some embodiments, a bioreactor is connected to a pump via one or more conduits, wherein each of the chamber, pump, and one or more conduits are of material that is compatible with use with an MRI, CAT, PET, X-ray analysis, or ultrasound device, or other devices, detectors, and detection methods described herein. In some embodiments, the material of each of the chamber, pump, and one or more conduits is non-metallic. In some embodiments, the material of each of the chamber, pump, and one or more conduits is non-paramagnetic. In some embodiments, the material of each of the chamber, pump, and one or more conduits, is the same. In some embodiments, the material of the chamber is different from the material of the pump, the one or more conduits, or both.

It should be appreciated that any of these devices or components may be sterilized using an appropriate technique.

In some embodiments, aspects of the invention relate to a single core reactor that can be used for decellularizing scaffolds, regenerating tissues and/or organs, storing the tissues and/or organs, and/or transporting the tissue and/or organs to a medical or surgical location where the tissue and/or organ is removed from the reactor and implanted into a subject. Accordingly, all or a portion of the reactor may be disposable.

However, in some embodiments, all or a portion of the reactor may be re-usable. In some embodiments, a multistage reactor system is provided that is modular and includes several components that may be used at different stages during development of the substitute organ or tissue (e.g., during the decellularization, recellularization, growth, storage, and/or transport stages). The different components may include different connectors, support structures, sensors, controllers, mechanical devices, storage volumes, buffers, power supplies, temperature regulators (e.g., to heat or cool solutions) etc., or any combination thereof. In some embodiments, a reactor chamber may include several zones that are used for different processes. As the development of the organ progresses, one or more components may be disconnected and/or removed (e.g., discarded) after they are used. In some embodiments, as organ development progresses, one or more zones of the chamber may be disconnected and/or removed (e.g., discarded) after they are used. It should be appreciated that sterile mechanisms and procedures should be used during the process of disconnecting a component or a zone of a reactor in order to maintain the sterility of the developing organ or tissue. In some embodiments, sterile connectors with valves or other mechanisms for maintaining a sealed sterile chamber may be used for disconnecting the components or reactor zones.

In some embodiments, aspects of the invention relate to a kit comprising a first tag to be attached to an organ recipient and a second tag to be attached to a device (e.g., bioreactor) or any component thereof described herein or to a substitute organ within said bioreactor. In some embodiments, the first tag is a bracelet. In some embodiments, the first and second tags are independently selected from an electronic tag, a magnetic tag, an RFID tag, a barcode, or any combination thereof. In some embodiments, the first and second identifier tags are identical. In some embodiments, the first and second identifier tags are different. In some embodiments, the first and second identifier tags are complementary tags that generate a specific signal when matched. In some embodiments, the first and second identifier tags are complementary electronic tags, magnetic tags, RFID tags, barcodes, or any combination thereof. In some embodiments, a kit comprises one or more components for performing an assay to determine a DNA match, an HLA match, a unique protein match, or a combination thereof. In some embodiments, the kit further comprises growth reagents and stimulatory reagents, wherein the growth reagents are sufficient to support growth of a substitute organ in a bioreactor and wherein the stimulatory reagents are suitable to challenge one or more physiological responses of the substitute organ. In some embodiments, a bioreactor chamber has a volume of between 20 cc and 20,000 cc. In some embodiments, the chamber volume is between 500 cc and 1,000 cc. In some embodiments, the chamber volume is between 1,000 cc and 10,000 cc. In some embodiments, the chamber volume is between 10,000 cc and 20,000 cc. However, any suitable chamber size (e.g., including larger or smaller chambers) may be used to accommodate the organ being prepared, as aspects of the invention are not limited in this respect.

In some embodiments, the bioreactor is sealed. In some embodiments, the bioreactor is sterile.

It should be appreciated that any of the bioreactors described herein may be used to grow a substitute organ for research and/or for clinical transplantation. As used herein a substitute organ may be a complete organ or a partial organ that has at least one or more physiological functions of an organ. For example, a partial kidney may produce erythropoietin (EPO) but not filter the blood. Accordingly, in some embodiments, a partial organ may have one or more secretory functions of an organ (e.g., it produces and/or secretes one or more compounds, for example a hormone, that a natural organ produces and/or secretes). However, a partial organ may have one or more other properties of an organ as the invention is not limited in this respect. For example, a partial organ may perform one or more detoxification properties of a liver. In some embodiments, a substitute organ may be grown on a scaffold (e.g., a natural or synthetic scaffold). In some embodiments, a substitute organ may be based on a decellularized scaffold of a first organ that is recellularized with cells of the same organ type to reconstitute similar organ functions. In some embodiments, a substitute organ may be based on a decellularized scaffold of a first organ that is recellularized with cells from a different second organ. For example, a first organ (e.g., kidney) may be decellularized and the resulting scaffold may be recellularized with cells that have one or more properties (e.g., secretory properties) of a second organ (e.g., liver). In some embodiments, a kidney may be a useful first organ to use to produce a scaffold since a subject has two kidneys and one of them may be removed to produce a scaffold for a substitute organ in the same subject.

Accordingly, in some embodiments a bioreactor comprises a scaffold upon which cells can be seeded. In some embodiments, the scaffold is a matrix. In some embodiments, the scaffold comprises an axis. In some embodiments, the bioreactor comprises a chamber that contains a support that is capable of rotating around one or more axes. A bioreactor may be provided without a scaffold and include one or more structures to which a scaffold may be attached. A bioreactor may include one or more support structures to support the weight of a growing organ. It should be appreciated that a structure to which a scaffold is attached also may support the weight of a growing organ. However, one or more scaffold and organ support structures may be different as described herein. In some embodiments, a substitute organ may be grown without using a scaffold, but the weight of the organ could be supported by one or more structures within a bioreactor. It should be appreciated that one or a plurality of attachment points for the scaffold (e.g., 2-5, 5-10, or more) may be provided in a bioreactor.

However, as described in more detail herein, a substitute organ can be produced without a scaffold. For example, in some embodiments, a substitute organ can be based on a micro-channel containing device having tissues that are grown to produce particular chemicals or to perform particular detoxification steps that can mimic one or more functions of a natural organ. It should be appreciated that these can be single or multiplayer devices with no scaffold that act like an organ when transplanted into a host.

In some embodiments, aspects of the invention may be used to determine the growth parameters, and/or organ properties that can be monitored and/or evaluated to determine when a substitute organ is ready for transplantation. This information can be stored in a database and used as described in more detail herein. Once an organ is ready, it may be stored (under the same or different conditions) until the recipient is ready for the implantation procedure.

In another set of embodiments, a method of assessing a condition of at least one portion of a tissue or organ of interest is provided. The method includes positioning an infrared detector near a tissue or organ of interest and detecting infrared radiation emanating from at least one portion of the tissue or organ. The method also includes analyzing the detected infrared radiation and generating data corresponding to the at least one portion of the tissue or organ. A condition of the at least one portion of the tissue or organ can be determined based, at least in part, on the generated data.

In another set of embodiments, a head-mounted device is provided. The head-mounted device includes at least two detectors that allows an orthogonal viewing ability, a WYSIWYG optical viewing system, and a real-time auto-focus ability. The head-mounted device also includes an image stabilization controller, a microscope comprising at least a 10×, at least a 15×, at least a 20×, at least a 50×, at least a 100×, at least a 250×, or at least a 500× magnification ability, and a binocular telescope. A controller may be operatively associated with the head-mounted device. In some cases, the controller is controlled via a foot pedal. In other cases, the controller is controlled via voice control. The head-mounted device may optionally include a source of radiation that can be emitted from the device. For example, the source of radiation may emit radiation in the infrared, near-infrared, visible, or ultraviolet range. In some embodiments, each of the at least two detectors is adapted to detect one or more of absorbance, transmission, reflectance, infrared radiation, radiation from the visible range, vibrational radiation, pressure, fluorescence radiation, Raman radiation, and/or temperature. In some cases, the head-mounted device includes detectors adapted and arranged to detect at least two, at least three, or at least four, or at least five of absorbance, transmission, reflectance, infrared radiation, radiation from the visible range, vibrational radiation, pressure, fluorescence radiation, Raman radiation, and/or temperature. The head-mounted device may also be adapted and arranged to analyze data collected from the two or more detectors. In some cases, the head-mounted device is adapted and arranged to generate at least two images corresponding to the data collected from the two or more detectors. The head-mounted device may be adapted and arranged to superimpose the at least two images. In some embodiments, the head-mounted device includes a spectral filter. The head-mounted device may also have other characteristics and components described herein. Information from a head-mounted device may be combined with other information obtained from sensors as described herein and used to determine optimal growth conditions and patterns, monitor the progress of tissue or organ development based on known or experimental determined models, and/or determine when tissues or organs are either suitable for transplantation and/or should be discarded as abnormal or unsuitable for further use.

In some embodiments, aspects of the invention provide sterilizable multistage systems and devices that include a sterile chamber having one or more zones that can be isolated and/or disconnected, one or more components (e.g.; stimulators, sensors, storage components), and related power supplies, pumps, displays, controllers, etc., each of which can be disconnected and/or discarded as the organ or tissue development proceeds.

It should be appreciated that methods and devices described herein (e.g., for identifying optimal development conditions, identifying or using cues for changes in conditions, identifying or using signals for evaluating the condition of a tissue or organ, or any combination thereof) may be used for tissue or organ development, and also for organelle development (e.g., to regenerate a cellular organelle such as a mitochondria that also can be replaced in a cellular context).

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic diagram showing a side view of a bioreactor according to a first set of embodiments;

FIG. 1B is a schematic diagram showing a side view of a bioreactor according to a second set of embodiments;

FIG. 2 shows a schematic diagram of a system according to one set of embodiments;

FIGS. 3A and 3B show schematic diagrams of an organ support structure according to two sets of embodiments;

FIG. 3C shows a schematic diagram of a bioreactor comprising a mechanical sensor (e.g., a torque sensor) to detect and monitor mechanical properties of a substitute organ during growth;

FIG. 4 illustrates a non-limiting example of a heart that is being evaluated to identify its pattern of spatial vibrational and heat distributions according to one set of embodiments; and

FIG. 5 is a schematic diagramming showing a non-limiting example of a cylindrical rolling member according to one set of embodiments.

### DESCRIPTION

The present invention relates to systems, devices, and methods that can be used to improve the process of organ growth, transport, and transplantation. In different embodiments, aspects of the invention provide reactor chambers with one or more sensors and related components that can be used to provide feedback on and/or modulate (e.g., automatically and/or manually) growth conditions within the reactor. Aspects of the invention are useful to identify growth conditions and environmental cues that improve the efficiency and/or reproducibility of organ or tissue growth; monitor and modulate organ growth in response to experimentally identified conditions and/or conditions that mimic a natural growth environment; evaluate organ or tissue growth to determine suitability for transplantation; provide safety features to monitor and/or control sterility, and/or to manage the process of matching an organ or tissue with an intended recipient; and/or to provide structural or functional features on a substitute tissue or organ that are useful during the transplantation procedure to help make structural and functional connections to the recipient body.

In some embodiments, the present invention relates generally to articles and methods for growing and analyzing two-dimensional (2D) and three-dimensional (3D) tissues, tissue complexes and organs. Certain embodiments are particularly useful for providing appropriate cues and stimuli to grow and/or store substitute organs and tissues that have appropriate physiological properties (in addition to mere viability) for subsequent transplantation into a recipient (e.g., in order to supplement one or more deficient physiological functions). It should be appreciated that aspects of the invention may be used for any applications where substitute organs or tissues may be useful, for example, in therapeutic, research, and/or manufacturing contexts.

In some embodiments, methods, devices, and systems are provided for monitoring and/or adjusting growth conditions in a bioreactor. In some embodiments, optimal growth conditions may involve one or more changes in conditions during a growth period in order to promote appropriate organ or tissue growth and development. In contrast to existing techniques that involve growing organs or portions thereof using a particular set of growth conditions, certain methods of the invention may include cycling through a series of conditions during growth and development of the substitute organ or tissue. In some embodiments, one or more cycles may be designed to mimic natural changes in growth conditions. However, artificial changes in growth conditions may be incorporated into a technique, for example, if the changes have been shown experimentally to be good for promoting particular organs or tissue types.

Accordingly, in some embodiments, aspects of the invention relate to systems and methods for growing and analyzing cells, tissues and organs for viability, physiological functionality and structural integrity. In some embodiments, specialized bioreactors, sensors, controllers, systems (e.g., automated feedback systems, and/or systems allowing manual control and/or override of automated processes), and related databases may be used to determine and implement growth parameters that have been developed for particular substitute organs and tissues.

Methods and devices described herein may be useful for implementing substitute tissue and organ growth in a production context as opposed to a small-scale research context. In some embodiments, aspects of the invention address transport, storage, tracking, sterility, functional screening, and other challenges associated with large scale substitute organ or tissue production.

In some embodiments, the tissues or organs being grown and/or analyzed are in vivo. In other embodiments the tissues or organs being grown and/or analyzed are ex vivo. In certain embodiments, the organ is a substitute organ. As used herein, a substitute organ can be an entire or partial organ, or tissue, or material that is engineered to perform one or more functions of an organ (but not necessarily the entire function of an organ). In some embodiments, a substitute organ may be produced to replace or supplement one or more functions of any organ or tissue, including but not limited to an adrenal gland, appendix, artery, brain tissue, bladder, bone, bronchus, cartilage, cornea, diaphragm, esophagus, eye, or more endocrine glands, fallopian tube, gallbladder, heart, hypothalamus, intestine, kidney, larynx, ligament, liver, mammary gland muscle, nerve, pancreas, pharynx, pineal body, lymph node, lung, spleen, stomach, ovary, parathyroid gland, pituitary gland, prostate, testicle, thymus, trachea, ureter, uterus, urethra, urinary bladder, vein, other organ, or any combination thereof.

It should be appreciated that a substitute organ may refer to an organ that is engineered ex vivo, in a bioreactor that is not connected to a body. However, a substitute organ also may refer to an organ that is grown in association with a body, for example in a bioreactor that is implanted in a subject.

Methods and devices of the invention may be used to grow, store, and/or transport any suitable substitute organ or tissue regardless of its source. In some embodiments, a substitute organ or tissue may be initiated by populating a scaffold with appropriate cells. In certain embodiments, a substitute organ or tissue may result from the growth and development of an initial set of cells without the aid of an external scaffold. In certain embodiments, a substitute organ or tissue may be provided by starting with an existing organ or tissue or portion thereof (e.g., from a donor) and incubating it in a reactor to promote further growth and/or development. However, it should be appreciated that certain embodiments described herein also may be used to promote the functional health and viability of a fully grown organ that was obtained from a donor.

In some embodiments, a substitute organ may contain one or more cell types that are being regenerated on a scaffold to form the organ substitute. The scaffold may contain biological and/or artificial material (e.g., biological and/or artificial polymers). In some embodiments, the scaffold may consist entirely of biological material (e.g., one or more biological polymers). In some embodiments, the scaffold may consist entirely of artificial material (e.g., one or more synthetic polymers). In some embodiments, a scaffold may include a mixture of one or more biological materials and/or one or more artificial materials. In some embodiments, the materials are shaped (e.g., on a template, in a mold, or using any other suitable shaping technique, or any combination thereof) to have a suitable conformation (e.g., a three-dimensional conformation) and size (e.g., volume of cells, diameter and/or length of blood vessels, airways, and/or other ducts, etc.). It should be appreciated that the conformation and/or size of a substitute organ may depend on the intended application (e.g., whether the substitute organ is intended to replace an existing organ that will be removed or whether it is intended to supplement one or more functions of a deteriorating or failing or partially failing organ that remains in the patient).

Scaffolds:

As noted herein, cells, tissues and/or organs may be grown on a scaffold that is positioned within a chamber of a bioreactor as described herein. In growing tissues and/or organs of the body, different types of cells can be arranged proximate a scaffold in sophisticated organizations or architectures that are responsible for the complex functions of the tissue or organ. Thus, architectures having dimensions and arrangements closely related to the natural conditions of the tissue or organ can be formed. The design of the scaffold and the arrangement of cells within the scaffold can allow functional interplay between relevant cells, e.g., between cells cultured on the scaffold and those of the host environment. These factors may also enable appropriate host responses, e.g., lack of blood clotting, resistance to bacterial colonization, and normal healing, when implanted into a mammalian system.

A variety of different scaffolds can be used for seeding, growing, supporting, or maintaining cells, tissues, and organs as described herein. A scaffold can have any suitable shape and may depend on the particular tissue and/or organ to be grown. For example, the scaffold may be substantially tubular, substantially cylindrical, substantially spherical, substantially planar, substantially ellipsoidal, disk-like, sheet-like, or irregularly shaped. The scaffold can also have branching structures, e.g., to mimic arteries, veins, or other vessels. In certain embodiments, at least a portion of the scaffold is hollow.

Scaffolds may be formed of natural and/or artificial materials. Materials used to form scaffolds may be biocompatible, and can include synthetic or natural polymers, inorganic materials (e.g., ceramics, glass, hydroxyapatite and calcium carbonate), composites of inorganic materials with polymers, and gels. All or a portion of a scaffold may be formed in a material that is non-biodegradable or biodegradable (e.g., via hydrolysis or enzymatic cleavage). In some embodiments, biodegradable polyesters such as polylactide, polyglycolide, and other alpha-hydroxy acids can be used to form scaffold. By varying the monomer ratios, for example, in lactide/glycolide copolymers, physical properties and degradation times of the scaffold can be varied. For instance, poly-L-lactic acid (PLLA) and poly-glycolic acid (PGA) exhibit a high degree of crystallinity and degrade relatively slowly, while copolymers of PLLA and PGA, PLGAs, are amorphous and rapidly degraded. A portion of a scaffold that is biodegradable may, in some embodiments, degrade during the growth of cells, tissues and/or organs in the bioreactor. In other embodiments, degradation may take place after implanting the tissue or organ in a recipient.

A scaffold may, in some cases, be formed of a biological material, such as a tissue construct. In certain embodiments, at least a portion of the tissue construct is acellular. In certain embodiments, the at least partially acellular tissue construct comprises tissue that has been decellularized. In the description herein concerning the use of appropriate materials to fabricate scaffolds, those of ordinary skill in the art can select materials, techniques, etc. based upon general knowledge of the art and available reference materials concerning certain techniques for fabrication, in combination with the description herein. In some cases, combinations of natural and artificial materials can be used.

Appropriate systems and techniques for fabricating scaffolds include, but are not limited to, molding, three-dimensional printing (e.g., three-dimensional layering), multi-photon lithography, stereolithography (SLA), selective laser sintering (SLS) or laser ablation, ballistic particle manufacturing (BPM), and fusion deposition modeling (FDM). Other fabrication techniques are also possible.

Scaffolds may be porous or substantially nonporous. In some instances, the wall of a scaffold includes pores having a cross-sectional dimension of less than or equal to 1 mm, less than or equal to 100 microns, less than or equal to 50 microns, less than or equal to 40 microns, less than or equal to 30 microns, less than or equal to 10 microns, less than or equal to 5 microns, less than or equal to 1 micron, or less than or equal to 100 nm. A variety of techniques can be used for introducing porosity into a scaffold. For instance, porosity can be induced by methods such as solution casting, emulsion casting, polymer blending, and phase transition induced porosity.

Scaffolds can have various dimensions which may depend on the particular use of the scaffold. A scaffold may have an average thickness of, for example, between 1 micron and 1 mm, between 10 microns and 0.5 mm, between 1 mm and 5 cm, between 1 mm and 1 cm, between 1 cm and 10 cm, or between 1 cm and 5 cm. Other thicknesses are also possible. The largest cross-sectional dimension of the scaffold can also vary from, for example, between 1 micron and 1 mm, between 10 microns and 0.5 mm, between 1 mm and 5 cm, between 1 mm and 1 cm, between 1 cm and 10 cm, between 1 cm and 5 cm, between 1 cm and 20 cm, or between 10 cm and 20 cm. A length of the scaffold can also vary from, for example, between 1 mm and 5 cm, between 1 cm and 10 cm, between 1 cm and 5 cm, between 1 cm and 20 cm, or between 10 cm and 20 cm. Other lengths are also possible. A scaffold may also have an aspect ratio (length to average cross sectional dimension) of at least 2:1, 3:1, 5:1, or 10:1 or more. It also should be appreciated that the size and thickness of a scaffold may vary over its shape (e.g., length, width, etc.). In some embodiments, a scaffold may include a series of zones of different thicknesses (e.g., forming a pattern of different thicknesses that may provide different structural properties).

Optionally, surface properties of a scaffold can be modified by various techniques. For example, in some cases, surfaces of a scaffold can be modified by coating and/or printing an additive proximate the structure. Surfaces may be modified with additives such as proteins and/or other suitable surface-modifying substances. For example, collagen, fibronectin, an RGD peptide, and/or other extracellular matrix (ECM) proteins or growth factors can be coated onto the scaffold, e.g., to elicit an appropriate biological response from cells, including cell attachment, migration, proliferation, differentiation, and gene expression. Cells can then be seeded onto surfaces of the scaffold. In one embodiment, cell adhesion proteins can be incorporated into certain portions of a scaffold to facilitate ingrowth of blood vessels. In another embodiment, growth factors can be incorporated into the scaffold to induce optimal cell growth conditions that trigger healthy tissue formation within certain regions of the scaffold. In other cases, additives can be incorporated into the material used to form the scaffold (e.g., embedded in the scaffold during fabrication).

In some cases, it may be desirable to modify all or portions of a scaffold with a material that inhibits cell adhesion, such as a surfactant (e.g., polyethylene glycol and polypropylene oxide-polyethylene oxide block copolymers). For instance, areas of a scaffold where it is not desirable for cellular growth can be coated with such materials, e.g., to prevent excessive soft connective tissue ingrowth into the structure from the surrounding tissue. In some cases, modification of surface properties of the scaffold can be used to position cells at specific sites on or within the scaffold. In some embodiments, a combination of cell-adhering and cell-inhibiting substances can be incorporated into various portions of a scaffold to simultaneously facilitate and inhibit cell growth, respectively.

In some embodiments, a scaffold can be coated with a porous material (e.g., a polymer such as a gel), e.g., prior to or during the seeding of cells. A porous polymer coating a scaffold can be used for a variety of purposes. For example, a porous polymer may be used to form pores on a scaffold that is otherwise non-porous. The porous polymer may allow, for example, sustained release of an active agent from the scaffold, e.g., to facilitate cell growth and/or cell adhesion as a function of time.

As described herein, cells may be seeded on various portions of a scaffold either before or after the scaffold is positioned in a bioreactor. In certain embodiments, cells may be seeded on at least one surface or region of a scaffold (e.g., a decellularized tissue construct) such that the cells are contained within at least one structural region of a bioreactor defined by a scaffold. In certain embodiments, cells are seeded on two or more regions or surfaces of a scaffold. In certain such embodiments, the cells on the first region or surface are of the same type as the cells on the second region or surface and in other embodiments they are of different types. In certain embodiments, at least one of the cell types on at least one of the first and second region or surface is of a type normally associated with the type of tissue comprising a decellularized tissue construct in vivo.

It should be appreciated that the cell types used to seed a bioreactor of the invention should be selected based on the type of tissue or organ structure that is being grown. In some embodiments, the cells may be epithelial, endothelial, mesothelial, connective tissue cells, fibroblasts, etc., or any combination thereof. In some embodiments, cells may be stem cells, or puripotent or totipotent cells. In some embodiments, different cells may be used to seed different portions of a scaffold. In some embodiments, one or more growth factors may be provided to promote appropriate growth and/or differentiation of the cells.

Decellularized Tissue:

In some embodiments a scaffold may be derived from an existing tissue or organ. For example, a tissue or organ may be decellularized to reveal a scaffold that can then be recellularized (e.g., with one or more patient-specific or patient-compatible cells lines) to form an organ substitute that can be implanted into a patient. A decellularized scaffold may be based on any suitable organ or tissue from any suitable organism. After decellularization, the remaining scaffold provides a structure that can be used to form an organ that has the same overall size and architecture as the original organ. However, it should be appreciated that a variety of different functions may be provided depending on the cells that are used for recellularization. Accordingly, in some embodiments, an organ scaffold may be recellularized with the same cells types that were present in the original organ to restore the same set of functions as the original organ. However, in other embodiments, only a subset of the cells may be used to generate a substitute organ that only has a subset of the original organ functions. In yet further embodiments, alternative or additional cells types may be used for recellularization, thereby providing an organ substitute that has alternative or additional functional properties. Accordingly, the original architecture may be used as a support for a general organ function by, for example, providing suitable vascularization and structural support for the cells that are used to repopulate the organ or tissue structure. In some embodiments, an organ or tissue used for decellularization may be derived from the same species (e.g., from another human, for example a human cadaver). However, in some embodiments, an organ or tissue used for decellularization may be derived from a different species. It should be appreciated that the selection of the species may be based on one or more factors including: the size of the structure that is required, the degree of vascularization that is required, the likelihood of undesirable immune response (e.g., rejection) against the scaffold (even though a decellularized scaffold is less immunogenic due to the removal of cellular antigens, there is a potential for an immune response due either to the presence of a small residue of cellular antigens, and/or an immune response against the scaffold material itself). In some embodiments, a scaffold may be obtained from any suitable mammal (including a pig, goat, sheep, etc.).

It should be appreciated that a decellularized organ or tissue may be used directly as a scaffold for recellularization. However, in some embodiments, the decellularized material may be further manipulated to alter its shape and/or size, and/or to add features (e.g., structures such as tabs, additional material, shapes that are easier to suture, etc.) that may be useful to i) help support (e.g., physically support) the substitute organ during recellularization and growth in a bioreactor, ii) to assist with removing the substitute organ from the bioreactor, iii) to assist with implanting the substitute organ into the recipient, iv) to assist with providing support for and/or monitoring the substitute organ after implantation into a recipient. For example, in some embodiments, a scaffold may be shaped or modified to include extensions (e.g., tubular extensions) for support growth of longer vessels or other tubular structures, longer or larger sections of connective tissue, or other additional tissue relative to that found on a natural organ. For example, shapes for vessels or other structures that are about 10%, about 25%, about 50%, about 75%, about 100%, or about 2, 3, 4, or 5 fold longer (or more, or any intermediate value or any range between any of these values) than a typical length of the vessel or structure that protrudes from the natural organ (e.g., than was present on the scaffold resulting from organ decellularization).

In some embodiments, adipose tissue may be decellularized to provide a scaffold that can be recellularized with one or more cell lines to generate a substitute organ that has at least one property of a different organ or tissue (e.g., a liver, kidney, heart, lung, pancreatic, or other organ function).

In some embodiments, an organ or tissue may be decellurized in an expanded position. Rather than simply perfusing an organ being decellularized, a positive or negative pressure (static or cycling) may be applied to the tissue or organ being decelluarized. For example, for a lung, a negative or positive pressure may be applied to the organ so that the scaffold can expand and thus expose the scaffold to decellularizing material (e.g., detergent) in a stretched out position. For a solid organ like a kidney, a positive or negative pressure could be created to expand the organ and its tissue to expose the organ to decellularizing material (e.g., a detergent solution, for example containing SDS; an enzymatic solution, for example containing an RNase, a DNase; with or without TritonX-100; with or without EDTA or sodium-deoxycholate; or any other suitable material) in the expanded state.

In some embodiments, the status of an organ (e.g., an artificial organ) may be evaluated in an expanded or pressurized position. This may involve a pulsatile or continuous pressure (e.g., positive or negative pressure, or a cycle of one to the other).

According to aspects of the invention, if decellularization is performed using just a perfusing flow of material, the scaffold tissue may not be sufficiently expanded and exposed to the material (e.g., detergents, enzymatic preparations, or other solutions as described herein). By expanding (e.g., using a fluid or gas to pressurize internal cavities of an organ or by stretching (e.g., pulling, twisting, etc., or any combination thereof) two or more portions of the organ or tissue relative to each other, a more uniform exposure to decellularizing material may be obtained. This is advantageous for at least two reasons: i) certain tissue regions that are generally not very accessible (e.g., due to folds or other structures) can be exposed and more completely decellularized by stretching, and/or ii) over-exposure of regions that are readily accessible is reduced or avoided, because the entire organ or tissue does not need to be exposed to decellularizing material for as much time if the accessibility of “hidden” regions is increased by expanding or stretching.

It should be appreciated that the forces used to expand or stretch an organ during decellularization may be natural physiological forces or pressure (e.g., blood pressure, air pressure in lungs, forces exerted by a muscle such as a heart or other muscle, etc., or any combination thereof). It should be appreciated that natural pressure and forces are higher than those exerted by a simple perfusion with or bathing of an organ in decellularizing material. In some embodiments, a pressure or force that is either higher than a natural force or pressure may be used (e.g., about 10%, about 25%, about 50%, about 75%, about 100%, or 2, 3, 4, 5, fold higher, or higher than any of these values, or any intermediate level or range between these values) may be used provided it does not destroy the scaffold of the tissue or organ being decellularized. In some embodiments, a force or pressure that is higher than a low pressure perfusion or a bathing solution may be used even if it is slightly lower than a natural force or pressure (e.g., about 10%, about 25%, about 50%, about 75%, about 100%, or 2, 3, 4, 5, fold lower, or lower than any of these values, or any intermediate level or range between these values).

It also should be appreciated that other decellularization techniques (e.g., using mechanical or physical forces or energy, with or without other chemical compositions) may be used in combination with the expanded tissue as described herein.

It should be appreciated that positive or negative pressure may be applied using any suitable technique, including stretching with mechanical or hydraulic pressure differentials, air pressure or liquid pressure pulses, or any other suitable pressure application, or any combination thereof. It should be appreciated that different pressures may be used for different organs. In some embodiments, the applied pressure may be physiological. However, physiological pressures are not required.

Additives for Reconstituting Scaffolds and Organs:

In certain embodiments, additives can be added to a structure used or formed in a bioreactor, such as a tissue, an organ, or a scaffold. Additives may, for instance, increase a physical (e.g., strength) and/or chemical (e.g., hydrophilicity) property of the material, which can be advantageous during growth of the tissue or organ, or during or after being implanted into a patient.

Additives can be dispersed throughout the material of a structure (e.g., a scaffold), and/or can be incorporated within certain region(s) of a structure, for example by coating the scaffold or at least a portion of a tissue or organ through a gel or other layer. Additives can also be incorporated into and/or onto a structure by adsorption or by chemically reacting the additive onto a surface. Non-limiting examples of additives include bioactive agents (e.g., therapeutic agents, proteins and peptides, nucleic acids, polysaccharides, nucleic acids, and lipids, including anti-inflammatory compounds, antimicrobial compounds, anti-cancer compounds, antivirals, hormones, antioxidants, channel blockers, and vaccines), surfactants, imaging agents, and particles. If desired, additives may be processed into particles using spray drying, atomization, grinding, or other standard techniques. In some cases, additives can be formed into emulsifications, micro- or nano-particles, liposomes, or other particles that can be incorporated into the material of the structure.

In some embodiments, one or more additives may be provided to the scaffold or support structure prior to cellularization. In some embodiments, one or more additives may be provided in a reservoir (or a plurality of reservoirs) and released (e.g., using a pump, syringe, any suitable hydrostatic or chemical or osmotic pressure, or any other technique that can be controlled and activated) at a particular time or in response to a particular cue as described herein. It should be appreciated that the reservoir(s) may be located (e.g., deposited) within the chamber or in fluid connection with the chamber. A reservoir may be constructed of any suitable material as described herein. Release of material from a reservoir may be controlled using any suitable technique (e.g., via an electrical connection, a wireless connection, a mechanical control, etc., or any combination thereof). The reservoir may have a valve that can be opened to release the contents. In some embodiments, the reservoir may be removable from the chamber (e.g., disconnected after use) in such a manner that the sterility of the chamber is preserved (e.g., the reservoir may be disconnected after a valve, cap, plug, or other sealing feature is deployed to protect the contents of the chamber from external contamination).

Bioreactors:

Bioreactor devices or components described herein may be configured for monitoring and/or modulating the growth conditions of a substitute organ or tissue. A device for growing an organ may include a chamber. The chamber may have any suitable size and/or shape. A chamber may include one or more sealable openings that can be used to introduce a scaffold and/or cells for growth and/or for other procedures or manipulations. In some embodiments, the chamber is configured for monitoring and/or modulating the growth conditions within the chamber. In some embodiments, the chamber is configured for directly monitoring the conditions of the cells or substitute tissue or organ within the chamber, or for monitoring the conditions of the chamber itself, or for a combination thereof.

In some embodiments, the chamber of a bioreactor may be attached to a system for maintaining and controlling growth conditions. Accordingly, the chamber may include one or more inlet and/or outlet ports for fluid connection with a system that may contain one or more reservoirs, pumps, controllers, etc., or any combination thereof. In some embodiments, a chamber may include one or more electrical and/or fiber-optic ports or conduits. In some embodiments, a chamber may be attached to a fixed support. However, in some embodiments, a chamber may be attached to a support via a mechanism that allows for motion in one or more directions. The chamber may be motion-driven (e.g., to provide a rotating, shaking, and/or other motion). In some embodiments, a chamber may be connected to one or more axes (e.g., 1, 2, 3, or more axes) via connectors that support movement in different directions (e.g., around one or more different axes), shaking, around one axis or two axes or three axes or more). A chamber may have any suitable size. In some embodiments, the size of a chamber may be adjustable. For example it may contain a portion that can be shortened or lengthened, it may contain a portion that is manufactured from an expandable material (e.g., rubber or other natural or synthetic material that is elastic and can expand) and/or configuration (e.g., with folds or other structures in the walls that allow for expansion or contraction), or any combination thereof. In some embodiments, the size of one or more support structures within a chamber (e.g., beams, bars, hooks, axes, etc.) may be adjustable (e.g., via a telescoping or other suitable mechanism). In some embodiments, a support structure size may be fixed, but the location of one or more attachments (e.g., for attaching a scaffold, a vascular connection, a tracheal connector, etc.) may be adjustable on the support structure (e.g., along the length of an axis, beam, hook, etc.). Accordingly, the chamber itself may be adjustable and/or the organ support components within the chamber may be adjustable. This allows the reactor to be adjustable for different organs and/or to accommodate size changes during growth and/or development.

The bioreactor and an associated control system may be constructed and arranged to provide one or more different culture conditions and/or operating parameters in one or more chambers. Such differentially provided/controlled parameters/conditions may include, but are not limited to culture media type, nutrient composition and concentration, dissolved oxygen concentration, dissolved carbon dioxide concentration, cell concentration, degree or existence of cell adherence to a substrate, temperature, media movement/fluid shear stress to which cells are exposed, pH, osmolality, etc. Such parameters can be measured over time to monitor viability and/or growth.

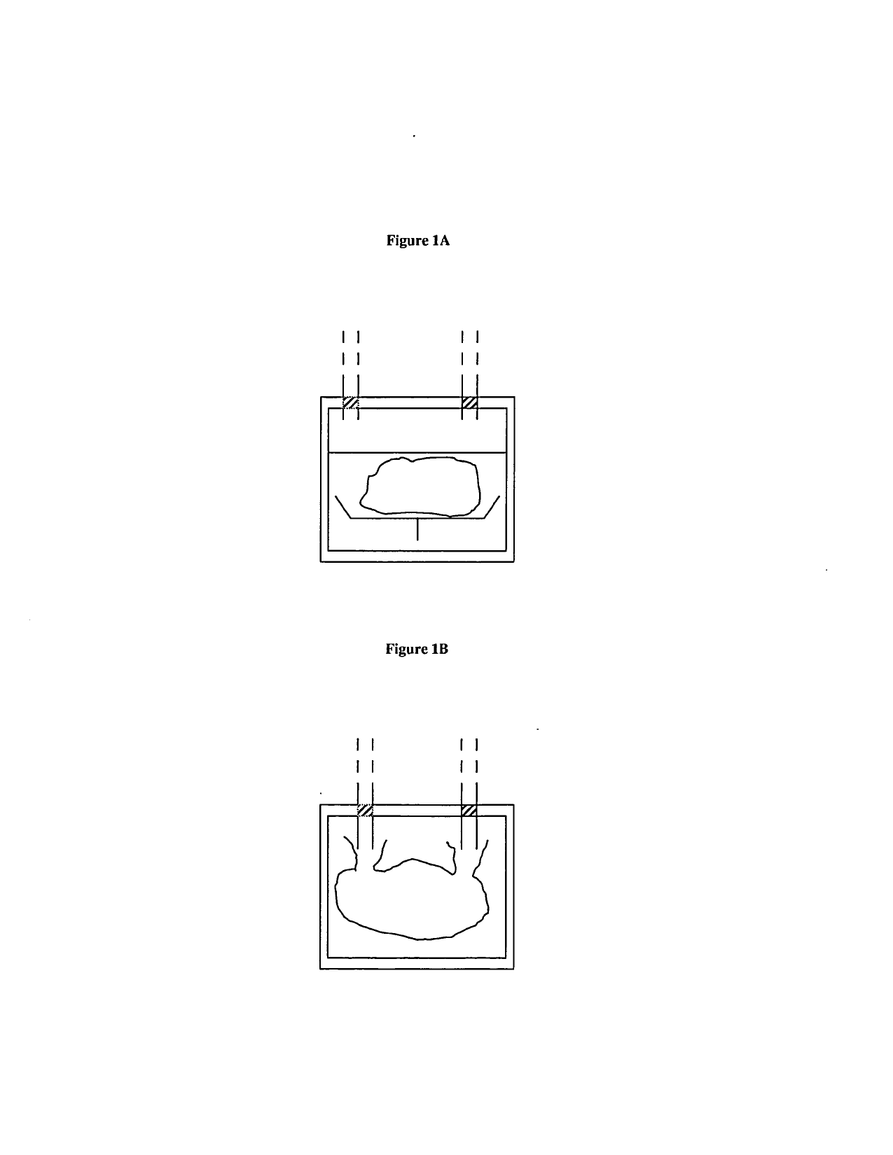

Non-limiting examples of a bioreactor of the invention are provided in FIGS. 1-3. FIGS. 1-3 illustrate non-limiting examples of a bioreactor showing individual components. It should be appreciated that in some embodiments, aspects of the invention relate to a complete bioreactor (e.g., as illustrated in the Figures and Examples) and related systems (e.g., associated controllers, databases, computers, pumps, reservoirs, supports, each of which may be physically and functionally connected to a reactor chamber). However, in some embodiments, the invention provides one or more of the component parts, or kits including such component parts. For example, embodiments of the invention may be a chamber, a support structure (e.g., a cellular support structure such as one having an axis and capable of rotating along the axis), a support structure that is hollow, a support structure that has a particular configuration of inlet(s) and outlet(s), a support structure that can be isolated from the chamber, and any one or more of the component parts (e.g., as illustrated by the non-limiting examples of component parts described and shown in the Figures and Examples), and kits including such component parts.

FIGS. 1A and 1B show side views of a schematic diagram of a bioreactor according to two sets of embodiments. The bioreactor includes a vessel wall 10, through which inlet conduit 12 and outlet conduit 14 provide a fluid communication. In FIG. 1A, the chamber defined by the vessel wall includes an organ support structure 16, on which a substitute organ 18 is shown. In this embodiment, the organ is shown completely submerged in fluid. However, in other embodiments, an organ may be partially submerged in fluid. The inlets and outlets may be connected to a system, for example, to provide for gas exchange within the chamber. It should be appreciated that one or more additional inlets and/or outlets may be included in some embodiments. An inlet or outlet may be located at different positions within the vessel wall. It should be appreciated that a vessel may have any suitable shape or size. The vessel illustrated in FIGS. 1A and 1B is shown as a closed vessel. In some embodiments, a vessel may include one or more openings for access. In some embodiments, it may include a lid on the top or a side access. FIG. 1B shows an embodiment wherein the substitute organ 18 is connected to, and supported by, inlet conduit 12 and outlet conduit 14. The ends of the inlet and outlet that are connected to the substitute organ are shown as flared. However, one or more of them may be straight, tapered, ruffled, ribbed, or have any other suitable shape. In the embodiment illustrated in FIG. 1B, the organ is shown suspended in a gas (e.g., not in a liquid medium). In some embodiments, the gas may be air. In some embodiments, it may be humidified to support viability of the organ. In some embodiments, the weight of the organ may be determined using a sensor (e.g., a scale) connected to support 16, or one or both of inlet conduit 12 and outlet conduit 14.

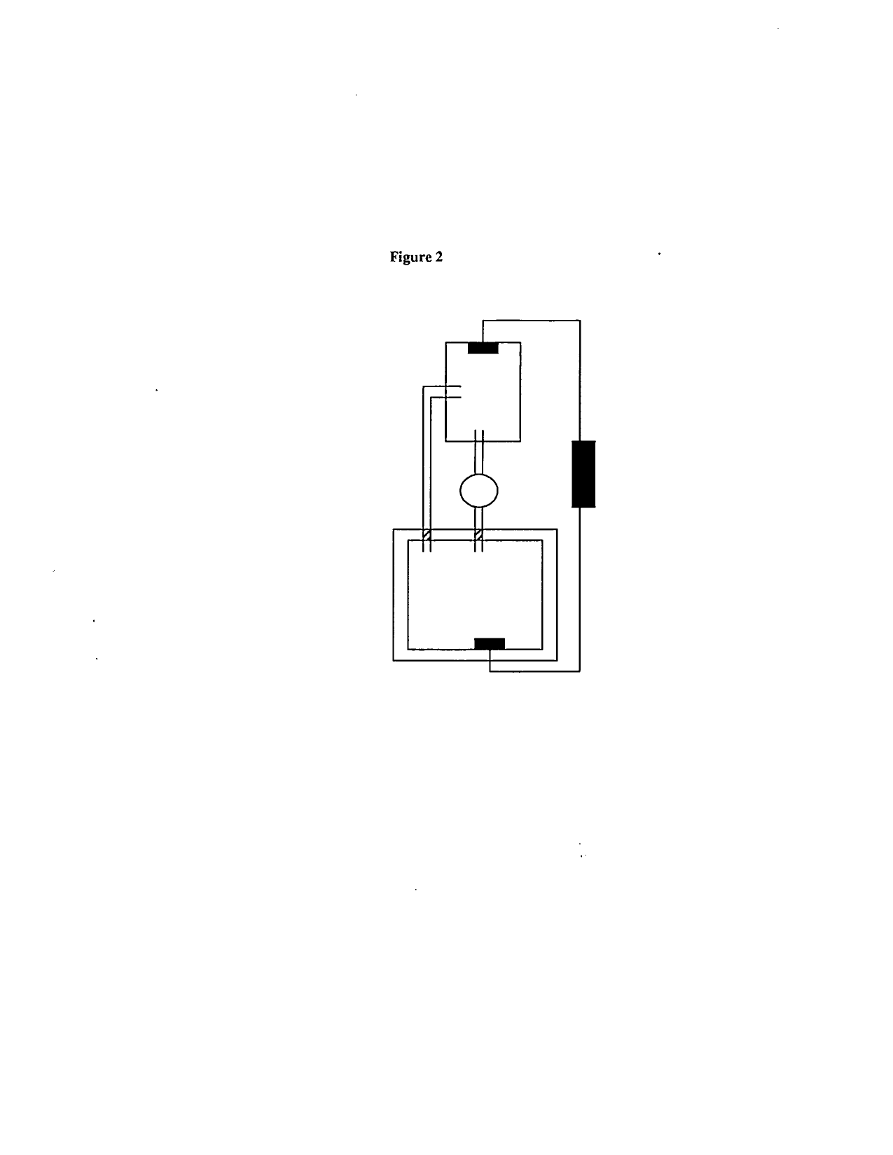

FIG. 2 illustrates a non-limiting embodiment of a system wherein outlet conduit 14 is connected via a pump 20 to a reservoir 22, which in turn is connected to inlet conduit 12. Accordingly, a closed circuit linking the chamber to the reservoir is provided. Conditions in the chamber and/or reservoir may be monitored using sensors 22. Pump 20 may be controlled by controller 24 that receives input(s) from sensors 22. As shown, controller 24 is connected via wires 26 to sensors 22. However, the controller and the sensors and/or pump may communicate via wireless, infrared, or other remote techniques. It should be appreciated that other system configurations may be provided as described herein. It should be appreciated that shapes and sizes of the conduits, chambers, sensors and other components are illustrative and other shapes, sizes, and/or components may be used as aspects of the invention are not limited in this respect.

In some embodiments, a system of the invention may include one or more mixers (e.g., static or active mixers) or other structures or devices for promoting uniform distribution of gases, nutrients, and/or other molecules within a medium. In some embodiments, a mixer may simply provide mechanical stirring. However, in some embodiments, fluid flow over the walls of one or more components may be used to mix the medium. In some embodiments, a mixer may be present in the growth chamber, in the reservoir, in a conduit, or in any combination thereof.

In general, as used herein, a component of an inventive system that is “operatively associated with” or “operatively connected to” one or more other components indicates that such components are directly connected to each other, in direct physical contact with each other without being connected or attached to each other, or are not directly connected to each other or in contact with each other, but are mechanically, electrically (including via electromagnetic signals transmitted through space), or fluidically interconnected so as to cause or enable the components so associated to perform their intended functionality.

In some embodiments, especially in certain embodiments involving growing a tissue and/or organ in a bioreactor, the vessel containing the scaffold, tissue construct, organ, or other entity is substantially closed, e.g., the vessel is substantially sealed from the environment outside of the vessel except, in certain embodiments, for one or more inlet, outlet and/or access ports that allow addition to and/or withdrawal of contents from the vessel. By maintaining a sterile seal, contamination caused by the component, such as from the external environment, may be reduced or avoided.

A vessel may have any suitable size for containing a liquid, scaffold, or other entity. For example, the vessel may have a volume from about 0.1 L and about 0.5 L, about 0.1 L and about 1 L, about 1 L and about 5 L, and from about 1 L and about 10 L. Larger volumes are also possible (e.g., 10-20 L, 20-30 L, 30-40 L, 40-50 L, or larger). The volumes may depend on the particular use of the bioreactor (e.g., the size of the scaffold, the particular tissue or organ being grown, etc.).

In bioreactors used for certain types of cell, tissue or organ cultivation, the cell, tissue or organ may require nutrients such as sugars, a nitrogen source (such as ammonia (NH₃) or amino acids), various salts, trace metals and oxygen to allow growth, division, and/or maintenance of such components. The amount of nutrients available to cells at any one time depends in part on the nutrient concentration in the liquid culture or in a solution perfusing one or more vessels of a substitute organ. It should be appreciated that a substitute organ that is vascularized may be perfused using a suitable solution that may mimic one or more features of blood (e.g., provides oxygen and nutrients and removes carbon dioxide and waste products). In some embodiments, the perfusate may be artificial blood. In some embodiments, the perfusate may be a blood product (e.g., blood or blood processed to retain certain components that are useful for oxygen, nutrient, waste product, and other transport). It should be appreciated that any suitable perfusate may be used depending on the functional requirements (e.g., having oxygen carrying properties, energy carrying properties, waste carrying properties, other properties, or any combination thereof). Sugars, nitrogen sources, salts, and trace metals may be soluble in a liquid and, therefore, may be readily available by replenishing the cells with fresh liquid media. In some cases, liquids can be introduced into one or more chambers of a bioreactor via one or more inlets described herein. The one or more inlets may be in fluid communication with one or more adjustable pumps, which are connected to sources of fluid containing appropriate combination of nutrients. In embodiments in which the percentage of different components changes depending on the stage of growth, the different components can be added together in real-time to form a media composition suitable for that growth stage. This can be done through a feedback system where one or more sensors measures the composition of the liquid(s) in the chamber(s), sends the values to a computer, which then determines what composition of fresh media is needed. After the media is formed, it can be delivered continuously or periodically to the appropriate chamber at a suitable flow rate and volume. Each chamber can include such a control system for maintaining a specified growth condition in the chamber.

It should be appreciated that one or more inlets and/or one or more outlets may be connected to a fluid containing zone (e.g., at the bottom of a chamber when in use); and/or to a gas containing zone (e.g., at the top of a chamber, above the fluid at the bottom of the chamber, when in use), and/or to afferent and/or efferent vessels of the growing organ. In some embodiments, a reactor may include any combination of two or three of these configurations. Accordingly, the external and/or internal environment of the organ may be separately and independently monitored and/or regulated through sensors, pumps, controller, and other components that may be independently connected to each of these three or more regions (external fluid, external gas, internal fluid—for example via a circulatory system, internal gas—for example via respiratory pathways, or others). Note that in some embodiments, a liquid solution may be used to perfuse airways during development (e.g., of a lung). In some embodiments, this may be replaced with a gas (and the relative pressures may be changed) at an appropriate time during development.

Like the other nutrients, even and uniform distribution of oxygen throughout the chamber(s) of a bioreactor may be essential to provide uniform cell, tissue, or organ growth. Poor distribution of oxygen can create pockets of cells deprived of oxygen, leading to slower growth, alteration of the cell metabolism or even cell death. In addition, the presence of salts plus the elevated temperature necessary to grow certain cells, tissues and organs may further reduce dissolved oxygen concentration. Since oxygen may be relatively poorly soluble or “dissolved” in water, it can be delivered to the cells by a supply of gas.

In other embodiments, oxygen and/or other gases can be introduced into a bioreactor to compensate for depletion of oxygen or other gases. As described herein, a bioreactor may include a port that is dimensioned for connection to different sources of gas, which may be independently controlled. The type of gas, number of ports, and types and configurations of ports of a bioreactor may depend, in part, on the particular processes to be carried out and cells/tissues/organs to be grown. In one embodiment, a bioreactor includes sources for different types of gases such as a dissolved oxygen control gas for controlling the amount of dissolved oxygen in the culture fluid and a pH control gas for controlling the pH of the culture fluid. For example, carbon dioxide may be used to increase solution pH and ammonia may be used to decrease solution pH. In one embodiment, a pH control gas may include a combination of carbon dioxide, ammonia, or other gases to control (e.g., increase or decrease) pH. Each type of gas may be introduced into and/or removed from the culture using different ports that can be independently operated and controlled.

Gases may be introduced continuously, periodically, or in some cases, in response to certain events, e.g., within a bioreactor system and/or within the vessel. For example, gas inlets may be connected to one or more sensors and a control system which is able to monitor the amount of gas introduced, pH, and/or the amount or concentration of a substance in the vessel, and respond by initiating, reducing, or increasing the degree of gas introduction of one or more composition(s) of gases.

As shown in the exemplary embodiment illustrated in FIG. 2, the vessel or chamber can be operatively associated with a variety of components as part of an overall bioreactor system. Accordingly, the vessel may include several fittings to facilitate connection to functional component such as filters, sensors, pumps and mixers, as well as connections to lines for providing reagents such as liquid media and gases.

It should be understood that not all of the features shown in FIGS. 1-3 need be present in all embodiments of the invention and that the illustrated elements may be otherwise positioned or configured. Also, additional elements may be present in other embodiments, such as detectors and other elements described herein. For example, a detector that can detect infrared radiation, radiation from the visible range, ultraviolet range, fluorescence, radiation from a non-visible contrast agent, or a system that can perform vibrational analysis, pressure measurements, temperature analysis, Raman analysis, electrical analysis, or combinations thereof can be integrated with a bioreactor described herein. In certain embodiments, a series of chambers and scaffolds can be positioned within a vessel. The scaffolds may be mounted on a single axis or support structures in series, or on different axes or support structures in parallel. Each chamber can have different (or the same) growth conditions, allowing multiple tissues and organs to be grown substantially simultaneously. In other cases, a single scaffold can be exposed to at least 2, 3, 4, 5, or 6 different chambers for growing the same or different cell types across different portions of the scaffold. The parameters in each of the chambers can be independently controlled as described herein to form complex tissue and organ architectures. In some embodiments, partitions or other seals may exist or be controllable to allow isolation of each chamber so that each one is individually addressable (e.g., using separate sensors, input and output conduits, stimulators, etc., or any combination thereof).

As described herein, in some embodiments, aspects of the invention relate to growing cells to form cellular tissues, organ-like structures, and/or complete organs within the bioreactor. In some embodiments, the tissue or organ-like structures are grown to form cavities surrounded by a cellular layer. In some embodiments, the tissue or organ-like structures are grown in the form of tubular structures. In some embodiments, the tubular structures may be airway structures (e.g., trachea, bronchi, bronchioles, or other airway passages), blood vessels (e.g., arteries, veins, vessels, capillaries), tubular portions of other organs (e.g., kidney, oesophagus, gut, stomach, intestine, colon, large intestine, small intestine, ducts, pancreatic duct, bile duct, gall bladder, bladder, urethra, urogenital structures, oronasal structures). It should be appreciated that tubular structures of the invention do not necessarily form perfect geometrical tubes. The shape of a tissue may be varied. In some embodiments, body cavities surrounded by a cellular layer may be created. For example, structures that mimic alveoli, heart cavities, kidney cavities, other organ or body cavities (e.g., ones that contain more or less actual tubular regions) may be grown or assembled according to aspects of the invention. It should be appreciated that the size of a tubular structure may be determined by the size of the support on which it is grown. Accordingly, the diameter and/or length may be determined by specifying the diameter and/or length of the acellular support (e.g., support matrix). Accordingly, a tubular tissue structure grown on the support may only represent a partial length of a tubular structure in a subject. For example, a length of airway or blood vessel grown in a bioreactor may be a portion of the length of the corresponding airway or blood vessel in a subject (e.g., in a human or animal).

In some embodiments, a bioreactor is used to grow a tubular structure that corresponds to a tubular structure in the body. However, in some embodiments, a tubular structure grown on a bioreactor may be used to generate a sheet of tissue (e.g., skin, membrane, sheath, connective tissue, epithelial tissue, etc., or a combination thereof). For example, after growth the tubular structure may be cut or otherwise manipulated to generate a portion of tissue that can be used as a sheet of tissue, or to form a cellular sac or bladder (e.g., by cutting, shaping, and/or suturing one or more portions of tubular structure(s) grown on a bioreactor) or other cavity surrounded by cells. Accordingly, in some embodiments, a tubular structure of the invention may be used to replace a corresponding body part (or a portion thereof) in a subject (e.g., a human or animal patient). In some embodiments, an injured or disease tubular body structure, or an injured or diseased portion of a tubular body structure is replace surgically using a tissue grown in a bioreactor according to aspects of the invention. In some embodiments, one or more tubular structures grown on a bioreactor may be used to form an artificial structure that can be used to replace a portion of an organ or tissue without specifically recreating or mimicking the actual body structure. In some embodiments, one or more tubular structures grown on a bioreactor may be used in vitro to grow additional organ or organ structures (e.g., they may be used to seed cells for further organ growth).

In some embodiments, one or more growth parameters may be similar to physiological conditions (e.g., temperature around 37° C., physiological pH, etc.)

Systems for Monitoring and Tracking Organs and Patients:

In some embodiments, aspects of the invention relate to features that are useful for high volume organ growth and transplantation, including tracking and matching of organs and patients, verification of organ/patient identity, sterility and/or the reproducibility of growth conditions. Additional aspects include options for providing patient specific devices for organ growth and/or options for providing generic organs that may be available for emergency transplantations. Each of these aspects may be implemented in a commercial process or system that supports high volume organ growth and transplantation.

In some embodiments, aspects of the invention relate to systems for monitoring and/or tracking organs and/or organ growth events. In some embodiments, tracking and matching organ and patient identity is important for managing high volume organ growth and transplantation procedures.

In some embodiments, aspects of the invention relate to methods, devices, and systems for confirming the identity of a substitute organ and/or confirming that it matches the identity of the intended recipient.

In some embodiments, the identity of a substitute organ may be determined by confirming that it is derived from the patient's own cells (e.g., it did not result from cells derived from another subject for which a substitute organ also was being generated in the same facility). In some embodiments, a DNA match test may be performed. In some embodiments, proteins or other markers may be used for identity tracking and/or matching. In some embodiments, natural markers may be used. In some embodiments, one or more artificial or synthetic markers may be used. In some embodiments, one or more artificial markers may be introduced into the cells and/or bioreactor, and, optionally the intended recipient. In some embodiments, markers may be introduced at the time of a biopsy (e.g., to remove cells from the intended recipient to use to form the substitute organ). In some embodiments, one or more separate portions of tissue may be grown inside a bioreactor along with a substitute organ in order to generate material that can be used for a match test without having to remove any of the substitute organ for testing.

It should be appreciated that useful tests could be implemented using microarrays, sequencing, PCR, RT-PCR or any other DNA detection technology. However all these techniques are relatively lengthy, expensive and often take at least several hours to run. They also involve significant sample preparation steps (e.g., lysing the cells, purifying the DNA) that may introduce errors.

Accordingly, simpler and quicker tests may be used. In some embodiments, a DNA or other marker match test is designed to be sufficiently quick and simple to be performed within a reasonable time (e.g., essentially real-time) when both the patient and organ are already in the operating room. Maintaining a “line of sight” between the patient, the substitute organ and the test minimizes the risk of error. In some embodiments, a test may involve a swab that can be swiped on the patient and swiped on the organ. In some embodiments, if the DNA or other marker is a perfect match then the swab provides a positive signal (e.g., it turns green). In some embodiments, a different signal is generated if a mismatch is detected (e.g., it turns red). In some embodiments, a test may be based on an entire genome. However, a subset of a genome (e.g., a set of specific markers or loci) may be used provided the subset provides sufficient information to discriminate between different genomic sequences and can be used to specifically match organs to recipients. In some embodiments, an assay confirms either a sequence match or a sequence mismatch, rather than determining actual nucleic acid sequences.

In some embodiments, one or more optical markers or profiles may be used to identify a substitute organ and/or match a substitute organ to an intended recipient. An optical analysis (e.g., of a substitute organ and the intended recipient) may be performed using any suitable wavelength (e.g., infrared, near-infrared, other wavelengths of radiation described herein, etc., or any combination thereof).

In some embodiments, organ and/or patient tracking may be accomplished using one or more systems that include a lock and key; a barcode; or other identifying tags to match organ and subject. In some embodiments, a tracking and/or matching procedure includes labeling a patient prior to removing cells for organ growth and providing an identity tag that can be used to match the substitute organ to the patient at a later date. In some embodiments, a tracking and/or matching procedure includes labeling cells (e.g., with a nucleic acid, a dye, or other label) at the time they are removed from a subject, and providing an identity tag that can be confirmed in the substitute organ at the time of transplant surgery. In some embodiments, a tracking and/or matching procedure includes multiple safeguards to track the organ during growth and maintain its identity and match it with the recipient of the transplant.

Accordingly, one or more device components (e.g., the chamber and/or other components of a bioreactor device) may include a component of a system for identity detection and/or matching (e.g., one or more assay components). In some embodiments, a bioreactor device may include an electronic mechanism for storing and/or communicating identity information. For example, the information in the device may be compared (e.g., electronically and/or remotely) with information associated with the patient (e.g., in a chip, on a bracelet, in the patient records, or in any other suitable form, or any combination of two or more thereof). If the information matches (e.g., is identical, complementary, or somehow indicates a match) then the implantation of the substitute organ may be performed. In the absence of a match, the implantation is not performed. It should be appreciated that a match may be used to confirm that the cellular material in the substitute organ is derived from the patient. However, in some embodiments, a matching assay or procedure may be used to confirm that a substitute organ and an intended recipient are compatible (and not necessarily identical). Compatibility may be based on one or more immunological matches (e.g., HLA matches, etc.). However, other molecules or techniques may be used to determine compatibility and/or identity, as aspects of the invention are not limited in this respect. It should be appreciated that compatibility (e.g., as opposed to identity) may be used in situations where a substitute organ is required in an emergency and there is insufficient time to grow a substitute organ from a patient's cells. Is this situation, one or more organs having different profiles (e.g., different immunological profiles) may be available and tested to determine whether they are compatible with the subject in need of a substitute organ.

In some embodiments, a device may include one or more secondary growth loci where cellular material may be maintained or grown using the same starting cell sample as was used for the substitute organ at a first primary growth locus. Cellular material at a second growth locus may be grown as a cellular mass or suspension without any organ specific support structures or components. The cellular material at a second growth locus may be used primarily to assay for identity or matching purposes without needing to disturb the substitute organ or remove a biopsy from it. It should be appreciated that an assay may be performed in situ at a second growth locus. However, in some embodiments, a sample may be removed from the second growth locus and assayed outside the reactor chamber. In some embodiments, a sample may be removed without contaminating the growth environment of the substitute organ. For example, the secondary growth locus may be isolated from the portion of the chamber where the substitute organ is being grown. In some embodiments, a device includes an access port and/or a biopsy sampler that can be used to remove cellular material from the secondary growth locus in a sterile fashion. In some embodiments, medium containing cells is removed from the second growth locus and tested for identity or matching purposes.

As discussed herein, an assay for matching compatibility and/or identity may be performed at the time and/or location of a surgery (e.g., in an operating room or in a center where the implantation procedure is being performed).

In some embodiments, the risk of an identity error can be reduced by using a procedure that involves as few manipulations as possible, particularly if the procedure is designed to minimize the number of times that cells, tissue, or an organ are transferred from a first container to a second container (e.g., from a growth reactor to a transport or storage container, or between any other containers). Each time material is transferred from one container to the next, there is a risk that a labeling error (e.g., on one of the containers) will result in an identity mismatch at the time of surgery.

In some embodiments, the risk of an identity error is managed by using a chamber that is designed to be used at two or more stages (e.g., for two or more of the following procedures: decellularization of a matrix, recellularization of a matrix, growth, storage, and/or transport). In some embodiments, the same chamber is used for the entire procedure starting with decellularization or starting with recellularization through to transport to and/or storage at the surgical location. This allows the identification (e.g., matching) of the biological material (e.g., cells, matrix, substitute tissue or organ) for the intended recipient to be performed once at the initiation of the process. The identity can then be attached to the chamber and maintained through to surgery when the substitute tissue or organ is removed from the chamber and transplanted into the recipient. Accordingly, in some embodiments a chamber is designed to be removably attached to one or more controllers, detectors, and/or other components of a system so that the chamber can be used in connection with a first growth system or device, and then transferred to a storage and/or transportation system or device and/or transferred to a surgical site without removing the substitute organ or tissue from the chamber. In some embodiments, a multistage modular chamber may be used to avoid removing an organ from the chamber prior to the time at which it is implanted into a recipient. For example, a chamber may include several regions or zones that are designed for different stages of development. As the organ develops, it may be move from a first zone to a second zone and the first region may be sealed off using a sealing mechanism (e.g., using any suitable closure device such as a valve, hatch, door, or other configuration) that is present within the chamber and can be activated at an appropriate time to seal and separate two or more different zones or regions. In some embodiments, a first part of the chamber bounding the first zone is removed (e.g., detached or disconnected) after use and after being sealed off from the second zone. It should be appreciated that the different zones may have different features that make them adapted for different applications. The different features may include one more of the following: different sizes and shapes, different configurations of attachments and supports, different configurations and numbers of inlet and outlet conduits, different types and configurations of sensors, different material in the walls, etc., or any combination thereof. For example, a growth zone may be configured to include transparent and/or flexible wall regions as described herein, whereas a storage or transport region may have thicker and insulating walls. Similarly, a growth region may have a mechanized or articulated support structure and more inlets and outlet and more sensors, or any combination thereof, than a storage or transport part of the reactor.

In some embodiments, a chamber includes a mechanism for confirming that the organ has not been removed or tampered with during growth, transportation, and/or storage. In some embodiments, the mechanism is a physical mechanism that prevents the chamber from being opened until it reaches the surgical site (e.g., using a lock that is controlled by a key or by electronic information that is provided separately). In some embodiments, the mechanism provides a signal if the chamber has been opened. The signal could be an electronic or physical signal that indicates that a chamber has been opened (e.g., an alarm, trip, interlock or other suitable component may be used to generate a visible or audible signal, for example, a light, a flag, a beep, etc., or any combination thereof) or a signal that can be recorded (e.g., an entry in a database, a code, or other information) and identified at any suitable time (e.g., prior to surgery). In some embodiments, a “seal” is affixed to the chamber in such a way that it is broken when the chamber is opened thereby providing a signal that the chamber has been opened. In some embodiments, the chamber includes a lock or seal that is broken upon opening the chamber and that does not allow the chamber to be closed again (thereby preventing the chamber from being opened and closed prior to surgery). It should be appreciated that the lock or seal may be affixed to the chamber or activated after initial identification and after cells, tissue, or an organ are added to the chamber.

In some embodiments, the identity (e.g., the intended recipient, the source of the cells, the genotype, HLA information, etc., or any combination thereof) of a substitute organ or tissue may associated with the organ as opposed to being associated with the chamber or reactor system. In some embodiments, one or more cells in the substitute organ or tissue may be labeled (e.g., with a coded tag, an electronic tag, a molecular tag, a dye, etc., or any combination thereof) to provide the information or to provide a code that can be used to obtain the relevant information from a database. However, it should be appreciated that it may be difficult or undesirable to label one or more cells in a substitute organ or tissue. Accordingly, in some embodiments, a substitute tissue or organ support structure is designed to include a region that is labeled (with the identity information or an identity code as described herein) and into which cells and tissue grow. For example, this region could be a cylinder, tube, or any other regular or irregular cavity into which the tissue may grow. The identity information or code may be attached to the wall of this cavity. In some embodiments, this region is not part of the substitute tissue or organ that will be transplanted. In some embodiments, this region could be at an exposed end of any part of the organ that can be removed prior to surgery (e.g., it could be an extension of a vessel or other structure that can be excised prior to implantation).

In some embodiments, the challenges associated with organ monitoring and/or matching may be avoided by growing an organ in situ (e.g., in a bioreactor connected to a patient, for example implanted in a patient).

Sterility:

In some embodiments, aspects of the invention relate to providing a sterile environment for the growth of a substitute organ. Systems and procedures that enhance sterility provide a significant advantage in a high volume system.

In some embodiments, methods and devices are provided to demonstrate that an organ is sterile by showing that the organ has been grown in compliance with sterile techniques. However, in some embodiments, methods and devices include a test or assay that allows for positive confirmation that a substitute organ is in fact sterile. In some embodiments, the presence of one or more contaminating cells or microorganisms (e.g., bacteria, viruses, fungi, yeast, other contaminating unicellular organisms) and/or contaminating multicellular organisms may be tested for directly. In some embodiments, a test may assay for the presence of one or more contaminating molecules indicative of the presence of a contaminating organism (e.g., protein, DNA, RNA, and/or other metabolic traces of a contaminating cell or organism).

In some embodiments, aspects of the invention relate to procedures for ensuring sterility. In some embodiments, a device may include one or more ports and/or tools that allow material to be added to and/or removed from a reactor chamber under sterile conditions. In some embodiments, a sterile closed system is provided that contains all material required for organ growth, testing, etc., or any combination thereof. In some embodiments, a closed system contains all the material required for initial growth of a substitute organ (e.g., prior to challenging the substitute organ to determine that it has one or more desired functional and/or structural properties). In some embodiments, a multistage system may be sterilized and include different zones attached to different sensors, inputs, outputs, reservoirs, manipulators, stimulators, etc., each of which can be disconnected or removed while maintaining sterility.

It should be appreciated that aspects of the invention relate to kits that contain prepackaged material (concentrate, etc., that may be sterilized) that can be added to a bioreactor; sterile/sterilizable connectors for sampling and/or adding material; filters for continuous filtering under sterile conditions; other components required for sterile growth or testing, or any combination of two or more thereof.

In some embodiments, a device may include one or more features for protecting and/or confirming the sterility of the contents. In some embodiments, a mechanism may be provided for confirming that the reactor chamber has not been opened. In some embodiments, the mechanism is a physical mechanism that prevents the chamber from being opened until it reaches the surgical site (e.g., using a lock that is controlled by a key or by electronic information that is provided separately). In some embodiments, the mechanism provides a signal if the chamber has been opened. The signal could be an electronic or physical signal that indicates that a chamber has been opened (e.g., an alarm, trip, interlock or other suitable component may be used to generate a visible or audible signal, for example, a light, a flag, a beep, etc., or any combination thereof) or a signal that can be recorded (e.g., an entry in a database, a code, or other information) and identified at any suitable time (e.g., prior to surgery). In some embodiments, a “seal” is affixed to the chamber in such a way that it is broken when the chamber is opened thereby providing a signal that the chamber has been opened. In some embodiments, the chamber includes a lock or seal that is broken upon opening the chamber and that does not allow the chamber to be closed again (thereby preventing the chamber from being opened and closed prior to surgery). It should be appreciated that one or more additional mechanisms may be provided to maintain the sterility of the reactor chamber and/or to confirm that the reactor chamber has not been opened prior to surgery.

Reproducibility:

In some embodiments, aspects of the invention relate to providing reactors that allow multiple organs to be grown in parallel under similar or varied conditions. Such reactors are useful for research and development applications. Such reactors could be useful for high volume organ growth if issues of reproducibility and reliability need to be addressed by providing options for growing backup organs under a varied conditions.

In some embodiments, a plurality (for example, 2 or more, e.g., 2, 3, 4, 5, 6, 7, 8, 9, or 10 or more) of substitute organs may be grown in a single reactor. The substitute organs may be different organs, the same organ, a plurality of copies of the same organ for a first recipients (e.g., to provide backup organs if one or more does not grow correctly), a plurality of copies of the same organ for different recipient (e.g., seeded with different patient-specific cells), or any combination thereof. In some embodiments, different substitute organs may be grown under different conditions. For example, a range of conditions (wherein, one or more different parameters may be independently varied) may be used. In some embodiments, the range of conditions are used to test and evaluate the different conditions. In some embodiments, the range is used so that an optimal substitute organ may be selected (e.g., based on functional and/or structural properties).

Accordingly, in some embodiments, a device may include a plurality (for example, 2 or more, e.g., 2, 3, 4, 5, 6, 7, 8, 9, or 10 or more) of organ support loci within a single chamber. In some embodiments, a plurality (for example, 2 or more, e.g., 2, 3, 4, 5, 6, 7, 8, 9, or 10 or more) of separate bioreactor chambers may be connected to a system (e.g., including one or more pumps, processors, controllers, etc.) that supports the function of a plurality of chambers and may provide a plurality of growth conditions in parallel (e.g., all under the same growth conditions, or all or a subset of them under different growth conditions). It should be appreciated that in some embodiments, one or more secondary sites in each reactor chamber may be seeded to provide material to test subsets of biological functions/properties on tissue grown under a range of conditions.

Generic and Specific Devices:

In some embodiments, aspects of the invention relate to generic organ bioreactors that are suitable for all organs. For example, a reactor device may include appropriate support material and input/output components that can be adapted to support any substitute organ or any size of substitute organ. In some embodiments, a device may include components (e.g., having adjustable sizes and geometry). Accordingly, a device, system, or method may be structurally and/or functionally adjustable to accommodate different shapes (e.g., for different organs); different support requirements (e.g., for different organs); different sizes (e.g., based on organ type and/or based on recipient gender, age, size, etc.); different attachment configurations and geometries (e.g., for different organs or for different recipients); different growth conditions (e.g., for different organs); different functional challenges and readouts (e.g., for different organs); or any combination thereof. In some embodiments, a computer-implementable software can be used to provide suitable growth conditions for different organ systems.

However, in some embodiments, a reactor, system, or method may be designed specifically for a particular organ of interest (e.g., a heart, a lung, a kidney, a liver, a pancreas, a blood vessel, an airway, etc.) or a subset of organs. In some embodiments, a bioreactor may include a specified set of support structures that are designed for a specific organ configuration. In some embodiments, a computer-implementable software that implements growth conditions for a specified organ may be provided.

Organs for Temporary Support:

Certain organs are more likely to fail catastrophically than others (e.g., heart and lung). Also, certain accidents may lead to a catastrophic failure of an organ. Accordingly, in some embodiments aspects of the invention relate to methods and devices for producing replacement organs available for immediate transplantation. In some embodiments, a replacement organ may be provided on a temporary basis while a new organ is regenerated.

In some embodiments, substitute organs having a reduced risk for immune rejection (e.g., based on cells that are less immunogenic) may be provided, at least on a temporary basis. In some embodiments, a plurality of different substitute organs each having different immunological properties (e.g., different HLA properties) may be provided so that an optimal one may be selected for a particular subject (e.g., one that is least likely to be rejected, particularly if the substitute organ is going to be used on a temporary basis). In some embodiments, a plurality of organs of specified sizes compatible with functional or structural requirements in different subjects (e.g., based on differences of gender, age, weight, etc.) may be provided. In some embodiments, organs with different specified geometries and sizes for transplantation (e.g., with vascular connections having different sizes and geometries) may be provided. If a range of different organs are provided, an optimal size and configuration may be selected for any given subject based on subject-specific criteria (e.g., patient shape and size at the body location of the transplantation). In some embodiments, organs may be provided with flexible attachments (e.g., long lengths of vasculature, presence and larger size of additional zones of tissue or material that could be used to attach within the patient body) that would allow transplantation into many different patient sizes and shapes even if the organ itself is not optimized for a particular patient size or shape. In some embodiments, miniorgans may be provided that can supplement one or more organ functions temporarily (e.g., for temporary repair). In some embodiments, the lengths or sizes of the additional tissue may be adapted for attachment to one or more regions in recipient. The lengths also may be useful to provide flexibility for transplantation (e.g., so that an organ can be attached regardless of the shape or physical configuration at the site of transplantation). The additional lengths of vessels or tissue (e.g., in any linear direction for a 2D or 3D tissue addition) may be on the order of a few mm to a few cm (e.g., about or precisely any of the following: 1-5 mm, 5-10 mm, 1-2 cm, 2-5 cm, 5-10 cm, 10-25 cm, 25-50 cm, or longer or shorter than any of these values or of an intermediate value).

Accordingly, aspects of the invention relate to growing one or more different organs and/or storing a plurality of different organs (e.g., a predetermined standard range of different organs having a combination of different properties and/or sizes) from which an organ for temporary support can be selected based on patient criteria. It should be appreciated that such an organ may not be a perfect match for a patient, but it may be sufficient for temporary support. In some embodiments, a patient receiving a temporary organ may also need to be treated with one or more immunosuppressive compounds to prevent or minimize rejection while a suitable substitute organ is generated (e.g., from the patient's cells).

Considerations for Organ Growth:

According to certain embodiments of the invention, tissue and organ regeneration and growth processes are associated with a natural periodicity (e.g., the variation of physical, chemical, biological and physiological parameters over time). The periodicity may vary over time in an engineered tissue or organ regeneration process with different periodicities associated with seeding, growth, and maturity. In some embodiments, aspects of the invention relate to a system with measurement, control, and/or feedback components that can be adjusted to account for this periodicity. In some embodiments, one or more of the following parameters may be detected, evaluated, and/or controlled (e.g., changed or varied) via a feedback system: temperature, oxygen, CO2, osmolarity and other dissolved gasses; flow rate, pulsation (duration, interval and intensity); single-time or repeated or regular physical movement of the organ (e.g., rotation in a gravitational field; stretching—particularly for muscle and skin; compression—particularly for bone and teeth; expansion—for example for any organ that is subject to expansion due to blood or other fluid pressure, inflation of lungs or lung tissue; beating of hearts or heart tissue or other type of exercising of the tissue/organ; “vibration” of the organ according to typical body rhythms such as a heart rate—e.g., a mother's heart rate may impact a growing embryo; breathing rate; circadian rhythms like getting up and going to sleep, activity and rest, and circadian rhythms including chemical variations involving hormones and blood sugar levels; chemical composition of the medium, e.g., nutrients, growth factors, antibiotics, antifungals, scavenging agents (for example, EDTA); changes that parallel changes in the chemical composition of the amniotic fluid changes during the term of a pregnancy (e.g., change in osmolarity); and/or electrical stimulus of the medium and or organ (e.g., to mimic one or more electrical fields in the body associated with nerve stimulation and/or heart activity). Other parameters that may be monitored and/or changed during development include spatial cues (e.g., spatial proximity of other structure or organs that may impinge on the developing organ or provide a limiting space or shape), chemical cues, cellular cues, tissues or organ cues, gravitational cues (e.g., orientation relative to gravity), toxin cues (e.g., responses to different levels of toxins). All of these cues may be added, removed, or changed during organ or tissue growth. The periodicity of change of any parameter described herein may follow a natural time-dependent change cycle (e.g., circadian or other period) and/or an experimentally identified time-dependent cycle. It should be appreciated that the changes may be automated using feedback loops including one or more sensors, controllers, pumps, etc., as described herein. In some embodiments, a user also may control the changes in one or more parameters (and/or override an automated process). Examples of natural systems that may be reproduced in a reactor, at least in part, include fetal development processes and interactions with a mother (e.g., nutritional and/or detoxification changes), where the mother provides a function until the fetus is capable of performing it independently. Accordingly, in some embodiments, a series of changes in growth conditions of a substitute tissue or organ may start with all nutrition and detoxification being provided by a bathing solution and progressively transitioning to nutrition and detoxification being performed via a perfusate (that provides nutrients and/or growth factors from a source or reservoir connected to or integrated into the device, and removes waste and/or toxins to a filtration or other unit connected to or integrated into the device). Similarly, a reactor may mimic the influence of adjacent or connected organs (e.g., the influence of a liver on the development of a lung tissue, or vice versa) during development. In some embodiments, the natural conditions may be mimicked by providing cells and/or portions of other organs and or providing functional equivalents (e.g., similar functions, including periodic changes in the function) using artificial components, controllers, and/or other means as described herein.

According to certain aspects of the invention, organs may grow better in the presence of other organs growing at the same time. For example, a liver may grow better if it is grown in conjunction with kidneys. For example, a heart may grow better if it is connected to a liver and kidneys so to as to create a more normal physiological environment. The presence of one or more other organs may help maintain the normal physical and chemical balance during growth (including some of the periodicity described herein) and may also act to process and dispose of waste products such as metabolites created by the cells. In some embodiments, an artificial placenta may be grown or produced in order to support transfer of oxygen, nutrients, etc., into the chamber and/or for retrieving samples from the chamber whilst maintaining sterility. For example, this may mimic a fetal situation, where fetal DNA, proteins and cells are all detectable in the mother's blood, but the baby remains sterile.

In some embodiments, an organ type functionality may be created within a bioreactor chamber even if other organs are not grown together. For example, dialysis (kidney functionality), metabolism (liver functionality), oxygenation (lung functionality), appropriate hormone and/or other biochemical concentrations may be provided within the chamber. One or more of these functions may be provided to avoid problems associated with toxic build up and also may simulate aspects of the natural growing environment of an organ.

It should be appreciated that any one or more organs or organ regions can be connected to a perfusate flow (e.g., via conduits, etc.) or to a fluid in the chamber, or both. Different organs or organ regions may be independently connected to different perfusate flows via different conduits and pumps etc., or may be connected to the same perfusate flow (e.g., in series or in parallel) as aspects of the invention are not limited in this respect.

In some embodiments, the cell density (e.g., cell size and/or concentration) in one or more portions of the reaction chamber may be monitored.

In some embodiments, the volume of the container and/or fluid flows may be controlled as a function of the ratio or concentration of toxins to total cell density. In some embodiments, active filtering may be used to adjust the ratios to maintain satisfactory levels of toxins.

According to aspects of the invention, a bioreactor chamber may include one or more of the following features: one or more access ports for probes, seeding, and/or fluid exchange (these may be designed to maintain sterility); optical sensing; electromagnetic radiation detection (e.g., from microwaves to X rays); imaging with cameras; sensor(s) for one or more chemicals (e.g., oxygenation, CO2, urea, etc.); sensor(s) for one or more physical (e.g., temperature, turbidity, etc.) or physiological (e.g., heart rate, breath rate, etc.) parameters. In some embodiments, optical detection may be performed through one or more transparent windows into the chamber (e.g., using UV, visible, Raman, IR, near IR, mid IR, or other optical techniques such as those described herein). Further examples of detection methods are described in more detail below. In some embodiments, a ports allows sterile access into the chamber (e.g., they are lined with a flexible, elastic, or extendable material that can protrude into the chamber but maintain a barrier between the outside and the inside of the chamber). In some embodiments, one or more detectors or probes or other mechanical devices (e.g., robot arms, etc.) may be located within the chamber walls (and sterilizable along with the chamber) and activated remotely (for example, via electrical, mechanical, wireless, or other connection).

In some embodiments, pressure sensing may be performed through the use of flexible areas of the apparatus that can be used to transmit a pressure wave (e.g., a low frequency pressure wave such as a heart rate or a high frequency wave such as a sound wave (or an ultrasound wave). In some embodiments, sound waves may be used as the detection mechanism from an optical stimulus (e.g., photo acoustic spectroscopy) from the inside to the outside of the chamber without breaching the sterility of the chamber.

In some embodiments, all the connected parts of the apparatus (chamber(s), chamber zones, reservoirs, pumps, filtration units, conduits, support structures, sensors, etc.) are sterilized prior to use (e.g., in a connected configuration so that all the components can interact as described herein without breaching the sterility of the internal space). In some embodiments, the solutions and other material that are used for growth (including nutrients, challenge material, etc.) are also sterilized and provided within the sterilized apparatus. In some embodiments, the entire apparatus, including sensors, nutrient solutions, challenge chemicals (e.g., adrenaline for heart testing, etc.) can be contained inside a sterile envelope that is only breached once, when cells are seeded in the device. In some embodiments, the entire apparatus within the sterile envelope may be disposable. In some embodiments, the interior sterile space of a reaction chamber may be defined by a flexible bag (e.g., a plastic bag, for example a disposable bag). In some embodiments, the bag may have a large cavity for actually growing an organ and several smaller bags (“pods”) coming off the main bag that may act as reservoirs for nutrients, media, challenge solutions, etc., or any combination thereof. It should be appreciated that one or more pods may be designed to fit into the inside of a syringe so that the contents of the pod could be controllably ejected into the growth chamber by using a syringe pump.

Accordingly, in some embodiments the external components of an apparatus may be reusable, whereas internal components (e.g., defined by one or more plastic bags) may be designed for single use. In some embodiments, internal components may be used to store and/or transport an organ to the operating room after growth in a bioreactor (e.g., to maintain sterility until the implant surgery).

In some embodiments, a device may include one or more integrated probes. These may be attached via a flexible or permanent mounting. However, other mountings also may be used. In some embodiments, elements of the system (control, detection, physical container, media, etc.) can verify to each other that they are both the correct parts and functioning correctly. Any access to the chamber or change to the system may be automatically recorded by the system to provide a complete audit trail and to verify process compliance.

In some embodiments, a system may include feedback and control hardware and software. In some embodiments, one or more alarms, notification systems, and/or remote monitoring stations may be provided.

In some embodiments, a device may include one or more different interfaces to connect a substitute organ (e.g., clips, ties, clamps, or other fasteners). In some embodiments, a support (e.g., a cannula) may be provided to which a substitute organ attaches during growth (e.g., the cannula may include a porous surface or other structural features or surface coatings to which cells can attach during growth).

Currently an organ is often physically suspended in a chamber by being tied to a cannula using surgical thread. This tying needs to be tight in order to support the weight of the organ. Even with small light organs such as rat hearts this can lead to necrosis and death of the tissue where the pressure is applied by the suture. Such dead or compromised tissue is undesirable for a transplanted organ. Also, reducing the size of a substitute organ by cutting off compromised tissue might result in the organ being too small to be transplanted correctly into the patient. This problem would be much worse for larger organs such as human organs (e.g., heart, liver, lung, etc.) which can weigh several pounds. The challenges can be higher also if the organ moves (e.g., a beating heart) or are moved during growth (e.g., to promote strong growth, and/or during transport). In some embodiments, damage at the site of attachment may be addressed or reduced by using suitable clamping mechanisms that can take the weight without tissue compromise (e.g., a flat band-like structure or a broad clamp); intentionally adding one or more tissue extension(s) to the organ during the growth phase to enable a better fit to the patient even if part of the tissue needs to be removed; and/or providing additional support structures to support the weight of the organ. If the source of the tissue growth matrix is a manufactured fiber (e.g., electrospun fabric, a synthetic polymer, etc.) then the extensions could be designed in from the start. If the source of the tissue growth matrix is a decellularized structure (such as an animal or human organ), then the extensions could be added in before the seeding of cells. The organ could then be clamped to these extension structures for its support, thereby not compromising the tissue that is ultimately transplanted and ensuring that there is sufficient tissue to connect to the patient. In some embodiments, one or more of these extensions may be tubular extensions attached to vessels as described herein. In some embodiments, the shape of the extensions may be tapered, flared, irregular (e.g., including on or more slits or other irregular edge shapes), depending on the configuration that is useful for the final application (e.g., transplantation).

In some embodiments, organs may be grown in a physical and chemical support medium or matrix. If large organs are too heavy to be supported by traditional means of tying the top to a physical support (e.g., suturing/tying the bronchus or aorta to a stainless steel cannula) then it may be helpful to fill the chamber with a physical support medium to take the weight of the organ. This could be as simple as water or saline, though a more complex biological and/or chemical support medium like culture media or either natural or artificial amniotic fluid or interstitial fluid may be used. A fluid could be of low viscosity like water or of high viscosity like a gel. The support medium may provide both physical support and/or chemical or biological support. In some embodiments, the support medium may be designed to mimic natural body media (e.g., amniotic fluid, interstitial fluid, etc.). In some embodiments, the support fluid can be separated from the substitute organ by a flexible membrane in order to avoid direct liquid contact with the organ. However, other support structures may be used as described herein.

Growth Environment:

In some embodiments, aspects of the invention relate to providing periodical/repeated changes in conditions that can be optimized for growth and development. In some embodiments, the periodical/repeated changes may be developed to mimic aspects of the natural growth environment of an organ. It should be appreciated that other methods of mimicking the natural growth environment of an organ may include growing a substitute organ in situ in a subject (e.g., within a bioreactor that is implanted into the subject). In some embodiments, a substitute organ may be grown in a bioreactor in combination with one or more other organs or organ-like structures. In some embodiments, the support and surrounding conditions in a bioreactor may be designed to reproduce one or more natural growth conditions and changes in the conditions during growth may be used to reproduce natural growth conditions. However, it should be appreciated that natural growth conditions may not be ideal for rapid regeneration of a tissue or organ. Accordingly, growth conditions may be optimized based on one or more criteria. In some aspects, different growth conditions may be optimal for different applications (e.g., depending on whether speed of growth, structural strength, complex functions, or other criteria are more important for the application).

In some embodiments, reactor chambers may be implantable into a body to facilitate the regrowth of an internal organ. A chamber may include one or more films or gels that could be injected or placed into the organ or near the organ to help promote regeneration. For example, a kidney shaped chamber (the chamber could be biodegradable and never need explanting or could be surgically removed at a later date) containing a fluid, gel or matrix that supports or encourages the regeneration of tissue (either by favoring regeneration or suppressing scar formation) could be used to regrow either an entire kidney or a portion thereof. Similarly, entire organs, organ portions, or tissues may be regrown at other sites (e.g., liver, pancreas, lung, etc.).

In some embodiments, an organ can be removed from a patient, decellularized, recellularized and transplanted back into the patient. This may be relatively straightforward for organs that have some redundancy (e.g., humans have two kidneys and two lungs and can survive on only part of a liver or with significant loss of muscle, bone, skin, etc., and could even survive on a heart-lung machine while the old heart and or lung was decellularized and recellularized). In some embodiments, methods and devices may be used for repairing brain lesions due to accidents, stroke, injury, etc., or any combination thereof. In some embodiments, a scaffold or matrix in the form of a organ may be implanted such that the recipient body can populate it with cells.

These “inside the body” approaches provide the distinct advantage of maintaining sterility, avoiding any mismatch in organ/patient identity, providing nutrition (and metabolite and other toxin clean up) and the natural periodicity of the body's own physics, chemistry, biology and physiology. In some embodiments, one or more chambers also may be provided outside the body or attached to the body for certain organs like fingers, toes, ears, noses, limbs, other bones like ribs or skull bones, teeth and perhaps eyes (e.g., an eye could be popped out of its socket while a patch of new tissue is grown to repair either a wound or an area of the retina degraded by macular degeneration (wet or dry)). Macular degeneration in particular may be addressed by surgically removing a disk of the retina, growing a new one outside the body and then surgically transplanting it back in or regrowing the tissue inside the body or in a chamber attached to the eye while the eye is still attached to the body.

Accordingly, aspects of the invention relate to devices having flexible sizes (e.g., to provide adaptable growth environments that allow for increases in size, periodic changes in conditions, movement, etc.). In some embodiments, a growth chamber or portion thereof may include flexible and/or elastic material (e.g., balloon or sock) that can change shape and/or expand to accommodate organ growth and/or maintain growth conditions (e.g., increase medium volume to provide stable concentrations of growth material and/or waste). In some embodiments, a device may have a flexible design (e.g., it is constructed of non-flexible material, but with a design that allows for the internal volume to be changed).

Accordingly, a chamber may have a flexible shape or size, include elastic and/or flexible material, contain one or more access ports, contain one or more observation points (e.g., made with a material that allows an optical signal to pass through), include one or more attachments (e.g., for generating or transferring motion), include one or more connectors (e.g., electrical, mechanical, optical, and/or fluid (in and/or out)), and/or include one or more sensors. In some embodiments, a fixed network of monitoring sensors (e.g., optical, chemical, mechanical, etc., or a combination thereof) may be provided and placed on or in a substitute organ, within the device walls, within the matrix/scaffold or other organ support material, or any combination thereof. In some embodiments, flexible and/or adjustable (e.g., moving and/or movable) monitoring sensors may be provided. In some embodiments, an integrated monitoring system within a device can be moved and/or targeted to sample or analyze specific areas of a substitute organ (e.g., according to a predetermined schedule or allowing user specified analyses). In some embodiments, flexible monitoring sensors that adapt to a growing organ may be provided. In some embodiments, sensors may measure and or detect pressure (e.g., pressure levels, changes or differences) flow (e.g., flow rates and direction), movement, chemicals (e.g., lactate, ammonia, glucose, O2, CO2, ions, etc.), mechanical force, vibrational properties, fluorescence, light or sound, and/or temperature within the device or organ. It should be appreciated that different sensor distributions and geometries may be used. In some embodiments, one or more sensors may be distributed along the length of an organ support material; over a matrix or scaffold, have a fixed geometry (e.g., for 360 degree monitoring of an organ, and/or for monitoring particular points of interest), and/or be movable (e.g. controllable, for example, wirelessly). In addition, or alternatively, one or more sensors within the device (e.g., in a chamber, a reservoir, a conduit, a pump, etc., or any combination thereof) may be used to detect conditions within the device, growth medium, inlet or outlets, etc., or any combination thereof. Direct measurements on the tissue or organ being grown or at one or more locations within the device may be used to develop improved growth conditions or as cues that are indicative of the progress of organ or tissue development, potential problems, and/or whether the organ or tissue is ready for storage, transport, and/or surgical implantation.

In some embodiments, a system or device is provided with a mechanism for generating movement, e.g., rocking, oscillation, or more complex movements. In some embodiments, internal mechanical components may be provided for generating movement of an organ support structure or material within a growth chamber. In some embodiments, a device (e.g., a chamber) may include on or more structures for attachment to an external source of movement. Accordingly, in some embodiments, a device or chamber may include one or more axes about which it may rotate. In some embodiments, a device or system may be dynamic and adapted to respond to sensor input to maintain a predetermined set of conditions or series of different conditions (e.g., to mimic natural growth and development conditions). In some embodiments, a bioreactor may include access ports coupled to a system with suitable control and feedback for providing a dynamic growth environment. In some embodiments, devices may be provided with hardwired perfusion pathways (e.g., to flow from one part of an organ to another, to flow between organs on a device, etc., or any combination thereof). In some embodiments, an organ support matrix may be provided with sensors (e.g., optical, pH, chemospecific, electrical, temperature, mechanical, etc., or any combination thereof). In some embodiments, an organ support matrix may be provided with stimulators (e.g., electrical, mechanical, chemical, etc., or any combination thereof). In some embodiments, an organ support matrix may be provided with specific perfusion pathways built in. In some embodiments, a mesh (either within the support material, or an additional monitoring mesh that can be placed over an organ) may be provided with nodes/knots each having one or more sensors (e.g., optical, chemical, etc., or any combination thereof). In some embodiments, the monitoring mesh may be flexible (e.g., stretchable, or expandable).

In some embodiments, movement of the bioreactor, or the chamber thereof, may be useful to mix the medium within the chamber and promote homogeneous distribution of nutrients, gases, waste products, additives, etc., or any combination thereof.

In some embodiments, aspects of the invention relate to kits containing one or more materials for use with a system or device of the invention. In some embodiments, one or more components of a kit may be disposable. For example, different materials for different phases in a growth cycle (e.g., blood, for example different hemoglobin profiles, different “amniotic fluid” compositions for different growth stages) may be provided. Different containers for different materials (prepackaged, sterile, etc.) may be provided. Different filters (e.g., based on size, chemospecificity, and/or other properties) and/or filter configurations may be provided. In some embodiments, active filters may be provided for continuous filtering as opposed to replacing the medium periodically. In some embodiments, active fluid conditioning may be used to remove waste products or other undesired molecules and/or to add nutrients, growth factors, etc., and/or any other desired molecules as they become needed (e.g., either because they are depleted due to use or because they are required for a transition to a further stage in growth or development, or for other reasons).

In some embodiments, aspects of the invention relate to protocols (e.g., computer-implementable software) suitable for varying growth conditions, and/or to mimic body growth conditions, and/or to provide different growth conditions (e.g., pressure profiles) for adhesion (e.g., seeding), growth, and/or maintenance of a substitute organ.

In some embodiments, a mechanical protocol for growth may include no pulsation, but optional movement for an initial cellular deposition, pulsation after the initial deposition, and an optional stage for testing without pulsation or movement.

In some embodiments, a vacuum system may be provided to accelerate cell adhesion to a matrix or other support material.

In some embodiments, complex patterns of pulsation and/or movement may be provided to mimic an overlay of patterns representing heart beat and respiratory rhythms, and/or optional additional movements.

In some embodiments, pumps and related controllers suited for pulsatile delivery of gas and/or fluid may be provided.

In some embodiments, systems responsive to threshold levels of analytes with feedback loops to maintain predetermined growth conditions (e.g., with pumps to adjust concentrations of material) may be provided.

In some embodiments, general filtering protocols may be provided.

In some embodiments, cells growing in a chamber (e.g., on a scaffold) may be subjected to shear stress. In some embodiments, movement of the device or the chamber, and or flow of the medium over the scaffold or the cells may generate shear and/or other physical stress on the cells seeded on the support. For example, a shear flow stress of from about 0.01 to about 500 dynes/cm², from about 0.01 to about 50 dynes/cm², from about 1 to about 200 dynes/cm², from about 1 to about 100 dynes/cm², from about 1 to about 50 dynes/cm², or from about 1 to about 25 dynes/cm² may be applied to the cells. Smaller or larger values of shear stress are also possible. In some embodiments, the use of shear stress aids or promotes cell or tissue growth and/or the formation of cellular tissue with enhanced structural properties (e.g., increased elasticity, tensile strength, etc., or a combination thereof). The use of shear stress can also help to guide cell orientation and/or alignment.

It should be appreciated that the shear stress may be varied depending on the stage of cell/tissue/organ growth. For example, a low shear stress may be appropriate during the seeding of cells to facilitate cell attachment onto a scaffold. The shear stress can then be increased to higher levels after cell seeding. The amount shear stress will also depend on factors such as the size of the tissue or organ, the particular type of tissue or organ, and the particular types of cells being seeded.

In some embodiments, the gravitational field that a developing tissue or organ is exposed to may be varied by changing the orientation of the organ or tissue relative to gravity. In some embodiments, the orientation of the organ support structure within a chamber may be varied. In some embodiments, the orientation of the chamber itself may be varied (and the orientation of the organ support structure relative to the chamber may be fixed or variable in some embodiments.

It should be appreciated that sensors that monitor one or more parameters described herein (e.g., pressure, flow, pO2, pH, CO2, lactate, glucose, electrical, ion concentration, mechanical force, torque, stretch, fluorescence, emissivity, vibrational properties and/or response to external energy, temperature, imaging, and/or other information described herein). One or more (e.g., 2, 3, 4, 5, 6, 7, 8, 9, 10, or more) parameters may be monitored and/or varied to determine and select experimental and/or natural cycles of growth conditions that can be used to optimize organ development (e.g., structurally and/or functionally), and/or to optimize the speed of organ development. A multivariate analysis can be used to select conditions (e.g., with different phases in growth conditions) that provide a desired organ quality within a suitable period of time (e.g., in a large scale and/or commercial growth context where organ throughput may be important).

Organ Support:

In some embodiments, aspects of the invention relate to device configurations for supporting substitute organs during growth. In some embodiments, a device may be configured for substitute organ growth in a fluid; growth attached to a tether (e.g., hanging); and/or growth on a weight-bearing support. In some embodiments, a device may be configured to promote movement (e.g., oscillation) to promote growth, stimulate structural resilience, prevent ischemia, or any combination thereof.

Accordingly, a device may include a combination of hooks, tethers, and/or other weight support features adapted to simulate conditions and/or attachments in a recipient. In some embodiments, devices may be provided with an adjustable support. For example a support may be adjustable to provide a platform or buoyancy during early growth and be able to switch to tension (e.g., dangling) during a later stage of growth to simulate attachment conditions in a recipient. In some embodiments, one or more attachments and/or other components (e.g., the growth chamber) may have adjustable sizes and/or shapes to accommodate organ structures of different sizes and shapes. In some embodiments, one or more attachments and/or other components (e.g., the growth chamber) may be adjustable to accommodate changes in the size and/or shape of the substitute organ during growth. In some embodiments, a device may include one or more mechanical, motorized, and/or magnetized components to move an organ during growth. In some embodiments, a device may include an adjustable (e.g., inflatable, deflatable, extendable, retractable, etc.) support mechanism (e.g., a balloon-like structure) to provide adjustable support levels during growth and development of a substitute organ.

The following support structures may be used in addition to one or more platforms or beams that support different parts of the substitute organ. The following structures may be used to relieve one or more pressures on the substitute organ, for example, on the attachment of the functional (e.g., umbilical) components of the substitute organ to the reactor system.

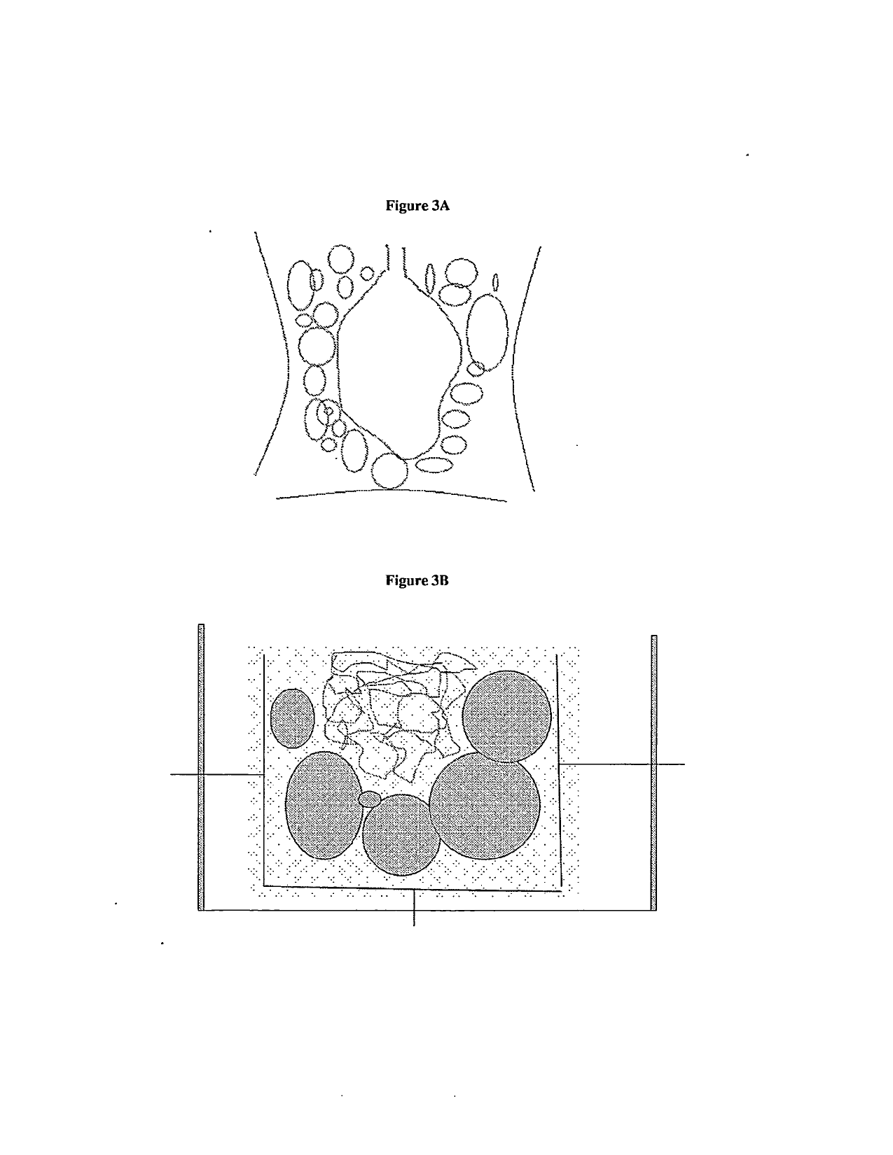

In some embodiments, a substitute organ may be supported by one or more support structures that can be placed in a chamber of a bioreactor. In some embodiments, a plurality of support structures may be included to form a bed of support structures that can support the substitute organ. An individual support structure may be a solid structure, a deformable structure, or include solid and deformable portions. A support structure may have any suitable shape for supporting an organ. In some embodiments, it may be spherical, cubical, cylindrical, or other geometric shape, or any irregular shape, or a combination thereof. FIGS. 3A and 3B illustrate non-limiting embodiments with a plurality of spherical structures 28 that are provided to support a substitute organ 18 or a scaffold 30. In this figure, the structures are shown as balls or beads. However, it should be appreciated that a plurality of structures of different sizes and/or shapes may be used. It also should be appreciated that one or more of the support structures may be immobilized (e.g., tethered) using any suitable technique (e.g., a tether, an adhesive, a reversible attachment, etc.). However, one or more of the support structures may be loose within the bioreactor chamber.

In some embodiments, a deformable structure may be fabricated of a deformable material (e.g., a gel-like material or other deformable material). In some embodiments, a deformable structure may be a first pouch that comprises an outer membrane which contains a deformable material (e.g., a liquid or a gas) or a plurality of smaller components (e.g., beads, balls, or other shapes), or a combination thereof. The smaller components may be solid, gel-like, smaller pouches, or any combination thereof. The first pouch may have any suitable shape for supporting an organ (e.g., alone or in combination with one or more other structures). In some embodiments, it may be spherical, cubical, cylindrical, or other geometric shape, or any irregular shape, or a combination thereof. Similarly, the shapes of the smaller components within the first pouch may be spherical, cubical, cylindrical, or other geometric shape, or any irregular shape, or a combination thereof. The deformable structure may be adapted and arranged for certain detection techniques, such as infrared detection, detection in the visible range, detection of absorbance, transmission, and/or reflectance, temperature monitoring, detection of pressure, vibrational analysis, Raman analysis, fluorescence detection, electrical analysis, or other detection methods described herein.

In some embodiments, a deformable support structure may have a shape that can be changed, for example, using a controller and/or an automated procedure. In some embodiments, the shape may be changed by inflation or deflation of one or more parts of the structure (e.g., using a gas and/or liquid). In some embodiments, the shape may be changed using a mechanical mechanism or other mechanism housed within the structure. Accordingly, in some embodiments a deformable structure may be connected to a controller, pump, and/or other device via a wire, cord, tube, or any combination of two or more thereof. In some embodiments, the shape of a deformable structure may be controlled remotely, e.g., using a wireless receiver, an infrared sensor, or any other suitable remote control mechanism housed within the deformable structure.

In some embodiments, a support structure (whether deformable or not) may include one or more sensors. For example, a support structure may include a strain gauge or other sensor for detecting the pressure exerted on the structure. In some embodiments, a support structure may include one or more other sensors to detect chemical and/or biological signals within the reactor chamber. In some embodiments, the weight of the support structures is known and the weight of the organ supported by the structures may be determined by measuring the total weight of the substitute organ along with the support structures (e.g., using a balance, scale, or other sensor that is attached to support platform within the reactor chamber that supports the support structures in addition to the organ). The weight of the organ can then be determined by subtracting the known weight of the support structures.

In some embodiments, one or more support structures may be placed on the outside of an organ (e.g., within a growth medium). In some embodiments, the support structures are placed beneath and/or surrounding the substitute organ. It should be appreciated that the number and size of the support structures may be optimized to support a substitute organ of interest. A suitable combination of structures of different sizes and/or shapes may be selected to form an aggregate structure that cradles many or all parts of the organ.

In some embodiments, one or more support structures may be placed within one or more cavities of a substitute organ. For example, one or more support structures may be placed within one or more bronchial, alveolar, and/or other airway cavities of a lung. In some embodiments, one or more support structures may be placed within one or more ventricular, atrial, arterial, and/or or other vascular cavities of a heart. In some embodiments, one or more support structures may be placed within one or more vascular and/or tubular structures (e.g., ducts, etc.) of any substitute organ (e.g., liver, kidney, pancreas, etc.) or portion thereof to provide structural support during growth, regeneration, and/or maturation of the substitute organ. It should be appreciated that the one or more support structures may be removed prior to implantation. For example, the support structures may be removed when the organ is removed from the reactor chamber in preparation for surgery. In some embodiments, the support structures may be removed earlier, for example, when the substitute organ has sufficient internal structure to no longer need support.

It should be appreciated that in some embodiments the size of the structures may be reduced (e.g., by deflation or other technique) to assist in removal of the structures from the substitute organ at an appropriate time.

In some embodiments, one or more of the support structures may provide first intermittent or regular stimuli to the support organ in the bioreactor chamber. For example, a support structure may undergo changes in volume and/or pressure at regular intervals. For example, the support structure may alternately increase and decrease in volume (or provide alternating increases and decreases in pressure) to mimic one or more natural rhythms (e.g., pulsating blood pressure, regular breathing). In some embodiments, second changes in volume and/or pressure may be provided by one or more support structures to mimic the movements and/or growth of adjacent tissues or organs and the resulting pressure changes that occur during natural growth and development. These changes may be less regular, and involve fewer alternating changes, than the first changes that mimic natural rhythms such as alternating blood pressure or respiratory movements. It should be appreciated that in some embodiments, support structures provide both first and second volume and/or pressure changes.

It should be appreciated that the number and/or size and/or shape of support structures may be changed during growth of the organ. In some embodiments, additional support structures are added during growth. In some embodiments, a subset of support structures are removed during growth. In some embodiments, the size and/or shape of one or more of the support structures is changed during growth. It should be appreciated that one or more of these changes may be made to provide appropriate support and/or space for new parts of a growing and developing organ substitute. In some embodiments, a device is modular and different components attached to a chamber and/or different regions or zones of a chamber may be sealed off (e.g., in a sterile fashion) and optionally removed during development. In some embodiments, an organ or tissue may be moved from one region or zone (e.g., using a mechanized support structure, a mechanical or robot arm, or any suitable mechanism or means for moving the organ alone or along with its support structure and/or functional connections).

It should be appreciated that in use the support structures described herein may be placed with a medium, for example a growth medium, within the reactor chamber. The support structures, and/or the substitute organ, may be partially or completely submerged in the medium. Accordingly, the support structures (e.g., balls, beads, balloons, vesicles, etc.) may define a flow pathway around their surfaces that may facilitate flow and/or diffusion of reagents and/or metabolites while still providing support.

Accordingly, two or more (e.g., 3, 4, 5, etc.) different flow pathways may be provided using different zones of support structures. In some embodiments, support structures on the outside of the substitute organ may define a liquid volume, for example, that can be a flow pathway for introducing and/or removing liquid to the outside of the substitute organ. In some embodiments, support structures within one or more cavities of an organ may define a liquid volume, for example, that can be a flow pathway for introducing and/or removing liquid to the inside of the substitute organ. In some embodiments, two or more different flow pathways may be defined by different zones of support structures, internal and/or external to the organ. In some embodiments, the different pathways may be connected to separate conduits, pumps, filters, reservoirs, etc., or any combination thereof. Accordingly, the conditions in the different flow pathways may be maintained separately (for example, detoxified separately, e.g., using separate chemical or physical filtration systems, or separate dialysis systems, etc.). The operation of the separate systems may be altered as the substitute organ grows, for example, to adapt to changing nutritional and/or waste removal needs.

In some embodiments, support structures do not occupy any volume (e.g., they are not present and/or are not inflated) in the reactor chamber during the early stages of substitute organ growth while the ratio of medium to cell mass is large. The ratio of the volume of the support structures to the volume of medium may be changed as the substitute organ grows. In some embodiments, the number, location and/or volume of support structures may be adjusted (e.g., increased) during substitute organ growth. It should be appreciated that changes in the support structure size, shape, and/or location may be controlled using any appropriate techniques. Non limiting examples of useful techniques include, but are not limited to, mechanical, magnetic, pressure-based, flow-based, and/or other techniques.

In some embodiments, the volume of medium surrounding a substitute organ may be reduced as the substitute organ grows and develops (e.g., becomes vascularized), because many or all of the nutritional and metabolic functions of the substitute organ may be provided via its connection (e.g., “vascular” connection) to the reactor system. Accordingly, in some embodiments waste removal and/or detoxification can be achieved by filtering and/or treating fluid in the vascular system of the substitute organ instead of (or in addition to) filtering or treating the medium surrounding the substitute organ. However, it should be appreciated that in some embodiments, the volume of the medium may be maintained (or even increased) as the number, size, and/or relative position of the support structures change to provide optimal support during substitute organ growth. Accordingly, the medium may still be used to detoxify or remove waste (for example, via dilution due to a relatively large volume of medium, and/or by filtering and/or replacing the medium, e.g., continuously or at one or more intervals during growth).

In some embodiments, the walls of the chamber (e.g., side walls, and/or floor and/or ceiling of the chamber) may include features that allow them to exert pressure on the support structures within the chamber (e.g., to provide support and/or pressure during substitute organ growth). In some embodiments, one or more of the walls may be movable (e.g., the entire wall or a portion thereof) to exert pressure within the chamber. In some embodiments, as the volume of the chamber is reduced, one or more support structures (e.g., balls, etc.) may transfer the pressure to the organ in an organ-specific fashion (for example, if the size and configuration of the support structures is designed to be organ-specific, e.g., the aggregate configuration of the support structures effectively provides a mold with an organ-specific structure). In some embodiments, one or more walls may be flexible and pressure may be applied via the walls using an external force or mechanism

It should be appreciated that the support structures described herein may be disposable or re-usable. In use, support structures should be sterile. Accordingly, the structures may be sterilized during manufacture. Reusable support structures should be sterilizable (e.g., autoclavable, UV-resistant, and/or resistant to one or more other sterilization techniques such as chemical sterilization).

In some embodiments, a device may be adapted to be connected to a mechanical, motorized, and/or magnetic system for generating organ movement during growth. Accordingly, aspects of the invention relate to methods and systems for supporting and moving organs during growth, e.g., to prevent ischemia, to validate tethering and strength of tissue required to support the substitute organ after transplantation, to provide appropriate stimuli during growth, etc., or any combination thereof.

In some embodiments, artificial structures (or flexible reactor walls) can act as a support to assist in supporting developing organs. In some embodiments, the structures may be perfused to provide circulating oxygen or other nutritional materials to, and/or to remove waste products from, supported organ or tissue regions. Accordingly, in some embodiments, all of the organ regions (internal and external) may be in contact with appropriate perfusion solution (even if part of the organ is in contact with a support).

Accordingly, in some embodiments, a shaped support (e.g., having the shape of a part, for example a lower part, of a desired substitute organ or tissue) may help organ development. In some embodiments, the support may be porous or otherwise perfusable to provide materials and remove waste from the growing part of the organ that is in contact with the support. It should be appreciated that one or more support structures therefore may be connected to one or more inlet or outlet ports or other conduits that provide a flow (e.g., a pumped flow) of perfusate.

It should be appreciated that one or more organ support structures described herein (e.g., surfaces, beads, hooks, beams, etc.) may include a sensor (e.g., a mechanical, electrical, chemical, optical or other sensor). In some embodiments, a sensor may be a gauge (e.g., a strain gauge, a pressure gauge, or any other gauge) that can be used to detect a force (e.g., weight, torsion, pressure, tension, etc., or any combination thereof). FIG. 3C illustrates a non-limiting embodiment of an organ 18 in a reactor chamber 10 connected to two strain gauges 40. It should be appreciated that other configurations also may be used with one or more different gauges.

In some embodiments, one or more support structures may have be configured to exert (in addition to or instead of sensing) a mechanical or physical force on an organ or tissue (e.g., a pressure, a torsion, a tension, etc., or any combination thereof). Accordingly, an organ support structure may include or be connected to a motor, a pump, a clamp, an electromagnetic device, or any other source of physical or mechanical force.

Accordingly, one or more support structures may be connected to a computer, controller, database, or other electrical system (e.g., directly or wirelessly) to detect, record, and/or transmit information from a sensor or to a mechanical or physical device associated with the support structure.

Vascular Attachment:

In some embodiments, aspects of the invention relate to features for connecting a substitute organ to a bioreactor, growth/matrix, and/or cannulae (or similar connectors).

In some embodiments, the vasculature support structure (e.g., the matrix or scaffold on which the vasculature is grown) is shaped or adapted for clamping (e.g., to provide a surplus length, a tapered shape, a flared shape, etc.). In some embodiments, clamps and/or cannulae with particular shapes adapted for vascular attachment are provided. In some cases, a portion of a cannula and/or scaffold itself may have a shape and/or configuration that facilitates handling of the scaffold by a user, facilitates connection of the scaffold to a flange or other component of the bioreactor, and/or facilitates positioning or connection of the scaffold to the body of a recipient. For instance, one or more portions (e.g., ends) of the scaffold may include a handle, rod, ring, flared or tapered ends, combinations thereof, or other suitable shape and/or configuration. A tubular structure may, for example, have flared or tapered ends which can facilitated insertion and/or attachment of the structure to a component of a bioreactor (e.g., a cannula), as well as to the body of a recipient. Such a component that facilitates handling and/or connection of the scaffold may be formed of the same material or a different material as the portion of the scaffold used for growing a tissue or organ. In some cases, such a component comprises a cell resistant material so that fewer cells grow on these portions. In some instances, such components are biodegradable or resorbable after being implanted into a recipient.

In some embodiments, a device may include one or more clamps (e.g., soft clamps) and/or cannulae adapted for tethering an organ or an organ vasculature. In some embodiments, cannulae adapted for tethering an organ or an organ vasculature may be provided, e.g., with ridges, or with a porous surface (and/or a surface coating) for attachment of scaffold and/or for supporting growth of vascular tissue. In some embodiments, scaffolds and/or matrices for growing organs may be adapted for clamping or attaching to a device and/or a recipient.

In some embodiments, aspects of the invention relate to growing organs tethered to a cannula using one or more devices or cannulae having structural features adapted for attachment of the organ during growth. In some embodiments, substitute organs are prepared for testing by growing organs attached to devices or cannulae adapted for connection to pumps, or other mechanical devices. In some embodiments, substitute organs are prepared for transplant by growing the substitute organs attached to devices or cannulae adapted for attachment to the tissue and/or vasculature of a transplant recipient. In some embodiments, organs are prepared for transplant by severing vascular connections below the attachment to growth cannulae in order to provide a clean/healthy vascular tissue for suturing.

Timing Considerations for Modulating Growth Conditions:

In the context of methods for periodically modifying growth conditions, the following parameters may be varied: the amplitude and frequency of changes, the types of conditions that are changed, the types of cues or stimuli that are used to provide a feedback, the duration of the periodic growth conditions, and organ specific factors, as described herein.

In some embodiments, the addition of removal of nutrients, chemicals, waste or any combination thereof can be activated by real or relative time and/or by cues from chemical sensors or mechanical measurements, or any combination thereof.

In some embodiments, at specific times, one or more tissue or mechanical models can be placed into a bioreactor to act as a trigger to stimulate a new phase of reactor organ development. It should be appreciated that the additional tissues may be ones that are naturally associated (e.g., in a natural growth or development context) with the organ or tissue being grown in the reactor. In some embodiments, the additional tissue may be in the form of organs, portions thereof, or tissue fragments. In certain embodiments, the additional tissue may be provided or placed in the same relative position (e.g., with respect to distance and/or three-dimensional orientation) as is found in a natural growth setting. In some embodiments, material (e.g., tissue, organ, other physical objects, growth factors, nutrients, or other molecules described herein) may be provided with a reactor system at the start of organ development (e.g., prior to or when cells are introduced onto a matrix or other support structure). In some embodiments, material may be introduce through a port or other opening as described herein. In some embodiments, material may be present in a volume or storage unit or zone that is connected to the reactor chamber (e.g., within a sterilized system), but only delivered to the growth medium or environment or only deployed to contact the developing tissue or organ in response to a cue, at a predetermined time, or when activated by an operator (e.g., physician or other human). It should be appreciated that any suitable pump, mechanical arm or support, injector, or other pressure (e.g., hydrostatic), or robotic or other mechanism may be used to deliver or deploy the material.

Validation:

In some embodiments, aspects of the invention relate to methods and devices for validating an organ, e.g., to determine that a substitute organ is suitable for transplantation.

In some embodiments, a validation stage may be used during which suitable challenges are provided to test for appropriate organ responses. In some embodiments, the validation stage may be implemented after the maturity stage (e.g., when a substitute organ is fully grown or fully recellularized). In some embodiments, a substitute organ is validated to confirm that it has normal function prior to implantation. In the context of a donor transplant, the surgeon is reassured of the function of the organ because until recently it was functional inside a living human being. However, in the absence of a validation stage, there is no such reassurance that an engineered substitute organ is functional even if it is composed of living cells. Accordingly, tests for one or more structural and/or functional properties may be implemented prior to implantation. In some embodiments, testing may be performed while maintaining sterility and a device may include test components within (e.g., inside) the bioreactor chamber. Non-limiting examples of tests that could be performed on a heart include the following: a heart could be tested for heart rate, blood pressure, ejection fraction etc., prior to implant. In addition to static tests, an organ could be challenged to see if it responds appropriately (e.g., in a dynamic test) to one or more mechanical and/or chemical stimuli (e.g., adrenaline, vasoconstrictors, vasodilators, etc., or any combination thereof).

In some embodiments, lungs could be statically tested for tidal volume and pressure, the pressure volume loop, and/or dynamically tested with a challenge/response to epinephrine or other stimuli. Kidneys could be tested for hormone production (e.g., erythropoietin), compound storage and release (e.g., vitamin D), and/or the normal filtration/dialysis functionality of the kidney (e.g., urea production and the passage of a small molecule drug from the blood to the urine). Livers could be tested for bile production, drug metabolism, enzyme production, etc., and could be challenged with fat or cholesterol to confirm functionality. A pancreas could be tested for insulin production and release in response to glucose. Blood vessels could be tested for response to vasodilators or constrictors. Bladders could be tested for compliance, volume and pressure. Other suitable tests for these and other organs may be included in aspects of the invention.

Accordingly, in some embodiments, a substitute organ may be tested to validate its functional and/or structural properties prior to transplant. In some embodiments, a substitute organ may be challenged with mechanical, physical, neurological, hormonal, enzymatic/chemical, and/or electrical challenges, and/or any combination thereof. In some embodiments, a substitute organ may be challenged over a full range of conditions that the organ will be exposed to in situ.

Accordingly, in some embodiments aspects of the invention relate to a reactor that includes one or more components for stimulating and/or challenging a substitute organ; one or more sensors for monitoring a response to a functional challenge; a support scaffold that incorporates one or more components for stimulating and/or challenging a substitute organ; and/or a support scaffold that includes one or more sensors for monitoring a response to a functional challenge.

In some embodiments, aspects of the invention relate to systems and methods for testing functional properties. In some embodiments, aspects of the invention relate to organ specific testing (e.g., kidney filtration, lung pressure response, O2 saturation profile of lung, etc.).

In some embodiments, a challenge may include testing a response to one or more neurotransmitters; a response to one or more hormonal challenges (e.g., organ-specific hormonal challenges, for example an adrenaline response of a substitute heart, an epinephrine response of a substitute lung); an immunological response (e.g., a response to a challenge with patient specific immunoglobulins); a response to one or more challenges with dietary or other environmental molecules (e.g., organ specific molecules, for example a substitute liver's response to fat, alcohol, or other molecules; or a substitute kidney's ability to filter defined molecules); a response to metabolites; or any combination thereof.

In some embodiments, a profile of metabolites produced by a substitute organ may be determined. In some embodiments, testing may be performed under different conditions (e.g., under typical conditions characteristic of a normal biological or physiological environment at rest, and/or under extreme conditions characteristic of extremes of biological or physiological activity, for example, a deep breath or heavy breathing for a lung, high blood flow/pressure for heart, and the like).

Accordingly, in some embodiments a protocol is provided whereby a substitute organ is grown in the presence of pulsation and/or movement, but the pulsation and/or movement may be halted during functional and/or structural validation.

Sensors:

In some embodiments, aspects of the invention relate to devices comprising one or more sensors. In some embodiments, a sensor may be non-invasive. Accordingly, a bioreactor described herein may optionally include one or more sensors, such as temperature sensors, for determining a component or a condition within a chamber of the bioreactor. For example, one or more temperature sensors may be used to determine the temperature of a fluid inside a chamber or other portion of a bioreactor. One or more pressure sensors may be used to determine the amount of pressure inside a bioreactor. One or more flow rate sensors may determine the flow rate of a fluid flowing in one or more portions of a bioreactor, e.g., so that a particular flow rate can be maintained. In some embodiments, shear stress sensors such as a diverging fringe shear stress sensor or a micro-pillar shear-stress sensor can be used. Sensors for determining components or conditions of a fluid (e.g., nutrient composition and/or concentration, dissolved oxygen concentration, dissolved carbon dioxide concentration, pH, osmolality) may also be incorporated into the bioreactor. A sensor could also be used to measure cell concentration and/or degree or existence of cell adherence to a substrate. One or more sensors (e.g., strain gauges) to determine the weight of the substitute organ may be included in some embodiments. Other sensors may include electrical, pH, ionic, and/or specific chemical sensors as described herein.

In some embodiments, one or more sensors may be deployed within a portion of a device (e.g., within a chamber, conduit, pump, valve, reservoir, sampling zone, etc., or any combination thereof) to detect conditions within the device itself. This information can be indirectly indicative of the status of the developing organ. However, this information may be useful primarily to maintain and/or modify conditions within the reactor depending on the application. In some embodiments, one or more sensors may be deployed directly on a substitute organ or tissue (or a region thereof) in order to directly monitor one or more variables on the organ or tissue.

Flexible Sensors:

In some embodiments, flexible sensors may be used in one or more storage or transport containers or in a bioreactor described herein. Examples of flexible sensors include sensors based on pentacene (a hydrocarbon molecule) and/or carbon nanotubes that may be used to develop temperature sensors and/or strain sensors. In some embodiments, a Wheatstone bridge, an instrument that measures unknown electrical resistance, and a thin pentacene film that acts as a sensing layer may be used to measure strain. Accordingly, physiological strain, such as breathing, that creates a mechanical deformation of the sensor, can be detected as a change in the sensor's resistance to electrical current's. In some embodiments, smaller sensors are more sensitive to current variations. However, sensors of any suitable size or shape may be used.

In some embodiments, a thin-film transistor may be used as a temperature sensor. A thin-film transistor may be developed to provide a linear response to temperature changes within the operating parameters of a device described herein. However, it should be appreciated that any suitable flexible sensor may be used.

In some embodiments, one or more portions of “smart fabric” may be used in contact with an organ or a portion thereof in order to monitor one or more physiological parameters. As used herein, “smart fabric” refers to fabric or other flexible material that includes one or more sensors and/or one or more wireless transmitters and/or other wireless network components and that can be fit onto, around, or over, all or a portion of an organ or tissue in order to provide physiological feedback about the status of the organ or tissue.

Sensor Configurations:

A sensor may be positioned at any suitable location so long as it is operatively associated with the bioreactor. In some cases, a sensor may be located within the organ chamber (e.g., integrated within the wall or on a support structure). In some cases, a sensor may be located within a channel, pipe, and/or reservoir that is connected to the chamber. As such, parameters for monitoring the growth and/or maintenance of cells, tissues, organs, or other entities in the chambers can be determined independently and, in some cases, substantially simultaneously. The one or more sensors may be run continuously, periodically, or in some cases, in response to certain events, such as a threshold level of a nutrient within a liquid in the vessel.

A bioreactor can also include visual aids, such as scales or markers, that can facilitate measurement of the size, length, width of a component in the bioreactor.

A bioreactor may also include a temperature control system for monitoring and/or controlling a temperature of a fluid inside a vessel. The bioreactor may further include a thermocouple and/or a resistance temperature detector for sensing a temperature of the contents inside the vessel. The thermocouple may be operatively connected to the temperature controller to control temperature of the contents in the vessel.

In some embodiments, a sensor may be used for imaging, for example video imaging. In some embodiments, one or more sensors may be integrated into a wall of a chamber, channel, pipe, and/or reservoir. In some embodiments, one or more holographic sensors may be used. In some embodiments, sensors may be integrated into a flexible, disposable “balloon-like” chamber. It should be appreciated that sensors of different types may be useful for monitoring growth; providing information for feedback and/or automated control of growth conditions (e.g., based on chemosensors, temperature sensors, electrical sensors, and/or other sensors); and/or complying with regulatory requirements.

In some embodiments, aspects of the invention relate to reactors that incorporate one or more optical monitoring capabilities (e.g., for monitoring growth conditions, gathering data for regulatory compliance, etc.).

In some embodiments, one or more optical techniques may be used (e.g., nephelometry, near or mid infrared, other forms of radiation described herein, etc.) to detect or monitor organ properties and/or levels of metabolites or waste material in the growth environment. In some embodiments, optical monitoring of a substitute organ and or portions thereof may be used to evaluate one or more structural and/or functional properties of the organ. For example, near IR wavelengths (e.g., 550 and 800 nm) may be used to monitor hemoglobin and oxyhemoglobin in a substitute organ. It should be appreciated that aspects of the invention also relate to optical techniques that can be used to monitor sterility.

In some embodiments, an optical sensor may be located within a bioreactor (e.g., within a chamber). In certain embodiments, a bioreactor may be designed to allow optical analysis from outside the bioreactor. Accordingly, a device or chamber may include one or more fiber optic components adapted for optical analysis at multiple wavelengths; one or more optical components and/or observation “windows” for visual analysis; one or more optical components and/or observation “windows” for near and mid IR (e.g., to analyze gases, chemistries, etc.); one or more optical components and/or observation “windows” for nephelometry. It should be appreciated that any of the windows described herein (e.g., in the context of being transparent to IR, UV, visible, Rahman, or other form of radiation) may be of any suitable size (e.g., about 1-50 mm², 50 mm² to 1 cm², 1-5 cm², 5-10 cm², 10-50 cm², or more or less) or shape (e.g., square, round, rectangular, or any irregular shape) depending on the device and/or detector that is being used.

In some embodiments, aspects of the invention relate to systems and algorithms for gathering, managing, and/or analyzing optical measurements throughout periods of organ growth and maintenance. In some embodiments, aspects of the invention relate to systems and algorithms for gathering, managing, and/or analyzing visual images throughout periods of organ growth and maintenance. In some embodiments, video information may be obtained to evaluate the behavior of a substitute organ (e.g., at rest, in response to challenge, etc.).

In some cases, sensors or other entities associated with a bioreactor are connected to a sensor electronics module (e.g., through wires, wirelessly, optically, etc.), the output of which can be sent to a terminal board and/or a relay box. Various sensors for controlling and/or monitoring one or more process parameters inside the bioreactor such as, for example, temperature, pressure, pH, dissolved oxygen, dissolved carbon dioxide, mixing rate, and gas flow rate, liquid flow rate, can be used. The results of the sensing operations may be input into a computer-implemented control system for calculation and control of various parameters (e.g., temperature and weight/volume measurements) and for display and user interface. Such a control system may also include a combination of electronic, mechanical, and/or pneumatic systems to control heat, air, and/or liquid delivered to or withdrawn from the vessel as required to stabilize or control the environmental parameters of the process operation. It should be appreciated that the control system may perform other functions and is not limited to having any particular function or set of functions.

It should be appreciated that information from one or more sensors that monitor organ or tissue parameters directly may be combined with information from one or more sensors that monitor reactor conditions. The combination of this information may be useful to stage different growth periods, predict the viability and/or functionality of an organ or tissue at an early stage, and or determine when an organ or tissue has grown and developed sufficiently for a transition to a subsequent transport, storage, and/or surgical stage. It should be appreciated that the information or combination of information may be compared to database information (e.g., levels of one or more of the parameters that, alone or in combination, represent one or more of: a transition in the growth stages, a predictor of organ/tissue outcome, a marker of good organ/tissue development, a marker of bad organ/tissue development, an indicator of organ/tissue readiness for transport, storage, or surgery.

The one or more control systems can be implemented in numerous ways, such as with dedicated hardware and/or firmware, using a processor that is programmed using microcode or software to perform the functions recited above or any suitable combination of the foregoing. A control system may control one or more operations of a single chamber of a bioreactor, multiple chambers of a bioreactor, or even multiple (separate or interconnected) bioreactors. The control systems can also be implemented using any of a variety of technologies, including software (e.g., C, C#, C++, Java, or a combination thereof), hardware (e.g., one or more application-specific integrated circuits), firmware (e.g., electrically-programmed memory) or any combination thereof.

In one embodiment, a control system operatively associated with a bioreactor described herein is portable along with the bioreactor itself, and optionally along with any pumps, connectors, and/or sources of fluids. The control system may include, for example, all or many of the necessary controls and functions required to perform a fluidic manipulation (e.g., temperature control, mixing, and performing reactions) in the bioreactor. Advantageously, such a portable control system can be programmed with set instructions, and, if desired, transported (optionally with the bioreactor) and hooked up to the bioreactor, ready to perform a process by an end user. A kit including such and other components may also be provided.

Assessing Tissue and Organ Function or Viability:

In some embodiments, aspects of the invention relate to articles and methods for assessing a condition of at least one portion of a tissue or organ of interest. The articles and methods described herein may provide, in some embodiments, a non-invasive or minimally invasive method of determining a condition of the tissue or organ of interest. In some case, assessment can be performed without the use of labels or contrast agents.

It should be appreciated that methods and devices may be used to assess the function and/or viability of an organ or tissue ex vivo and/or in vivo. Accordingly, uses may include evaluating organs/tissues in bioreactors and/or evaluating organs/tissues in a subject (e.g., to determine whether a transplant is needed, or a site that is appropriate for a transplant, or whether an organ or tissue should be removed and replaced or simply added to). Various conditions can be assessed, such as the viability of a tissue or organ, the determination of a diseased tissue compared to healthy tissue, and/or the monitoring of the growth of a tissue or organ of interest. Advantageously, data on the condition of the tissue or organ of interest may be acquired simply and rapidly using the articles and methods described herein. In some embodiments, the data may be acquired in a consistent and reproducible manner with minimal inter- or intra-observer variation. Furthermore, collection of the data, in some embodiments, does not pose any hazard to the tissue being studied, as determination of the condition of the tissue or organ may take place while the tissue or organ is in a protected or sterile environment. The articles and methods described herein may also allow early, non-subjective detection of diseased cells or tissue, which may increase the likelihood that intervention aimed at saving or healing the tissue will be successful and lead to an improved clinical outcome.

In some embodiments, aspects of the invention relate to interrogating the infrared radiation emanating from a tissue or organ. Each tissue or organ, or portions thereof, may have a natural infrared emission spectrum that may be altered as a result of injury or disease. Accordingly, by detecting and analyzing the infrared radiation emanating from a tissue or organ, indicia of an abnormality (e.g., associated with an injury or disease) may be detected. This information may be used to assist in detecting and/or diagnosing the injury or disease. As described in more detail herein, in some embodiments, the infrared emission associated with an injury or disease may be used to identify a target tissue region and assist in the delivery of a drug, a cell preparation, or other therapy to the target tissue region.

In some embodiments, infrared emission from a tissue or organ may result from the tissue response to forces such as blood flow, air flow, etc., or any combination thereof. In some embodiments, physiological forces in a subject may effect the infrared radiation emanating from tissue or organ structures in the body. In some embodiments, organs grown ex vivo (e.g., in a bioreactor) may have a certain infrared radiation emission spectrum in response to mechanical forces associated with growth in the bioreactor (e.g., fluid pumped through a vasculature, or gas pumped in and out of airways, etc.). This can allow, in some cases, the monitoring of effects of various processes associated with tissue or organ growth and development. The tissues or organs may be grown in situ or in vitro in some cases.

The articles and method described herein may be used to determine a condition of a cell, tissue or organ based on factors such as O₂ intake, temperature, nutritional level, toxin concentration in a solution surrounding the cell, tissue or organ, ion transport through cell membranes. The articles and methods may also be used to determine activity of the cell, tissue or organ by, for example, differentiating chemical concentrations and/or determining energy outputs. Other conditions can also be probed, such as whether the cell, tissue or organ has been defrosted, whether an injected amount of a composition is accurate, whether the cell, tissue or organ is accepting an injected composition, what appropriate volumes of compositions should be injected, and whether an injection device is working appropriately or according to pre-set standards. Other conditions can also be assessed.

In one embodiment, a method of assessing a condition of at least portion of a tissue or organ of interest includes the use of an infrared detector. The method may involve positioning an infrared detector near a tissue or organ of interest, and detecting infrared radiation emanating from at least one portion of the tissue or organ. The method may also involve analyzing the detected infrared radiation and generating data corresponding to the at least one portion of the tissue or organ. In some embodiments, a condition of the least one portion of the tissue or organ can be determined based, at least in part, on the generated data.

In some embodiments, the radiation emanating from a cell, tissue or organ is primarily the result of a natural heat profile of the cell, tissue or organ. For example, the radiation emanated may be primary the result of metabolism associated with cell division, or due to mitochondrial energy production. Detection of radiation emanating from the cell, tissue or organ may produce a spatial heat signature associated with the cell, tissue or organ. Analysis at both the cellular level and the tissue level may be possible using the articles and methods described herein. In some embodiments, the radiation emanating from the tissue or organ is produced in the absence of an imaging agent added to the tissue or organ (although, in other embodiments, an imaging agent may be added to increase the signal detected or for other purposes, as described in more detail herein). A method may involve detecting infrared radiation emanating from a plurality of different portions of the tissue or organ, and comparing any differences between the radiation detected from the plurality of different portions. For example, infrared radiation may be detected from a suspected diseased portion of a tissue or organ and from a suspected healthy portion of the tissue or organ. Differences between the infrared radiation emanating from each of the portions may be determined to help identify or confirm the existence of a healthy and/or a diseased portion. Other metabolic, physiologic and anatomic characteristics of cells, tissues or organs can also be determined.

In some embodiments, a difference between the radiation detected from one portion to another portion of a tissue or organ is primarily due to an increase or decrease in metabolism of cells due to increased or decreased cell division, respectively, from the at least one portion of the tissue or organ. For example, an increase or decrease in metabolism of cells from a first portion of the tissue or organ may generate at least a 0.0001° C. difference, at least a 0.001° C. difference, at least a 0.01° C. difference, at least a 0.1° C. difference, at least a 0.2° C. difference, at least a 0.3° C. difference, at least a 0.4° C. difference, at least a 0.5° C. difference, at least a 0.6° C. difference, at least a 0.7° C. difference, at least a 0.8° C. difference, at least a 0.9° C. difference, or at least a 1.0° C. difference compared to a second portion of the tissue or organ, the difference in temperature corresponding to the difference in radiation detected from the first and second portions. The method may include detecting a difference of less than 0.00001° C., less than 0.0001° C., less than 0.001° C., less than 0.01° C., less than 0.1° C., less than 0.2° C., less than 0.3° C., less than 0.4° C., less than 0.5° C., less than 0.6° C., less than 0.7° C., less than 0.8° C., less than 0.9° C., or less than 1.0° C. between a first portion and a second portion of the tissue or organ of interest. In some embodiments, the increase or decrease in metabolism of cells from a first portion of the tissue or organ generates between a 0.1° C.-0.5° C. difference, between a 0.5° C.-0.1° C. difference, between a 0.1° C.-2.0° C. difference, between a 0.1° C.-5.0° C. difference, or between a 1.0° C.-5.0° C. difference compared to a second portion of the tissue or organ, the difference in temperature corresponding to the difference in radiation detected from the first and second portions. Differences in heat emitted from various portions of the tissue or organ may be the result of, for example, natural cellular heat profiles, blood flow, ATP, respiration and digestion. These and other differences in heat emissions may be detecting using the articles and methods described herein.

In some embodiments, all or an part of the infrared radiation detected from a portion of a tissue or organ is primarily due to an increase or decrease in blood flow to at least one portion of the tissue or organ. For example, an increase in blood flow may lead to the tissue or organ exhibiting higher amounts of infrared radiation compared to another portion having a relatively lower blood flow to the tissue or organ portion. In some embodiments where radiation is detected from at least two different portions of the tissue or organ, a difference in the radiation detected may be the result of an increase or decrease in blood flow to the different portions.

In other embodiments, articles and methods described herein can be used to determine a difference in radiation detected as a result of a change in metabolism in cells (or other cellular processes) that occur in or around the at least one portion of the tissue or organ. For example, radiation detected as result of a change in metabolism of cells may be distinguished from radiation detected as a result of a change in blood flow to the at least one portion of the tissue or organ. Such differences may be determined, in some embodiments, by tuning into specific wavelengths emitted from the tissue or organ. For example, a particular range of wavelengths associated with metabolism of cells may be identified, and these wavelengths may be separated or subtracted from a baseline reference or a different set of ranges of wavelengths associated with another process occurring in or around the tissue or organ portion. These sets of wavelength ranges can be used, in some embodiments, to monitor the growth and/or progress of the tissue or organ, as the metabolism of the cells may change during such processes. In certain embodiments, the determination of these differences may be aided by the use of one or more spectral filters associated with the detector. In certain embodiments, a computer algorithm can help in calculating any such differences. These and other methods can be used to determine a difference between the radiation detected from one portion to another portion of the tissue or organ, wherein the difference is primarily due to cell distress or death. In other embodiments, these and other methods can facilitate determination of when cells are differentiating (e.g., due to an increase in temperature of the cells). In some instances, this information may help monitor the growth of the tissue or organ, e.g., to indicate the particular growth phase of the tissue or organ or to determine when a tissue or organ is healthy and/or ready for implantation or use.

In other embodiments, the articles and methods described herein can be used to analyze and/or map a chemical profile of a tissue or organ. For example, analysis or mapping of concentrations of O₂, CO₂, water, lactic acid, and creatine, as well as ratios of various components such as proteins, lipids, and water can be determined.

As described herein, the methods and articles can be used, in some embodiments, to distinguish a diseased tissue or organ from a healthy tissue or organ. In some cases, the diseased tissue is at a surface of the tissue or organ of interest. In other cases, the diseased tissue is underneath a surface of the tissue or organ of interest. For example, in some embodiments, the diseased tissue is at least 1 mm, at least 15 mm, at least 1 cm, at least 2 cm, at least 3 cm, at least 4 cm, at least 5 cm, at least 6 cm, at least 7 cm, at least 8 cm, at least 9 cm, at least 10 cm, at least 12 cm, at least 15 cm, or at least 20 cm underneath a surface of the tissue or organ of interest. In other embodiments, the diseased tissue is less than 1 mm, less than 15 mm, less than 1 cm, less than 2 cm, less than 3 cm, less than 4 cm, less than 5 cm, less than 6 cm, less than 7 cm, less than 8 cm, less than 9 cm, less than 10 cm, less than 12 cm, less than 15 cm, or less than 20 cm underneath a surface of the tissue or organ of interest. Detection of diseased tissue at other locations is also possible.

It should be understood that a variety of different tissues or organs can be analyzed using the methods and articles described herein. In some embodiments, the tissue or organ of interest is one of an adrenal gland, an appendix, a bladder, a brain, a breast, a colon, an eye, a gall bladder, a heart, an intestine, a kidney, a liver, a lung, an esophagus, a larynx, an ovary, a pancreas, a parathyroid, a pituitary gland, a prostate, a skin, a spleen, a stomach, a testicle, a thymus, a thyroid, a trachea, a uterus, a urethra, a ureter, an artery, and a vein. Other tissues or organs of interest are also possible. In some cases, the tissue or organ of interest is one of regenerative tissue, confluent cells, or a morphological feature with no visible contrast. Advantageously, the methods and articles described herein can be used to determine differences in such cells or tissues non-invasively, or minimally invasively, in some embodiments.

In one particular embodiment, the methods and articles described herein can be used to distinguish an infarcted tissue or organ from a healthy tissue organ. Certain existing methods of diagnosing a heart attack or susceptibility of a heart attack, or locating infarcted tissue, involve injecting dyes or other components into the heart and determining where such dyes are located within the tissue. Other methods may involve a surgeon physically feeling the surface of the heart and determining where the differences in the feel of the tissue exist (e.g., due to differences in hardness, elasticity, or other physical properties) between healthy and diseased tissue. In some cases, this can be combined with a visual determination of any color or other physical change between different portions of the tissue. Although such methods are possible, often they are subjective, may involve use of components that may adversely affect the health of the patient, and/or may have side effects. To circumvent these and other potential problems, the articles and methods described herein can be used to distinguish an infarcted tissue or organ from a healthy one. Methods and articles described herein may also be used to determine, in some embodiments, a disease condition of a patient comprising the tissue or organ of interest. For example, the disease condition may be cancer or other conditions described herein.

The tissue or organ of interest may be positioned at any suitable location during analysis. In some embodiments, the tissue or organ of interest is in-vivo. In some cases, the tissue or organ is surgically exposed. In other embodiments, the tissue or organ of interest is ex-vivo. For example, the tissue or organ of interest may be positioned in a bioreactor, such as a bioreactor described herein. In some embodiments, the bioreactor may be suitable for growing a tissue or organ (e.g., a whole organ) in a bioreactor and monitoring the growth of the tissue or organ using the articles and methods described herein. For instance, determining a condition of at least one portion of the tissue or organ may include determining whether the tissue or organ is developing normally or abnormally. In addition to the size and shape of the tissue or organ, the cellular activity of the cells making up the tissue or organ may be analyzed. Accordingly, the overall health of the tissue or organ can be determined.

In some cases, diseased or unhealthy portions of a tissue or organ can not only be identified, but can also be treated. For example, a therapeutic agent such as a drug, stem cells, or other components known in the art can be delivered (e.g., topically, injected, or by other means) to the identified diseased portion. Other treatments are also possible.

As described herein, methods of assessing the condition of a tissue or organ of interest may involve the use of infrared radiation. The infrared radiation may be, for example, short-wavelength infrared radiation, mid-wavelength infrared radiation, long-wavelength infrared radiation, or far-infrared radiation.

In certain embodiments, the infrared radiation detected has a wavelength of, for example, between 700 nm and 1400 nm, between 1400 nm and 3000 nm, between 3000 nm and 8000 nm, between 8000 nm and 15000 nm, or between 15000 and 1 mm. In certain embodiments, the infrared radiation detected has a wavelength of, for example, between 700 nm and 1000 nm, between 1000 nm and 3000 nm, between 3000 nm and 5000 nm, between 8000 nm and 12000 nm, between 7000 nm and 14000 nm, or between 12000 and 30 mm. Accordingly, in some embodiments one or more transparent regions may be transparent to one or more of the wavelengths described above.

The infrared radiation is detected may be detected using any suitable detector. In some embodiments, the detector comprises a detecting element comprising silicon, doped-silicon, InGaAs, InSb, HgCdTe, PbSe, or a combination thereof.

In addition to measuring infrared radiation, in some embodiments, articles and methods described herein can be used to detect radiation from the visible range emanating from the at least one portion of the tissue or organ. The portion of the tissue or organ of interest analyzed using visible light may be the same portion (or a different portion) analyzed using infrared radiation. In some embodiments, analysis includes detecting radiation from the visible range emanating from the at least one portion of the tissue or organ and generating data corresponding to the at least one portion of the tissue or organ. The plurality of different portions of the tissue or organ may be analyzed, and the differences between the radiation detected from the plurality of different portions can be compared with one another. In such a manner, the radiation from the visible range can be collected into a single image. In some cases, data from the visible range can be combined with data from the infrared range into a single image. For example, the visible and infrared radiation data may be superimposed with one another into a single image.

In certain embodiments, the pressure of the at least one portion of the tissue or organ can be detected. The detected pressure may be analyzed and data may be generated corresponding to the at least one portion of the tissue or organ. Pressure from a plurality of different portions of the tissue or organ may be detected, and any differences between the pressure detected from the plurality of different portions can be compared. In some embodiments, the pressure data can be combined with data from the infrared radiation, data from the visible range, and/or other data, e.g., into a single image.

In certain embodiments, the temperature of the at least one portion of the tissue or organ can be detected. The detected temperature may be analyzed and data may be generated corresponding to the at least one portion of the tissue or organ. Temperature from a plurality of different portions of the tissue or organ may be detected, and any differences between the temperature detected from the plurality of different portions can be compared. In some embodiments, the temperature data can be combined with data from the infrared radiation, data from the visible range, pressure data, and/or other data, e.g., into a single image.

In certain embodiments, vibration analysis of the at least one portion of the tissue or organ can be performed. The vibrational analysis may be analyzed and data may be generated corresponding to the at least one portion of the tissue or organ. Additionally, vibrational analysis may be performed with a plurality of different portions of the tissue or organ, and any differences between the vibrational analysis from the plurality of different portions can be compared. In some embodiments, the data from the vibrational analysis can be combined with data from the infrared radiation, data from the visible range, pressure data, temperature data, and/or other data into a single image.

In some embodiments, a fluorescent label (or any other suitable label) can be added to the tissue or organ of interest, and fluorescence (or other emission) can be detected from the at least one portion of the tissue or organ. The fluorescence detected may be analyzed, and data may be generated corresponding to the at least one portion of the tissue or organ. Additionally, fluorescence emission may be detected from a plurality of different portions of the tissue or organ, and any differences between the fluorescence emission between the plurality of different portions can be compared. In some embodiments, the fluorescence data can be combined with data from the infrared radiation, data from the visible range, data from the vibrational analysis, pressure data, temperature data, and/or other data into a single image.

In some embodiments, a non-visible contrast agent (or any other suitable agent) can be added to the tissue or organ of interest, and emission from the non-visible contrast agent can be detected from the at least one portion of the tissue or organ. The emission from the non-visible contrast agent may be analyzed, and data may be generated corresponding to the at least one portion of the tissue or organ. Additionally, emission from the non-visible contrast agent may be detected from a plurality of different portions of the tissue or organ, and any differences between the emission between the plurality of different portions can be compared. In some embodiments, the non-visible contrast agent data can be combined with data from the infrared radiation, data from the visible range, data from the vibrational analysis, pressure data, fluorescence data, temperature data into a single image. and/or other data into a single image.

In certain embodiments, Raman analysis of the at least one portion of the tissue or organ can be performed. The radiation detected from the Raman analysis may be analyzed and data may be generated corresponding to the at least one portion of the tissue or organ. Additionally, Raman analysis may be performed with a plurality of different portions of the tissue or organ, and any differences between the Raman analysis from the plurality of different portions can be compared. In some embodiments, the data from the Raman analysis can be combined with data from the infrared radiation, data from the visible range, data from the vibrational analysis, pressure data, fluorescence data, temperature data, and/or other data into a single image.

In certain embodiments, absorbance, transmission and/or reflectance from the at least one portion of the tissue or organ can be performed. The absorbance, transmission and/or reflectance may be analyzed and data may be generated corresponding to the at least one portion of the tissue or organ. Additionally, absorbance, transmission and/or reflectance can be detected from a plurality of different portions of the tissue or organ, and any differences between the absorbance, transmission and/or reflectance from the plurality of different portions can be compared. In some embodiments, the data from the absorbance, transmission and/or reflectance detection can be combined with data from the infrared radiation, data from the visible range, data from the vibrational analysis, pressure data, fluorescence data, temperature data, and/or other data into a single image.

In some cases, articles and methods described herein can be used to detect portions of the tissue or organ of interest that absorb lipids or other chemicals. Other chemical analyses and detection can also be performed. Chemical analysis may be combined with other detection methods described herein. In some cases, non-visible (e.g., near-infrared) and visible anatomical features, chemical distribution and location, and physiological and metabolic spatial analysis and location of activities can be displayed on a single image, or multiple images.

In certain embodiments, electrical analysis of the at least one portion of the tissue or organ can be performed. The electrical analysis may involve, for example, measuring the electrical potential of the tissue or organ. In some cases, an electrical potential is first applied to the tissue or organ (e.g., using an electrode or other probe), and a response from the tissue or organ is detected. The electrical analysis may be analyzed and data may be generated corresponding to the at least one portion of the tissue or organ. Additionally, electrical analysis may be performed with a plurality of different portions of the tissue or organ, and any differences between the electrical analysis from the plurality of different portions can be compared. In some embodiments, the data from the electrical analysis can be combined with data from the infrared radiation, data from the visible range, data from the vibrational analysis, pressure data, fluorescence data, temperature data, data from the Raman analysis, absorbance, transmission and/or reflectance analysis, and/or other data into a single image.

In some cases, determination of a condition of tissue or organ involves superimposing (e.g., overlaying) two or more data sets into a single image. For example, the method may include superimposing two or more of the infrared data, data from the visible range, temperature data, pressure data, data from vibrational analysis, data from the Raman analysis, and/or fluorescence data into a single image. Such and other images may be overlaid in real time. Additionally or alternatively, one set of data or an image may be superimposed with a second set of data or image such as an X-ray image, a MRI image, a CAT scan image, a positron emission tomography image, an ultrasound image, and/or a single photon emission computer tomography image. The data or images can be superimposed into a single image, or into multiple images, the specific combination of which may be chosen by the user.

In some cases, the single image or the multiple images is a real-time image(s). The one or more images may be converted into a negative image, a black and white image, a color image, and/or a color-coded image. The images may be recorded, and in some embodiments, may be simultaneously displayed and recorded.

In some embodiments, a normal and/or diseased and/or defective profile (e.g., image(s)) may be defined in comparison to a known normal profile (e.g., image(s)). The known normal profile may be a standard reference profile for a normal tissue or organ. In some embodiments, a subject may be scanned to obtain a personalized reference for one or more healthy organs and or tissues (provided the organs or tissues are healthy in the subject at the time of the reference analysis). This healthy reference may be stored as part of the patient medical records used for comparison to profiles obtained during subsequent evaluations. Changes in infrared profiles, vibration profiles, heat profiles, profiles based on other parameters described herein, other physical properties, or any combination thereof, at one or more locations within a tissue or organ may be used to identify diseased regions or may be used as an initial screen to identify tissue or organs that need to be evaluated using additional techniques in order to determine their status, and/or to identify and/or evaluate tissues/organs required for growth or transplantation.

As such, a determining step may include comparing the data generated (from the parameters described herein) with data (e.g., of the same parameter type) from the portion of the tissue or organ collected on a prior occasion. This may involve, in some embodiments, comparing the data generated with reference data from a tissue or organ of similar type and/or condition. In some cases, a determining step involves comparing the data generated with reference data from a healthy tissue or organ of similar type. For example, the tissue or organ of interest may be one from a first patient, and the determining step may include comparing the data generated with data from a tissue or organ of a second patient. In other embodiments, the tissue or organ of interest is one of a first patient, and the determining step includes comparing the data generated with data from a tissue or organ of the first patient. Other comparisons are also possible.

As described herein, one or more parameters of a tissue or organ can be detected. In some such embodiments, a detector for each parameter can be present in a single detecting unit. This can allow detection of the different parameters to take place simultaneously in some embodiments. Certain detectors for detecting the parameters described herein are known in the art and can be used in embodiments described herein. For example, in some embodiments, the detector is a high resolution infrared detector. In other embodiments, detection involves the use of a stereoscopic image detector. The stereoscopic image detector, or other detection unit described herein, may include at least two detectors. The at least two detectors may be focused simultaneously on at least one portion of the tissue or organ of interest. In certain embodiments, at least 2, at least 5, at least 10, at least 25, at least 50, at least 100, at least 200, at least 500, at least 1,000, at least 5,000, or at least 10,000 detectors is used. The detectors may be part of a single detection unit, or multiple detection units. In some cases, a two-dimensional or three-dimensional array of detectors is used.

The number of detectors used may depend, at least in part, on the number of portions of the tissue or organ of interest to be analyzed, the area of the portions to be analyzed, and/or the distance of the portions to be analyzed. As described herein, a method may involve detecting infrared or other radiation emanating from a plurality of portions of a tissue or organ of interest. The plurality of portions may comprise, for example, at least 2, at least 5, at least 10, at least 25, at least 50, at least 100, at least 200, at least 500, at least 1,000, at least 5,000, or at least 10,000 different portions of the tissue or organ. In some cases, the plurality of portions comprises less than 2, less than 5, less than 10, less than 25, less than 50, less than 100, less than 200, less than 500, less than 1,000, less than 5,000, or less than 10,000 different portions of the tissue or organ. Each portion may comprise an area of, for example, at least 1 nm², at least 10 nm², at least 100 nm², at least 1 μm², at least 10 μm², at least 100 μm², at least 1 mm², at least 10 mm², at least 100 mm², or at least 1 cm². In some cases, each portion comprises an area of, for example, less than 1 nm², less than 10 nm², less than 100 nm², less than 1 μm², less than 10 μm², less than 100 μm², less than 1 mm², less than 10 mm², less than 100 mm², or less than 1 cm². The distance between adjacent portions may be, for example, at least 1 nm, at least 10 nm, at least 100 nm, at least 1 μm, at least 10 μm, at least 100 μm, at least 1 mm, at least 10 mm, at least 100 mm, at least 1 cm, at least 5 cm, or at least 10 cm. In some cases, a distance between adjacent portions is, for example, less than 1 nm, less than 10 nm, less than 100 nm, less than 1 μm, less than 10 μm, less than 100 μm, less than 1 mm, less than 10 mm, less than 100 mm, less than 1 cm, less than 5 cm, or less than 10 cm. Suitable numbers and types of detectors can be used based on the parameters described above and herein for a particular application.

It should be appreciated that one or more detectors or sensors described herein may be integrated into a device (e.g., into the wall of a device) to i) monitor and/or evaluate a substitute tissue or organ and/or ii) to monitor and/or evaluate conditions within a device (e.g., within a chamber or other component of a device).

Detection can be performed using one or more detectors positioned at any suitable location with respect to the tissue or organ of interest. In some embodiments, the detecting step is performed in the absence of an endoscope. The one or more detectors may be operatively associated with a bioreactor (e.g., integrally connected to the bioreactor) in some embodiments. For example, a tissue or organ may be positioned between a detector and a source of radiation or other energy in some instances. In other embodiments, one or more detectors is positioned on a head-mounted device, as described in more detail below. In some cases, one or more detecting steps are performed while one or more detectors is positioned at least 1 mm, at least 1 cm, at least 5 cm, at least 10 cm, at least 20 cm, at least 50 cm, at least 1 m, at least 2 m, at least 3 m, or at least 5 m away from the tissue or organ of interest. In other embodiments, one or more detecting steps are performed while one or more detectors is positioned less than 1 mm, less than 1 cm, less than 5 cm, less than 10 cm, less than 20 cm, less than 50 cm, less than 1 m, less than 2 m, less than 3 m, or less than 5 m away from the tissue or organ of interest.

In addition to one or more detectors, a method or article described herein may include a source of radiation or other energy that is applied to the tissue or organ of interest. In some cases, radiation or other energy applied is in the form of infrared radiation, near-infrared radiation, ultraviolet radiation, near-ultraviolet radiation, radiation from the visible range, heat, pressure, or an electrical potential. As described herein, a method may include applying radiation or other energy to the at least one portion of the tissue or organ, and then detecting radiation or other energy emanating from at least one portion of the tissue or organ. These and other methods can be used to determine a condition of the tissue or organ of interest.

One or more sources of radiation or other energy may be positioned at any suitable location with respect to the tissue or organ of interest. The one or more sources of radiation or other energy may be operatively associated with a bioreactor (e.g., integrally connected to the bioreactor) in some embodiments. In other embodiments, one or more sources of radiation or other energy is positioned on a head-mounted device, as described in more detail below. In yet other embodiments, one or more sources of radiation or energy is positioned (e.g., removably positioned or integrally positioned) within a tissue or organ of interest. For example, a source of radiation (e.g., visible, infrared, ultraviolet or other forms of radiation described herein) may be positioned within a hollow portion of a tissue or organ, allowing the radiation to emit from an inside portion to an outside portion of the tissue or organ. In some cases, the radiation source may be part of a support (e.g., a scaffold) for supporting the tissue or organ. This configuration may be useful, for example, for visualizing structures such as vasculature, blood flow, as well as cell, tissue, and organ health. One or more detectors positioned at an outside portion of the tissue or organ can be used to detect the radiation emitted from the tissue or organ.

In some cases, one or more detecting steps are performed while one or more sources of radiation is positioned at least 1 mm, at least 1 cm, at least 5 cm, at least 10 cm, at least 20 cm, at least 50 cm, at least 1 m, at least 2 m, at least 3 m, or at least 5 m away from the tissue or organ of interest. In other embodiments, one or more detecting steps are performed while one or more sources of radiation is positioned less than 1 mm, less than 1 cm, less than 5 cm, less than 10 cm, less than 20 cm, less than 50 cm, less than 1 m, less than 2 m, less than 3 m, or less than 5 m away from the tissue or organ of interest.

In some embodiments, a detector/sensor may be positioned on the outside of a reactor chamber, but the distance between the detector/sensor and a substitute organ or tissue within the chamber may be changed, for example, by moving the support structure and the substitute organ or tissue towards the wall next to the detector/sensor, or by having a flexible portion of the wall that allows the detector/sensor to be pushed close to (or up against) the substitute organ or tissue on the support structure. In some embodiments, the distance separating the surface of the substitute organ or tissue and the detector/sensor (e.g., IR detector) is about a ¼ inch or less (e.g., from about ¼ to ⅛, from about ⅛ to 1/16, or less). In some embodiments, this distance is the distance between the tissue or organ surface and the inside of the wall (and this distance covers a fluid containing zone, e.g., saline in which the tissue or organ is growing). In some embodiments, this distance also includes the thickness of the wall of the chamber. It should be appreciated that the wall of the chamber may be transparent to the wavelength being detected (at least within the region that is being used to detect signal from the organ or tissue).

In some embodiments, the radiation or other information detected from the plurality of portions may be used to form a two-dimensional or three-dimensional map. The map may include, for example, a standard reference frame including one or more reference points (or reference lines). The reference point(s) or line(s) may correlate with, for example, a specific, identifiable portion of the tissue or organ of interest. For example, for a brain, skull landmarks such as bregma, lambda, and the interaural line, are commonly used as the origins of a coordinate system. Similar landmarks may be identified with the tissue or organ of interest to form one or more reference points (or lines) to generate a standard reference frame which may be specific to the type, age, and/or organism inhabiting the tissue or organ of interest. The map may also include coordinates that can allow determination of locations of each of the different portions of the tissue or organ on the map. The standard reference frame may be displayed along with the one or more images described herein (e.g., superimposed images).

It should be appreciated that the images and/or standard reference frame may be displayed using any suitable technique. In some embodiments, different thresholds may be set and different levels of the parameter being measured may be represented using different colors and/or intensities. In some embodiments, the images may be superimposed with one or more different images (e.g., images described herein such as visual images, reconstructed images, heat profiles, etc., or any combination thereof) to provide additional functionality or information. In some embodiments, certain combinations of infrared emission and other properties described herein may be used for diagnostic purposes. For example, an abnormal infrared radiation profile in combination with an abnormal heat profile may identify a organ or tissue region as diseased or injured with greater statistical significance than either profile alone.

One or more images may be displayed on any suitable display unit. In some cases, one or more images is displayed on a head-mounted display unit, an orthogonal view display unit, a cathode ray tube unit, an autostereoscopic display unit, a volumetric display unit, or a liquid crystal display unit. The image(s) displayed may be, for example, an orthogonal projection, e.g., using the data generated as described herein.

In some embodiments, aspects of the invention relate to a head-mounted device for displaying images, data, and/or other observable features of the tissue or organ of interest. The head-mounted device may include one or more of the features described above and herein. For example, in one particular embodiment, the head-mounted device may include a strap, two displays, one or more detectors (e.g., cameras or other detectors) connected to each display, and other components. A first detector may be operatively associated with a right display and a second detector may be operatively associated with a left display (e.g., one for each eye). It should be understood, however, that other configurations are possible.

In some embodiments, a head-mounted device includes a display that can be used to overlay or superimpose information (e.g., images) from different detectors. In some embodiments, two of more of the following types of information can be overlaid: a visual image, an infrared image, a Raman image, a pressure image, a temperature image, a vibrational analysis image, a fluorescence image, an image associated with emission from a non-visible contrast agent, an image from electrical analysis, and/or additional information. Such and other images may be overlaid in real-time. Additionally or alternatively, one or more images may be superimposed with one or more images that were taken of the tissue or organ of interest at an early point in time. Such images include, for example, an ultrasound image, an X-ray image, a MRI image, a CAT scan image, a positron emission tomography image, and/or a single photon emission computer tomography image. The data or images can be superimposed into a single image, or into multiple images, the specific combination of which may be chosen by the user.

In some embodiments, a head-mounted device may include two or more displays to provide a stereo image to the user. Each display may overlay two or more types of information as described above.

As described herein, different numbers and types of detectors may be operatively associated with the head-mounted device. Thus, the detecting and displaying steps described above and herein can be performed with the head-mounted device. In some embodiments, the detecting, displaying, as well as analyzing steps can all be performed with the same head-mounted device. In some instances, the head-mounted device includes two ore more detectors that allows an orthogonal viewing ability.

In some cases, a detector (e.g., a camera, such as a video camera) has an auto-focus ability (e.g., a depth perception auto-focus ability) with a sufficient dynamic range to allow the user to move his/her head and detect magnified information from the tissue, but also observe surrounding material and areas with lower magnification. The auto-focus ability may be performed in real-time. For example, the head-mounted device may be a what-you-see-is-what-you-get (WYSIWYG) optical viewing system. This can allow the user to operate other tools, whether they be surgical instruments, controller, or physical observations of other displays (e.g., monitors) or other parts of a patient being operated on or instrument being used. Certain detectors known in the art which may provide dynamic range and auto-focus ability may be used in embodiments described herein.

As noted above, the head-mounted device may include a microscope or other suitable magnification unit. The device may have, for example, at least a 10×, at least a 15×, at least a 20×, at least a 50×, at least a 100×, at least a 250×, or at least a 500× magnification ability. comprising monitoring an event within a cell of the at least one tissue or organ of interest. In some embodiments, this magnification ability can allow the device to be used for applications such as monitoring a binding event within a cell of the at least one tissue or organ of interest. In some cases, it can be used to monitor events within a plurality of cells of at least one tissue or organ of interest.

In some embodiments, the head-mounted device comprises a binocular telescope. The device may have, for example, at least a 10×, at least a 15×, at least a 20×, at least a 50×, at least a 100×, at least a 250×, or at least a 500× reduction (e.g., demagnification) ability. In certain embodiments, the device comprises both a microscope and binocular telescope.

The head-mounted device may have other characteristics described herein, such as a source of radiation (e.g., infrared, ultraviolet, and/or other radiation described herein) such that radiation to at least one portion of the tissue or organ is emitted from the device. The device may also include a spectral filtering ability as described herein.

It should be appreciated that a head-mounted device may be powered using any suitable power source (e.g., one or more batteries, a wired connection to a power source, etc., or any combination thereof). It should be appreciated that any suitable power source, e.g., providing alternative and/or direct current, may be used.

In some embodiments, the head-mounted device includes a controller (e.g., a computer) and/or software, which may be incorporated into the device. In some embodiments, the head-mounted device may be controlled by a remote computer and information may be transmitted via a wire or wirelessly.

In some embodiments, aspects of the head-mounted device may be user-controlled. Controls may be operated using any suitable technique. In some embodiments, controls may be mounted on the device, allowing the operator to control with one or both hands. In some embodiments, controls may be voice-activated. In some embodiments, controls may be hand-held and/or foot-operated. Hand or foot controls may relay a signal to the head-mounted device via a wire, wirelessly, or a combination thereof for different functions being controlled. In some embodiments, controls may be located at a remote position and operated by a second individual who communicate with the user of the head-mounted device. The head-mounted device may also include an image stabilization control ability.

In one example, a control (such as a foot-operated control) allows a user to alter one or more parameters such as the magnification, which information to overlay (e.g., infrared, visible, vibrational, temperature, pressure, fluorescence, electrical, or other information described herein), while having free hands to operate other devices or instruments or to operate on a patient (e.g., within the body of the patient) or manipulate an organ or tissue within a bioreactor (using one or more sterile techniques described herein). In one embodiment, the user (e.g., a surgeon) may focus on the tissue or organ of interest and determine that an infrared emission would be helpful in determining the location of veins and arteries in an organ of interest (e.g., since the veins and arteries may have specific infrared profiles that show decreased or increased emission compared to other parts of the organ). The user could choose all or portions of the organ for targeting the detection of infrared emission data. The data can be analyzed using software within the head-mounted device, and the data generated into a two- or three-dimension image in a viewing display. At the same time, visible radiation can be detected, showing normal viewing of the organ. This data can be optionally analyzed, and then generated into a two- or three-dimensional image. If desired, the infrared and visible radiation images can be superimposed into a single image, which can allow the user to see locations of structures (e.g., veins and arteries) that the user could not have easily seen by emission in the visible spectrum. In some cases, the superimposed image can be viewed in real-time, and any adjustments by the user can be seen in the superimposed image. For example, the user could control the magnification of the superimposed image to focus in on certain portions of the organ of interest during an evaluation or operation. The overlay of information can be used to identify areas for surgical or other intervention based on a combination of types of information. When the user looks away from the organ of interest, e.g., to obtain tools or other components for the operation, the auto-focus ability of the device may allow for instantaneous change in depth perception. Other modes of operation are also possible and envisioned within the context of the invention.

Accordingly, the head-mounted device may be used in a variety of different applications. In some embodiments, information from the user-mounted device may be combined with other information described herein to provide feedback for one or more growth transitions or other decisions described herein. However, it should be noted that a head-mounted device described herein may be used for other applications. In some embodiments, the device is adapted and arranged to be worn by a surgeon, who can use the device to perform surgery (e.g., heart surgery, incisions, injections, sutures, detectors, and/or any other interventions where enhance observations are useful, or surgery on other organs described herein). In other embodiments, the device is adapted and arranged to be worn by a phlebotomist, who can use the device to collect blood from a patient comprising the tissue or organ of interest. In yet other embodiments, the device is adapted and arranged to be worn by a dentist. Other examples of non-limiting applications where the head-mounted device can be used include animal research, clinical surgery (e.g., operating room loop replacement and surgical microscope replacement), industrial quality control (e.g., real-time product quality control packaging inspection), low vision conditions (e.g., to enhance vision), medical applications (e.g., skin and throat visualization, detection of skin lesions), process control quality control (e.g., pipe or weld inspection, circuit inspection, regenerative organs, tissue engineering, detection/identification of the chemical makeup of surfaces or contaminants in a container), security/forensics/armed forces (e.g., crime scene investigation, factory surveillance), semiconductor industry (e.g., silicon inspection, board quality control), sports (e.g., sports games).

As described herein, the head-mounted device may used to enhanced images: not only visual but combine visual images with other types of information. In some cases this provides a simple enhancement to allow a user to identify features that are not visually observable (e.g., heat profiles, vibration profiles, etc.). This allows a user to determine areas of diseased or otherwise abnormal tissue for any suitable application (e.g., for a surgical intervention). In some embodiments, enhanced images may be provided by algorithms that combine different types of information and provide new signals based on combinations of features that are shown to be clinically or physiologically relevant where any one of the individual types of information would not be sufficient. The novel information could be displayed in any fashion. For example, different colors could be used to display different properties of tissue (for example, a combination of information that is normal may be displayed in a first color, for example green, whereas a combination of information that is below or above a threshold for an abnormal tissue may be displayed in a second color, for example red). It should be appreciated that additional thresholds and/or alternative information may be provided using additional or alternative colors.

The head-mounted device may have one or more of the following benefits: lower cost vs. higher capability than traditional stereo bench scopes; depth of field may be better than regular optics since you do not need to be close to the object to have a large magnification factor and can change view infinitely; viewing angle change with head movement so no sophisticated stage, or balancing hardware required; small, light for portability and long-term use; can record what is seen; can display and record simultaneously, e.g., for teaching, mimicking SOP's; multiple modes: negative, black and white, color, infrared, ultraviolet, temperature; battery or wall powered allowing for remote viewing.

In some embodiments, articles and method described herein for assessing a condition of at least one portion of a tissue or organ of interest further comprises the step of growing the tissue or organ of interest in a bioreactor. Additionally or alternatively, the detection methods described herein may be used to visualize a tissue or organ of interest which is positioned in a bioreactor.

In some embodiments, a head-mounted device is used to visualize a tissue or organ positioned in a bioreactor. In other embodiments, a detection device is operatively associated with a bioreactor for growing the tissue or organ of interest. For example, the device may be integrally connected to a bioreactor for growing the tissue or organ of interest.

Detection of at least a portion of a tissue or organ of interest that is positioned in a chamber of a bioreactor may involve, in some embodiments, the use of a deformable structure (e.g., a pouch) that surrounds or supports all or a portion of the tissue or organ of interest, as described herein. The deformable structure may be formed of a material that is transparent or semi-transparent to the radiation used to interrogate the tissue or organ. In some embodiments, the deformable structure is held in physical contact with the tissue or organ so that little or no liquid remains between the organ and the liquid in the chamber. For instance, a vacuum may be applied so as to create a tight seal between the organ and the liquid. In one particular set of embodiments, after applying the deformable structure to the tissue or organ (e.g., which may occur in a sterile environment when the tissue or organ is positioned in the chamber), liquid from the chamber may be removed. This may be useful for certain detection techniques used to interrogate the tissue or organ of interest, such as certain infrared detection techniques, since water absorbs infrared light. The deformable structure may also allow a surgeon or other user to feel the tissue or organ before deciding on whether to transplant the tissue or organ. In other cases, the deformable structure may allow ultrasound imaging where the sound source needs to be in physical contact with the surface of the tissue or organ. It should be understood, however, that other detection techniques (e.g., detection of absorbance, transmission, and/or reflection) may also benefit from the use of a deformable structure. The deformable structure may also be useful in allowing the tissue or organ to be inspected at close range (e.g., close enough that a microscope could be used to examine the tissue or organ at the cellular level, or the level of the vascular for instance).

In certain embodiments, assessing a condition of at least one portion of a tissue or organ of interest involves positioning an energy transfer device between the tissue or organ of interest and the detector. As described herein and in more detail in PCT/US2010/002595, the contents of which are incorporated herein by reference in their entirety for all purposes, the energy transfer device may directionally promote energy transfer into or out of the tissue or organ of interest. The insertable member may comprise, for example, an optical fiber or other suitable light-directing component. The energy transfer device can be used, in some instances, to promote transfer of a first range of wavelengths and impede transfer of a second range of wavelengths.

In some cases, the energy transfer device comprises an insertable member or a plurality of insertable members that can be inserted into, onto, or through a surface of the tissue or organ of interest to provide a pathway for energy transfer. The energy transfer device may comprise, for example, a linear or two-dimensional array of insertable members. The plurality of insertable members may include, for example, at least 2, at least 5, at least 10, at least 25, at least 50, at least 100, at least 200, at least 500, at least 1,000, at least 5,000, or at least 10,000 insertable members. Each of the plurality of insertable members may include, for example, a cross-section area of at least 1 pm², at least 10 pm², at least 100 pm², at least 1 nm², at least 10 nm², at least 100 nm², at least 1 μm², at least 10 μm², at least 100 μm², at least 1 mm², at least 10 mm², at least 100 mm², or at least 1 cm² that is inserted into, onto, or through a surface of the tissue or organ of interest to provide a pathway for energy transfer. In some cases, each of the plurality of insertable members has a cross-section area of, for example, less than 1 pm², less than 10 pm², less than 100 pm², less than 1 nm², less than 10 nm², less than 100 nm², less than 1 μm², less than 10 μm², less than 100 μm², less than 1 mm², less than 10 mm², less than 100 mm², or less than 1 cm² that is inserted into, onto, or through a surface of the tissue or organ of interest to provide a pathway for energy transfer.

An average distance between adjacent insertable members may be, for example, at least 1 nm, at least 10 nm, at least 100 nm, at least 1 μm, at least 10 μm, at least 100 μm, at least 1 mm, at least 10 mm, at least 100 mm, at least 1 cm, at least 5 cm, or at least 10 cm. In some cases, an average distance between adjacent insertable members is less than 1 nm, less than 10 nm, less than 100 nm, less than 1 μm, less than 10 μm, less than 100 μm, less than 1 mm, less than 10 mm, less than 100 mm, less than 1 cm, less than 5 cm, or less than 10 cm. An average length of the insertable members may be, for example, at least 1 nm, at least 10 nm, at least 100 nm, at least 1 μm, at least 10 μm, at least 100 μm, at least 1 mm, at least 10 mm, at least 100 mm, at least 1 cm, at least 5 cm, or at least 10 cm. In some cases, an average length of the insertable members may be, for example, less than 1 nm, less than 10 nm, less than 100 nm, less than 1 μm, less than 10 μm, less than 100 μm, less than 1 mm, less than 10 mm, less than 100 mm, less than 1 cm, less than 5 cm, or less than 10 cm.

In some embodiments, aspects of the invention relate to interrogating the vibrational properties of a tissue or organ. According to aspects of the invention, each tissue or organ has natural vibrational properties that may be altered as a result of injury or disease. Accordingly, by detecting and analyzing vibrational properties of a tissue or organ, indicia of an abnormality (e.g., associated with an injury or disease) may be detected. This information may be used to assist in detecting and/or diagnosing the injury or disease. In some embodiments, vibrational properties associated with an injury or disease may be used to identify a target tissue region and assist in the delivery of a drug, a cell preparation, or other therapy to the target tissue region.

In some embodiments, vibrational properties of a substitute organ grown in a bioreactor may be detected and evaluated to determine the status of the organ. In some embodiments, the vibration profile of a substitute organ or portion thereof may be compared to a reference profile known to represent a functional organ that is acceptable for transplantation. In some embodiments, the vibration profile of a substitute organ or portion thereof may be compared to a reference profile known to represent an organ that is not acceptable (e.g., functionally or structurally) for transplantation (e.g., either because the growth and development of the substitute organ is not yet complete or because of a defect in the growth or development of the substitute organ. Accordingly, vibration properties of a substitute organ may be used (alone or in combination with other properties as described herein) to determine the stage of development of the substitute organ and/or to evaluate whether it is ready for transplantation or other applications.

In some embodiments, vibrations of a tissue may result from the tissue response to forces such as blood flow, air flow, etc., or any combination thereof. In some embodiments, physiological forces in a subject may cause natural vibrations of tissue or organ structures in the body. In some embodiments, organs grown ex vivo (e.g., in a bioreactor) may vibrate naturally in response to mechanical forces associated with growth in the bioreactor (e.g., fluid pumped through a vasculature, or gas pumped in and out of airways, etc.).

Natural vibrations may be detected using any suitable technique, including for example, optical techniques. In some embodiments, a laser may be used to interrogate a target region on a tissue or organ and the reflected wave energy may be evaluated to determine the vibration properties of that region. In some embodiments, the surface properties of an organ or other tissue may be evaluated. However, in some embodiments, internal properties of an organ or other tissue also may be evaluated by selecting an interrogating laser frequency and/or energy that is sufficient to penetrate to a depth of interest and provide a reflected signal that can be evaluated. For example, wavelengths from 600 to 3000 nm may be used in the IR range. These wavelengths maybe used to detect surface movement or vibrations by measuring the vibrations deflection by the response of the reflected light. In some embodiments, heat vibrations may indicate vibrational patterns. In some embodiments, visible light may be used if the subject tissue is exposed. In some embodiments, IR may be used for exposed tissue and/or through tissue to make non-invasive measurements.

It should be appreciated that the resolution of the analysis may be determined by the wavelength of the interrogating laser. In some embodiments, a millimeter scale resolution may be used. However, a centimeter scale resolution also may be used since changes in vibration properties at the centimeter scale may be sufficiently informative for diagnostic and/or therapeutic applications and/or for evaluating a tissue or organ in a reactor (accordingly, one or more energy transfer devices may be located within a reactor chamber and manipulated so that they can be applied to an organ or tissue surface and used for evaluation. It should be appreciated that other resolution scales may be used as aspects of the invention are not limited in this respect.

In some embodiments, a 3-dimensional evaluation may be obtained by using a plurality of interrogating laser waves arranged in a suitable configuration. In some embodiments, an array of interrogating laser waves may be used. In some embodiments, the interrogating laser may be directed onto an organ or tissue that is surgically exposed in a subject or that is grown in a bioreactor. However, in some embodiments, an energy transfer device (e.g., an optical port) as described herein may be used in order to transmit the interrogating laser and/or receive the resulting signal. In some embodiments, a plurality of laser-transparent members may be arranged in an array on a single support member of an energy transfer device and/or a plurality of energy transfer devices may be used in order to obtain 3-dimensional information from a target organ or tissue region of interest.

It should be appreciated that the results of the analysis (e.g., the vibrational properties or the elasticity of the tissue or organ) may be displayed using any suitable technique. In some embodiments, different thresholds may be set and different levels of vibration (e.g., different vibration amplitudes) may be represented using different colors and/or intensities. In some embodiments, the vibration display may be overlaid with one or more different displays (e.g., visual images, reconstructed images, heat profiles, etc., or any combination thereof) to provide additional functionality or information. In some embodiments, certain combinations of vibration and other properties (e.g., heat) may be used for diagnostic purposes. For example, an abnormal vibration profile in combination with an abnormal heat profile may identify a organ or tissue region as diseased or injured with greater statistical significance than either profile alone.

In some embodiments, a vibration display may be overlaid with a visual display of an organ to assist in a surgical procedure. For example, a display of abnormal vibration in an infracted heart may be overlaid with a display of the heart in order to target a therapy (e.g., a cellular injection, for example, using a stem cell or other multipotent cell preparation) to one or more damaged regions of the heart that are abnormal due to dead or dying cells caused by insufficient oxygenation.

In some embodiments, the vibration of the organ or tissue may be observed using a head-mounted device as described herein. In some embodiments, the head-mounted device is used to detect and analyze energy that was introduced using an energy transferring device as described herein to assist in transferring an interrogating laser wave (or array of laser waves) to one or more regions of a target tissue or organ of interest.

It should be appreciated that aspects of the invention may be used in combination with any suitable surgical procedure or intervention where target tissue may be identified based on abnormal vibration, heat, or other profiles, or any combination thereof. In some embodiments, needle or surgical instrument of interest may be directly observed or may include a tag (e.g., an RFID or other suitable tag) that allows the instrument (or the operating end of the instrument) to be precisely located on the image display (e.g., on the overlay of the vibration profile, visual image, and any other suitable profile such as a heat profile). This allows the surgeon to target an injector tip (e.g., needle) or other surgical tool to a precise tissue area that was identified as damaged based on an abnormal vibration profile, heat profile, other physical profile, or a combination of two or more thereof.

In some embodiments, an abnormal organ or portion thereof may be replaced using a substitute organ or portion thereof that was grown in a bioreactor. Aspects of the invention may be used to assist in the transplantation or implantation procedure to identify the appropriate target regions in a recipient patient.

In some embodiments, an overlay of a vibration profile and a visual display of a region of interest may be used directly for diagnostic purposes and/or therapeutic intervention. However, in certain embodiments, a region of abnormal vibration may be identified and located in a tissue or organ using a standard reference frame (e.g., having i) a standard origin relative to defined structural properties of the tissue or organ, and ii) standard axes and units) as described herein.

In some embodiments, a normal and/or diseased profile may be defined in comparison to a known normal profile. The known normal profile may be a standard reference profile for a normal tissue or organ. In some embodiments, a subject may be scanned to obtain a personalized reference for one or more healthy organs and or tissues (provided the organs or tissues are healthy in the subject at the time of the reference analysis). This healthy reference may be stored as part of the patient medical records used for comparison to profiles obtained during subsequent evaluations. Changes in vibration profiles, heat profiles, other physical properties, or any combination thereof, at one or more locations within a tissue or organ may be used to identify diseased regions or may be used as an initial screen to identify tissue or organs that need to be evaluated using additional techniques in order to determine their status.

In some embodiments, a normal and/or diseased profile may be defined in comparison to a known diseased profile.

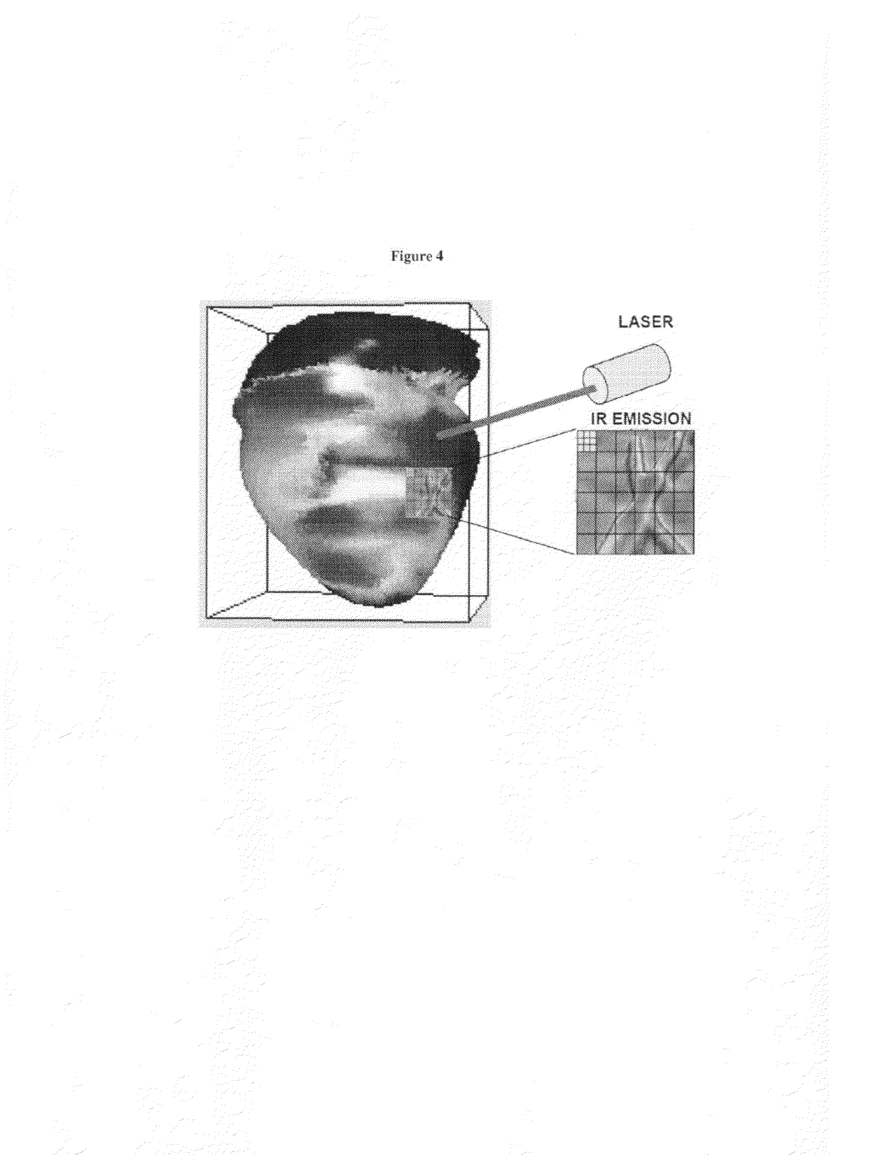

FIG. 4 illustrates a non-limiting example of a heart that is being evaluated to identify its pattern of spatial vibrational and heat distributions to determine whether normal patterns have been disrupted (which could be indicative of an infracted heart, for example). This analysis may be performed on an organ in a patient in order to identify and/or target potential abnormalities. This analysis also may be performed on a substitute organ grown in a bioreactor to evaluate its properties and determine whether it is suitable for transplantation (e.g., by comparison to a reference substitute heart profile known to be suitable for transplantation).

In some embodiments, in addition or as an alternative to measuring natural vibration frequencies of an organ or tissue, one or more external physical and/or chemical stimuli may be applied in order to measure the vibration profile of a target region in response to the stimuli.

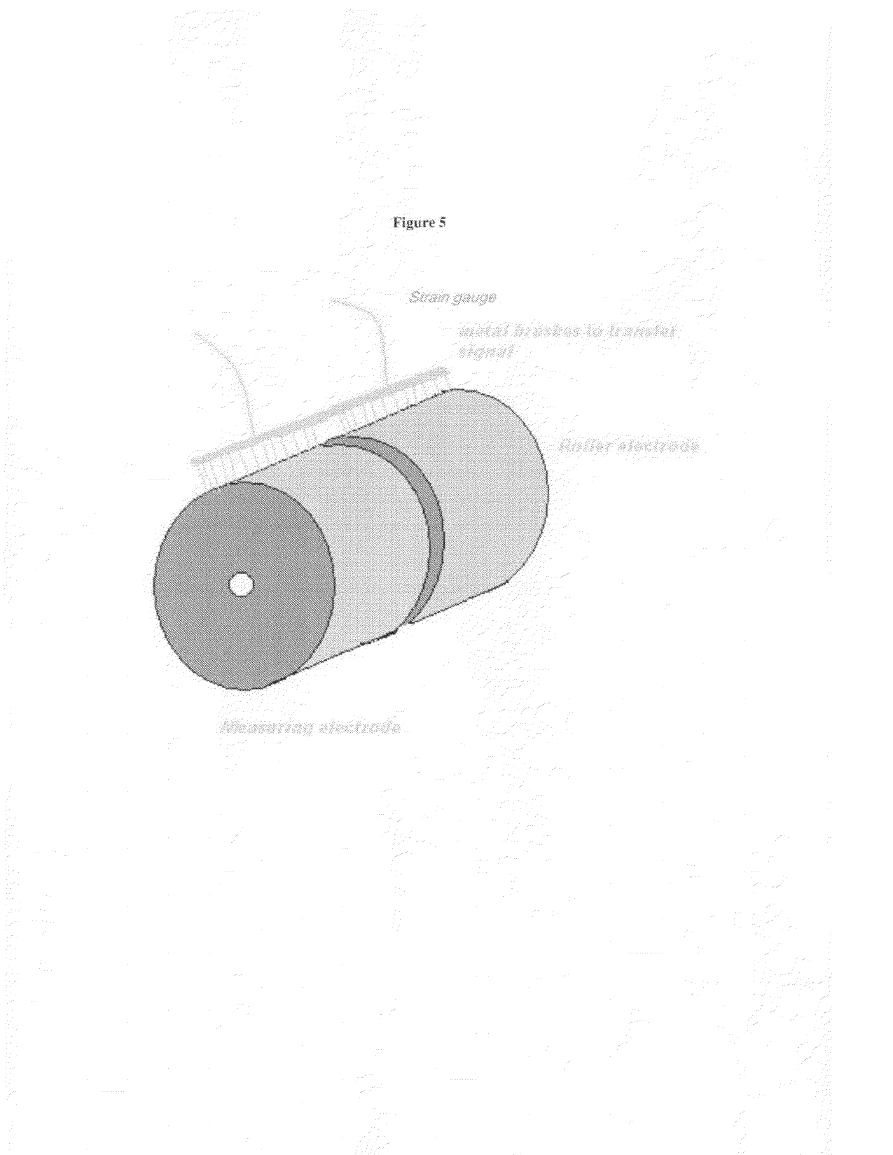

In some embodiments, aspects of the invention relate to methods and devices for measuring electrical signals from tissues or organs. In some embodiments, an electrode may include a conductive rolling member at its measuring end. The rolling electrode end can be applied to the surface of a tissue or organ and is useful to measure a signal in response to pressure exerted by the rolling member on the tissue. An advantage of the rolling member is that pressure can be exerted with minimal damage to the tissue, unlike a standard electrode that includes one or more sharp tips. The applied pressure can be used to provide and maintain a good electrical contact between the tissue and the electrode and/or to physically stimulate tissue or organ surface and measure the response to the stimulus. The rolling member may be a cylinder, ball, or other shape that can be rolled across the surface of a tissue or organ. FIG. 5 illustrates a non-limiting example of a cylindrical rolling member 50. An axis 52 around which the rolling member rotates may be connected to a support structure (not shown) on the electrode. However, any suitable configuration for providing a rolling tip may be used. In some embodiments, the rolling member may rotate around two or more axes to provide greater freedom of movement in operation. Electrical contact between the rolling member and the remainder of the electrode may be maintained using one or more metal brushes 54 as illustrated in FIG. 5. However, it should be appreciated that other electrical connections may be used as aspects of the invention are not limited in this respect. In some embodiments, the electrode also includes a strain gauge 56 to measure the force exerted by the electrode on to the surface of the tissue or organ. In some embodiments, the strain gauge may be connected to a controller that regulates the amount of pressure that the electrode exerts on the surface.

It should be appreciated that the rolling member includes conductive material (e.g., a metal, conductive ceramic, glass, conductive polymer, etc., or any combination thereof) on its surface. In some embodiments, the rolling member is connected to an electrode arm that may be connected to one or more robotic motors that control the motion of the electrode on the tissue. However, in some embodiments, a hand-held measuring electrode including a rolling member may be used.

It should be appreciated that an electrode may include an array of rolling members, all of which may be connected to the same processor and/or display unit to analyze and/or represent the electrical signals measured by the rolling member(s) in any suitable format. In some embodiments, only abnormal signals are displayed.

In some embodiments, a representation of the electrical profile of an organ or tissue surface may be overlaid in a display (e.g., a head-mounted display) along with a visual display and/or one or more of a heat profile (e.g., IR profile), vibration profile, and/or other physical profile as described herein. Accordingly, electrical profiles obtained from one or more electrodes described herein may be used to monitor or target a surgical intervention as described herein in connection with other information.

In some embodiments, probes may include pressure sensors. In some embodiments, elasticity and pressure waves may be sensed through and on a surface (e.g., of a tissue or organ). In some embodiments, a probe also may have a light sensor (e.g., to detect light in the IR range, for example, from 600-3000 nm). In some embodiments, a probe may be able to detect or include filters that are adapted for oxygen-sensing (e.g., wavelength around 500 nm) or for non-oxygen-sensing (e.g., wavelength around 700 nm).

It should be appreciated that one or more of the probes or electrodes described herein may be located within a reactor and manipulated to evaluate an organ or tissue (e.g., using a robotic manipulator or other device within the reactor that can be controlled from outside the reactor). Accordingly, the probes and/or electrodes can be sterilized (e.g., made of sterilizable material). It should be appreciated that any suitable sterilization technique may be used for any of the reactor components, probes, sensors, solutions, etc., provided an appropriate technique is selected to avoid damage to the item being sterilized. Suitable techniques for different applications may include filtration, heat inactivation, chemical or enzymatic sterilization, radiation, or any other technique or any combination thereof.

In Situ Tissue or Organ Growth and Transplantation:

In some embodiments, aspects of the invention relate to growing a substitute organ in situ in a patient or subject in order to provide natural body stimuli that may promote optimal growth. In some embodiments, a substitute organ is grown in a reactor that is implanted in a subject to provide appropriate internal stimuli. In some embodiments, organ growth may be initiated in a biodegradable envelope or support that can be transplanted. In some embodiments, a device may allow for monitoring organ growth in situ (e.g., for monitoring size and/or function). In some embodiments, one or more optical sensors may be used (e.g., infrared, nephelometry, etc.).

Accordingly, in some embodiments an implantable device may include a growth chamber that is open/accessible to in situ tissue. In some configurations the chamber may be open. However, in some configurations the chamber may be separated from in situ tissue by a membrane such as a permeable membrane. In some embodiments, a membrane may be a biodegradable and/or resorbable membrane. In some embodiments, an implantable device may include a fiber-optic bundle. In some embodiments, an implantable device may include a flexible chamber wall. In some embodiments, an implantable device may include a porous chamber wall to allow a growing substitute organ and host organ(s) to connect. In some embodiments, a device is adapted to be removed from the body of the recipient. In some embodiments, a device is adapted to be resorbed into the body of the recipient. In some embodiments, the device can measure the size and/or one or more functions of the substitute organ in situ. Accordingly, a device may include an optical sensor (e.g., IR, nephelometry, etc.).

In some embodiments, aspects of the invention include methods of growing and monitoring organs within a recipient. For example, a method may include implanting a device and removing it when organ has reached a threshold size or function; implanting a device made of a biocompatible material; and/or implanting a device made of a biodegradable/resorbable material.

In some embodiments, aspects of the invention relate to systems that include an implantable growth chamber and associated sensors, monitors, controllers, etc., all of which may be portable.

Growing an Organ Adapted for Transplantation:

In some embodiments, aspects of the invention relate to growing an organ so that is adapted or optimized for transplantation. In some embodiments, a substitute organ may be produced so that it includes additional tissue or tissues flaps or tabs that can be used to attach organ to recipient (e.g., via suturing or adhesion). In some embodiments, a substitute organ may have surplus length of vascular and/or neuronal tissue for easier connection to a recipient. In some embodiments, a substitute organ may have a size and/or shape that is adapted to the recipient's anatomy.

In some embodiments, a device or scaffold/matrix may have a shape that includes support for additional tissue or one or more flaps or tabs; may include support for a surplus length of vasculature; and/or may be adjustable to allow support for organs of different size and shape. In some embodiments, a decellularized scaffold may be reshaped and/or extended to be adapted for the recipient anatomy, and/or to include support for tabs or flaps for surgical attachment.

Accordingly, in some embodiments aspects of the invention relate to designing and growing organs with surplus tissue and/or vasculature for transplantation; transplanting organs using engineered tabs or flaps for attachment to a recipient; measuring particular recipient features and growing an organ to be compatible with those features (including, for example, volume, maximum length/height/width, location of vasculature, etc.).

Monitoring and Recording Growth Information:

In some embodiments, aspects of the invention relate to obtaining and maintaining data to provide records of events during organ growth (e.g., growth conditions, changes in growth conditions, etc.). Such information may be useful for regulatory compliance, to confirm that a substitute organ was exposed to appropriate conditions during growth, to identify anomalies during growth, etc., or any combination thereof. In some embodiments, aspects of the invention relate to recording and/or storing analytical information about actual conditions and functions of organ during growth.

In some embodiments, methods and devices may be provided for continuous physiological monitoring, removing samples for analysis, imaging applications, etc., or any combination thereof. In some embodiments, a bioreactor may be equipped to automatically record one or more specified events (e.g., amounts and timing of delivery of growth medium, additives, factors, gases, temperature changes, pressure, etc.). In some embodiments, a bioreactor may include one or more structures and/or mechanisms for sampling access; one or more windows for optical analysis; one or more sensors (e.g., optical, chemical, physical, etc., or any combination thereof); one or more specific sensors for material that is being introduced during growth to provide a confirmation or quality control information; or any combination thereof. In some embodiments, a method or system of the invention may be used for gathering or tracking data on events that occurred during growth, data on measurements, and/or combining data on events and measurements. It should be appreciated that the information may be used to confirm that appropriate growth conditions were maintained during growth of the substitute organ (e.g., appropriate growth environment, appropriate sterility, etc., or any combination thereof).

Flow Pattern Monitoring:

In some embodiments, flow patterns in a bioreactor, or in portions thereof, or within an organ in a bioreactor, or portions thereof (e.g., a vessel, a segment of an organ, etc.) may be used to evaluate the status of growth and/or development within the bioreactor. In some embodiments, infrared (IR) detectors may be used to monitor flow patterns. In some embodiments, a solution having a different temperature than the temperature within the bioreactor may be perfused into the system (e.g., into an organ or portion thereof) and flow patterns and/or changes in temperature can be used to evaluate metabolic activity, physiological function, or any combination thereof. In some embodiments, flow patterns can be used to evaluate the function of the bioreactor itself (e.g., to determine flow patterns for oxygenation or cell seeding, to identify or confirm regions of laminar or turbulent flow, etc.). In some embodiments, flow patterns within an organ can be used to evaluate the function of the organ and whether it is ready for transplantation (e.g., based on liquid or gas flow patterns to evaluate blood flow or respiratory properties, for example).

It should be appreciated that different flow patterns may be selected for different parts of a reactor system and/or for different stages of tissue or organ development. In some embodiments, a turbulent flow may be provided to prevent cells from being deposited at particular sites or within conduits. However, in some embodiments, minimal or no flow may be used at sites (e.g., a matrix or other support site) at which cells are deposited. In some embodiments, intermittent or periodic mixing may be used to allow cells to settle and attach without mixing and then be mixed to resuspend cells that are not attached and then allow them to settle again. This process may be repeated as often and for as long as necessary. It should be appreciated that any suitable mixing technique may be used (e.g., active or static mixers) and may be provided in conduits or manifolds to provide static baffles or other structures or movable elements, or any combination thereof that cause flow to be turbulent (and, e.g., to keep cells in suspension and/or to keep other material mixed). In the context of cell deposition on a scaffold or support structure, mixing may be achieved by inverting, shaking, or other physical manipulation of the support structure and/or of the chamber within which the support structure is located. It should be appreciated that other mixing techniques also may be used.

Cellular and Subcellular Evaluation:

In some embodiments, IR may be used to visualize and/or evaluate the confluence of cells and/or their metabolic activity in bioreactors. An analysis may be based on spectral illumination from NIR to FIR. In all cases an IR temperature and/or IR spectral selectivity may be used. It should be appreciated that every spectral wavelength can act as a non invasive dye specific to vibrations of different species of molecules as well as for detecting bond twisting. These properties can be used to image features in tissues, solutions or gases.

It should be appreciated that in some embodiments, molecular imaging may be used to monitor and/or evaluate the status of an organ, tissue, cell, or cellular organelle. In some embodiments, techniques such as atomic force (ATF) microscopy may be used. In some embodiments, IR physical probes (e.g., crystals or IR imaging on the molecular and atomic level) may be used. Molecular information may be used to indicate and/or evaluate metabolic activity. In some embodiments, an FTIR microscope may be used to detect or evaluate cellular or subcellular thermodynamic processes.

3D Tissue Analysis:

In some embodiments, aspects of the invention provide techniques for spatial orientation relative to an organ that changes size and in which cells change location during growth. It should be appreciated that the growth of an organ or tissue is three dimensional and non linear across the xyz plane of the entire tissue or organ. For example, a cell in a first position will move in an xyz direction that may be different from a cell in a second position in a different area of the tissue. In some embodiments, it is helpful to provide a reference for the original position, so that the growth and development of the organ or tissue can be evaluated. In some embodiments, scans (e.g., autoscans) at one or more time points (e.g., predetermined time points) may be used to track the movement (e.g., movement of one or more cells, or overall tissue movement) in an area or volume of interest. It should be appreciated that the time points (e.g., intervals between scans) may be selected so that the growth or cellular movement between scans is not too great to prevent tracking (e.g., so great that the organ or tissue at a second time point cannot be recognized relative to the organ or tissue at a first time), or so that the amount of growth does not move the organ or tissue out of the original field of view. In some embodiments, for each spot in memory the movement of that segment of tissue or organ or cells can be measured or calculated. In some embodiments, the direction and/or extent of change can be used (e.g., compared to other information) to provide a database of growth and development information and/or to monitor and/or predict normal or abnormal growth or development. In some embodiments, lasers may be used in a system to lock onto features in the tissues and organs that can be tracked. The movement of the laser could represent the movement of the fixed point along different x, y, and z axes in the tissue. This can be used to provide information about and/or evaluate the xyz movement and growth of tissues and organs. This can be fed into a computer to predict where original cell areas move to and how they develop. In some embodiments, these observations can be used to monitor and evaluate the confluence and non confluence of tissues and organs and also to help distinguish healthy from non-healthy tissues and organs. Accordingly, in some embodiments, one or both of the rate and/or direction of particular cellular or tissue movement during growth or development (e.g., on a scaffold or matrix) may be used to monitor and/or evaluate the stage of growth and/or the appropriateness of the growth. In some embodiments, inappropriate growth patterns may be detected early using this technique and either be corrected or be used as a basis for terminating the particular organ or tissue growth if the abnormal pattern is associated with (e.g., suspected of, or previously shown to be associated with) unacceptable organ or tissue growth that cannot be corrected.

The movement of cells and tissue during growth can be used to evaluate cellular confluence, rate of growth, normal and abnormal growth trends, patterns that are indicative of healthy organs suitable for transplantation, patterns that are indicative of unhealthy organs that are not suitable for transplantation, patterns that are indicative of organs that are growing appropriately but not yet ready for transplantation. It should be appreciated that any of the above parameters (e.g., cellular confluence, or other parameter) may be used either i) as a cue or marker for the level of organ development at any particular time (e.g., with reference to a database of normal or abnormal information for that time point) or ii) as a marker that an organ is ready for storage and/or transplantation.

In some embodiments, predetermined time and spatial references are selected and used to monitor and evaluate organ growth.

It should be appreciated that cell or organ tracking techniques may be used in other 2D or 3D contexts, for example for live cell imaging, for controlling microscope stage driver systems to find (e.g., return to) particular spots of interest (e.g., certain cellular regions of interest) while accommodating for the slippage of the mechanics and heat and cold variables, motor power differences, etc., or any combination thereof.

Sampling Configurations:

In some embodiments, a bioreactor or container (e.g., a container such as a bag containing cells, blood, drugs, nutrients, etc., or any combination thereof) may include a sampling volume that is isolated from the volume of the bioreactor or container by walls and a flow valve (e.g., a one way flow valve). In some embodiments, a sampling volume is not connected to the main volume of the bioreactor chamber or other container.

A sampling volume can be used for sample retrieval for further analysis. Alternatively or additionally, a sampling volume may include one or more sensors. These configurations may be used to analyze a sample isolated from a container or bioreactor without disturbing the remainder of the contents of the container or bioreactor. In some embodiments, a statistical sampling area can be monitored using methods which could interfere with (e.g., damage or destroy) the contents of a container or bioreactor.

In some embodiments, a sampling volume may be bounded by a wall that includes at least one appropriately transparent window suitable for a spectrometric measurement. In some embodiments, one or more sensors may be embedded in the material of a wall that surrounds a sampling volume. It should be appreciated, that one or more probes may be provided to measure creatine, glucose, O2, Co2, pH, temperature, lactic acid, and/or other nutrient or waste material, and/or other parameters described herein.

Storage and Shipping Containers:

In some embodiments, aspects of the invention relate to shipping and storage containers for organs. A shipping container may be temperature-regulated (e.g., cooled and/or heated and/or both to maintain a predetermined temperature setting). In some embodiments, a shipping container may be configured to maintain a temperature (e.g., about 4° C., 4-10° C., 10-20° C., or other temperature) suitable for storing or shipping an organ.

In some embodiments, a shipping container may be a thin container and/or have a thin wall (e.g., a metal container) that can be used to efficiently conduct heat to a cooling reservoir or sink (for example as used for liquid nitrogen containers to get good heat conduction) to maintain a selected temperature efficiently. The container may be insulated, for example using one or more configurations of insulating material around the contain (e.g., using about 1-5 inches, for example about 4 inches deep insulation all around). In some embodiments, a slot or other opening in the insulating material may be used to place the container in (or remove it from) the insulating material. In some embodiments, one or more Peltier devices (or other temperature control devices) may be attached on one or more sides of the container (e.g., the metal container). In some embodiments, a power source (e.g., a battery) may be located on top of the container (e.g., to avoid heat from the power source rising into the chamber of the container). However, it should be appreciated that other cooling techniques may be used as aspects of the invention are not limited in this respect. In some embodiments, one or more refrigeration units may be used (e.g., using a cylinder of compressed gas). In some embodiments, endothermic chemical reactions, may be used to provide cooling.

In some embodiments, a tissue or organ in a shipping container may be connected to one or more detectors and/or inputs and/or outputs (e.g., connected to reservoirs, filtration units, controllers, etc.) to allow the condition of the tissue/organ to be monitored and to allow conditions within the reactor to be controlled. In some embodiments, a shipping container may include a recording component suitable for recording the temperature of the container (e.g., for later analysis to confirm that appropriate temperatures were maintained during shipping and/or storage of an organ).

It should be appreciated that in some embodiments, an organ or tissue may be shipped in a portion of the original reactor that remains after removal of reactor zones and/or components associated with one or more prior phases (e.g., decellularization, recellularization, and/or growth). In some embodiments, the remaining reactor zone after removal of the other modules may have one or more features adapted for transport and/or storage as described herein.

Computer-Related Implementations:

Aspects of the methods disclosed herein may be implemented in any of numerous ways. For example, the various methods or processes outlined herein may be coded as software that is executable on one or more processors that employ any one of a variety of operating systems or platforms. Such software may be written using any of a number of suitable programming languages and/or programming or scripting tools, and also may be compiled as executable machine language code or intermediate code that is executed on a framework or virtual machine. Information described herein relating to cells, organs, tissue, growth conditions, reference parameters for growth and development, etc., or any combination thereof may be encoded and/or stored on a database and used as described herein. Relationships between the different types of information may be encoded to allow for efficient use as described herein.

Accordingly, in some embodiments, measurements of one or more parameters described herein may be related or compared to a database of normal and abnormal values for the different parameters, taken alone, or in combination (e.g., of 2, 3, 4, 5, 6, 7, 8, 9, 10 or more). The resulting determination (e.g., that measurements are similar or different, for example with statistical significance) may be a basis for modifying a growth condition, keeping or rejecting an organ or tissue (for example at an early stage during development without needing to wait for full development), or identifying an organ as being ready for storage, transport, or surgery.

In this respect, aspects of the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed herein. The computer readable medium or media can be transportable, such that the program or programs stored thereon can be loaded onto one or more different computers or other processors to implement various aspects of the present invention as discussed above.

For example, in some embodiments, information regarding the development, status, or health of a cell, tissue or organ may be recorded on a computer readable medium together with one or more programs that when executed on one or more computers or other processors, perform methods that evaluate the status and/or health of the cell, tissue or organ during growth; direct and/or optimize growth of the cell, tissue or organ; determine and/or establish appropriate growth conditions for the cell, tissue or organ; evaluate whether the cell, tissue or organ is growing normally (e.g., is healthy or physiologically acceptable) and/or is growing abnormally (e.g., shows signs of inappropriate growth or function); or determine when the cell, tissue or organ is ready for transplantation. Various types of information may be recorded on a computer readable medium, including, for example, spatial, physiological, metabolic, mechanical, chemical, histological, electrical, and/or structural inter-relationships between and among cells, tissues, and organs. Reference information regarding the development of a cell, tissue or organ may also be recorded on the computer readable medium, including, for example, normal values for parameters (mechanical, histological, chemical, etc.) relating to growth of a cell, tissue or organ. Information may also include images (infrared, visible, fluorescence, etc.) depicting the developmental state or health of a cell, tissue or organ. In some embodiments, the computer readable medium may include, recorded thereon, one or more of the following types of information: species of a cell, tissue or organ, cell type, tissue type, organ type, scaffold type, date of tissue, source of cells, source of tissue, source of organ, temperatures of incubation, infrared, fluorescence and/or visible confluence images, O₂, CO₂, pH, lactate, glucose, creatine, start date, projected end date, target implantation site, age of subject, health of subject, etc., or any combination thereof.

The terms “program” or “software” are used herein in a generic sense to refer to any type of computer code or set of computer-executable instructions that can be employed to program a computer or other processor to implement various aspects of the present invention as discussed herein. Additionally, it should be appreciated that according to one aspect of this embodiment, one or more computer programs, which when executed perform certain methods disclosed herein, need not reside on a single computer or processor, but may be distributed in a modular fashion among or between a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Power Sources:

In some embodiments, a bioreactor or biological container described herein may be powered by a hybrid power system (e.g., using two, three, or more different power sources). In some embodiments, a power source may be based on one or more conventional heating or cooling masses (e.g., based on chemical heating or cooling reactions), solar conversion of light to energy, and/or the conversion of ambient heat or cold to power. Solar and/or electrical power (e.g., USB, 485, 110/220, Battery, motion, ambient temperature to power converters, etc.) may be used alone or in combination. It should be appreciated that chemical reactions, cold packs, refrigerant, high velocity gas supply temperature, may be used for temperature regulation (e.g., heating or cooling) without being power sources (e.g., they may be used as cooling and/or heating electron donors).

In some embodiments, a container (e.g., a storage, transport, bioreactor container) may include an insulated environment that can maintain its internal temperature against ambient temperature changes (e.g., it can hold the temperature in the contain with minimal loss to the outside). Regardless of the temperature setting for the container (e.g., 4° C., 23° C. or other suitable temperature), the container may have a heating or cooling source to maintain a stable temperature, and also an active temperature regulator to control the amount of heating or cooling to maintain the set temperature. It should be appreciated that the regulator may require a power source. It must react (e.g., to internal temperature changes) to deliver additional heating or cooling as necessary to maintain the set internal temperature. In some embodiments, hybrid power sources may be used to increase the portability of a temperature-regulated container and also to provide temperature regulation without adding the bulk of batteries or without the need for a companion (e.g., human intervention) to monitor or interact with power (and for example a temperature regulator). In some embodiments, a container can maintain its temperature without requiring an external power source. However, it should be appreciated that a container may be connected to an external power source (e.g., plugged into an outlet) in order to provide temperature regulation. In some embodiments, a temperature-regulated container may included both batteries and a secondary power source in order to maintain the life of the batteries. In some embodiments, a secondary power source may be any suitable electrical outlet (e.g., a cigarette lighter or other outlet in a vehicle, a USB connection from a computer or laptop, a electrical outlet in a building, or other electrical source). Accordingly, a temperature-regulated container may include a connector having one or more adaptors for different power sources. A secondary power source also may be provided by solar power, or any suitable energy converting material that can be used to produce more energy for the batteries. In some embodiments, a container may include a solar panel, a panel of material that converts heat to electricity, or other power generating source. One or more of these can be used to provide sufficient additional power to heat or cool a container and/or to regulate sources of heating or cooling provided along with the container (e.g., to maintain a predetermined temperature of 4° C., 23° C., or other desired temperature).

In some embodiments, thermoelectric materials that can convert heat into electricity and electricity into heat, may be used. In some embodiments, silicon nanowire-based converters may be used. In some embodiments, “rough” silicon nanowires may be used. For example, suitable material may be created in a process of “electroless etching” in which arrays of silicon nanowires are synthesized in an aqueous solution on the surfaces of wafers. This technique results in vertically aligned silicon nanowires having exceptionally rough surfaces and surprisingly high thermoelectric efficiency.

Accordingly, in some embodiments, a container may be provided with more than one power source and/or more than one heating and/or cooling sources.

In some embodiments, a hand-activated power source (e.g., a crank or other mechanical power source connected, e.g., one that induces current into the system) also may be attached or connected to a storage and/or transport device.

In some embodiments, a transport and/or storage device may include one or more signals (e.g., optical, audible, or other signal) that indicates when additional power is needed (e.g., to maintain the set temperature, and/or to maintain one or more of the monitors and/or life support systems).

In some embodiments, a container may be disposable (e.g., a disposable bag) and contain one or more sensors. A hybrid power source may consist of batteries and light (e.g., electrical and/or solar light) driven power (e.g., so that any suitable natural or electrical light source may be used). In some embodiments, the light-dependent power source may be used to provide a trickle charge to the batteries.

Printers for Compositions Comprising Cells

In some aspects of the invention, printers are provided for printing support structures with our without cells. In some embodiments, a printer may be used to deposit scaffold material in an appropriate 2D or 3D configuration for a support structure described herein. In some embodiments, the printers may be used to print cells onto a scaffold or other support structure. For example, cells may be printed on an in vitro substrate, such as, for example, a cover slip surface, cell culture plate or well bottom, an artificial or isolated extracellular matrix, a natural or synthetic scaffold, etc. In some embodiments, cells may be printed on a biological tissue, which may either be an isolated tissue or an in vivo tissue. For example, cells may be printed directly on an isolated tissue, e.g., a dermal tissue. In another example, cells may be printed directly on a wound (e.g., a burn, an ulcer, infarction, etc.) to provide cells (e.g., stem cells, skin cells, etc.) for repairing the wound.

In some embodiments, printers for printing biological materials or cells are provided. The printer typically comprises a print head and one or more motors or devices for moving the print head to control deposition of the composition onto a substrate. The print head is typically designed and configured to translate and/or rotate along or about one or more axes. In some cases, the print head may be designed and configured to move in three-dimensional space with 1, 2, 3, 4, 5 or 6 degrees of freedom. Accordingly, the print head may be designed and configured to move forward-backward, up-down, and/or left-right (translation in three perpendicular axes). In some embodiments, the print head is designed and configured to rotate about one, two, or three perpendicular axes (i.e., pitch, yaw, roll).

Typically the print head is designed and configured to house a composition to be printed. In some embodiments, the print head comprises a removable print cartridge that houses a composition to be printed. The print head is often designed and configured to have one or more temperature control elements that heat and/or cool the composition to maintain material or cells at a predetermined temperature. In some embodiments, the temperature control elements include a heating and/or cooling element. In some embodiments, the temperature control element includes a thermocouple to measure the temperature in the cartridge. In some embodiments, the temperature control elements are designed and configured to maintain a temperature in a range of 0° C. to 10° C., 5° C. to 20° C., 10° C. to 40° C., 20° C. to 50° C., 4° C. to 37° C. or 0° C. to 50° C. In some embodiments, the temperature control elements are designed and configured to maintain a temperature of up to 4° C., 10° C., 20° C., 30° C., 40° C., 50° C. or more.

The print head is also typically designed and configured to maintain any of a variety of other parameters important for cell homeostasis, including, for example, O₂ saturation, pH, nutrient concentration, etc. The print head typically comprises one or more fluid conduits for adding and/or removing fluids, e.g., for adding a buffer, for perfusing a gas, e.g., CO₂, O₂, etc.

In some embodiments, the printer cartridge may include one or more components for heating, cooling, oxygenating, detoxifying, filtering, and/or monitoring or otherwise regulating the composition (e.g., material with or without cells) it contains.

In some embodiments, the print head may be designed and configured to release the composition with or without cells onto a substrate in a controlled manner. In some embodiments, the print head controls the volume of the composition that is deposited and/or the relative location at which the composition is deposited. The print head may be fluidically connected with one or more pumps, e.g., one or more pumps that create a pressure gradient sufficient to expel the composition from the print head. In some embodiments, the print head is designed and configured to spray droplets of the composition comprising cells onto a substrate. Thus, in some embodiments, the printer functions similar to an inkjet printer that sprays droplets of ink. In some embodiments the print head has a face plate with a plurality of nozzles. In some embodiments, each nozzle has an outlet in a range of 0.05 to 200 μm in diameter, 1 to 100 μm in diameter, 5 to 200 μm in diameter, or 10 to 50 μm in diameter. A plurality of nozzles with the same or different diameters may be provided in some embodiments. Though in some embodiments the nozzles have a circular opening, other suitable shapes may be used, e.g., oval, square, rectangle, etc., taking into account the relative size of the cells intended to be deposited.

In some embodiments, a printer comprises one or more devices or components for particle filtration, O₂ adjustment, CO₂ maintenance, pH adjustment, nutritional adjustments, waste product removal, etc. In some embodiments, these devices or components are integrated into or coupled with the printer head, e.g., intergrated into a printer cartridge. In some embodiments, the printers serves as an injecting device, defrosting device, and/or cell preparation device. In some embodiments, the printers are designed and configured to maintain the metabolic, anatomical, and/or physiological integrity of cells, thus ensuring cells are viable and functionally active following printing.

In some embodiments, printers may be designed and configured to print a biopolymer or inorganic polymer to create printed organs and/or tissues. In some embodiments, printers may be designed and configured to print a combination of biological cells and a biopolymer or inorganic polymer to create printed organs and/or tissues.

Materials:

It should be appreciated that components of the bioreactors and other devices or containers described herein may be manufactured any suitable rigid or flexible material, for example using metal, glass, rubber, plastic, composite, other natural or synthetic material, or any combination thereof. Where polymeric materials are used, such materials can be selected or formulated to have suitable physical/mechanical characteristics, for example, by tailoring the amounts of components of polymer blends, adjusting the degree of cross-linking (if any), etc. For instance, those of ordinary skill in the art can choose suitable polymers for use in bioreactors based on factors such as the polymer's compatibility with certain processing techniques, compatibility with any materials contained in the container (e.g., cells, nutrients, gases, etc.), compatibility with any treatments or pre-treatments (e.g., sterilization, autoclaving), flexibility, puncture strength, tensile strength, liquid and gas permeabilities, and opacity.

Optionally, a vessel/chamber and/or bioreactor, or components thereof, may be transparent to certain wavelengths of light (e.g., to visible light, ultraviolet light, X-rays, etc.) to allow viewing and/or monitoring of contents inside the vessel. In certain embodiments, a vessel and/or bioreactor, or components thereof, is compatible with certain medical imaging techniques such as magnetic resonance imaging (MM), fluoroscopy, computed tomography (CT), positron emission tomography (PET), thermography, and ultrasound. For instance, non-paramagnetic materials may be used for certain components when MRI is contemplated. Advantageously, such compatibility can allow detection of conditions or processes involving of cells, tissue(s), and/or organ(s) in the bioreactor, while maintaining sterility of the cells, tissue(s), and/or organ(s) contained in the bioreactor.

In some embodiments, a component is USP Class VI certified, e.g., silicone, polycarbonate, polyethylene, and/or polypropylene. Non-limiting examples of polymers that can be used to form a component include polyethylene (e.g., linear low density polyethylene and ultra low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. Components may comprise a substantially rigid material such as a rigid polymer (e.g., high density polyethylene), metal, and/or glass.

Bioreactors or components thereof may be sterilized using any suitable technique prior to use.

In some embodiments, one or more components or zones of a bioreactor (e.g., a chamber) may be removable from one or more (or all) connecting conduits, pumps, wires, detectors, support mechanisms in a way that maintains sterility of the chamber. It should be appreciated that any suitable fluid connectors (e.g., with sealable valves, plugs, or other mechanisms of maintaining sterility when a conduit is disconnected), electrical connectors (e.g., electrical plugs, sockets or other electrical connectors), and/or mechanical connectors (e.g., sockets, tabs, screws, clips, etc.), or any combination thereof may be used.

In some embodiments, a surface of one or more components of a reactor (e.g., a growth chamber, a conduit, a pump, a storage chamber, a support structure, a mechanical manipulator such as a robotic arm) may be coated with one or more compounds to promote sterility (e.g., an antimicrobial compound), prevent adhesion (e.g., expanded polytetrafluoroethylene or ePTFE/Teflon, or related or other material), prevent clotting (e.g., heparin), promote cell or tissue bonding, or other compounds (e.g., a coating that amplifies or blocks a signal, for example, an IR signal, a coating that blocks UV or other forms of radiation, depending on the application), or any combination thereof, depending on the structure that is being coated. Anti-reflection coatings may be used for some applications. Examples of anti-reflection coatings include, but are not limited to, IR anti-reflection coatings, for example, but not limited to, zinc sulfide, zinc selenide, gallium arsenide, germanium, silicon. CaF2, BaF2, IR fused Silica, and Saphire. However, other single layer or multiple layer anti-reflection (e.g., IR or UV anti-reflection) coatings may be used.

It should be appreciated that the inner surfaces of hollow components (e.g., conduits, chambers, etc.) may be coated whereas the outer surfaces of other structures that are within the chamber or other parts of a reactor system (e.g., support structures, manipulators, flow mixers—static or active, etc.) may be coated. In some embodiments, an anti-adhesion coating may be used on surfaces that come into contact with flow of a perfusion material (saline, artificial blood, or other perfusate). In some embodiments, an adhesion promoting material may be used in connection with support structures and or matrices that are intended to attach to cells or tissue. It should be appreciated that a coating may be permanent or semi-permanent and may be attached or deposited using any suitable technique. It also should be appreciated that a coating may be applied in any suitable pattern depending on the application and the regions that are helpful to have coated.

Therapeutic or Agent Delivery:

In some embodiments, one or more device components may have at least one portion that is transparent in the 700 nm to 1,000 nm range so that IR radiation can be used to target a support (e.g., on a solid or in an encapsulated form, for example associated with a nanoparticle, a polymer, a matrix, a scaffold, or one more regions of the bioreactor) to promote the release of a therapeutic or other agent at a time of interest. Similarly, one or more alternative sources of energy (e.g., electro-magnetic, micro-wave, or other wavelengths or forms of radiation) may be used to release a therapeutic or other agent at a time and a place of interest (e.g., one a support as described above). Examples of therapeutic agents include, but are not limited to heparin, other anti-coagulant, an agent that reduces restenosis (e.g., paclitaxel), or other agent that may be useful in maintaining or promoting appropriate growth or physiological conditions).

It should be appreciated that the examples described in the Figures and Examples relate to specific embodiments, and the related description is not-limiting to all embodiments described herein. However, it should be appreciated that structures, methods, compositions, devices, related components and technical steps and other aspects described in the context of the examples provided in the Figures and Examples can be used in combination with other embodiments and applications described herein.

The following examples are intended to illustrate certain embodiments of the present invention, but are not to be construed as limiting and do not exemplify the full scope of the invention.

### EXAMPLES

### Example 1

### Organ Identity and Patient Matching

In some embodiments, fluorescence quenching (or FRET) may be used to evaluate DNA complementarity for organ identity and/or matching. Since only an exact match would bind to its complimentary single strand, simply heating the combined DNA samples to denature them with one side (e.g., the patient's) being labeled with a fluorophore and the other side (e.g., the organ's) being labeled with a quencher would result in fluorescence extinction upon binding (which would only take place if there was a perfect match). This technique may need some sample preparation steps but one or more of these may be incorporated into a device integral to the chamber so the user (e.g., doctor or nurse) simply has to add the patient swab and the apparatus does everything else. In some embodiments, a device could automatically sample a small piece of tissue that is provided or grown inside the chamber to be used for an identity matching application.

In some embodiments, iontophoresis may be used to label a substitute organ and/or a recipient subject. Iontophoresis is a non-invasive method of propelling high concentrations of a charged substance, normally medication or bioactive-agents, transdermally by repulsive electromotive force using a small electrical charge applied to an iontophoretic chamber containing a similarly charged active agent and its vehicle. One or more chambers are filled with a solution containing an active ingredient and its solvent, termed the vehicle. The positively charged chamber, termed the anode will repel a positively charged chemical, whilst the negatively charged chamber, termed the cathode, will repel a negatively charged chemical into the skin. This technique may be used to label a substitute organ and/or a subject with one or more markers (e.g., dyes, nucleic acids, proteins, or other markers). However, other techniques may be used.

### Example 2

In some embodiments, multiple parallel processing units may be used to produce the entire organ or vessel being regenerated. In certain embodiments, a series of microscope slides with nano-sensors and liquid circuits may be organized and placed in any orientation, for example, in a bath (flexible or rigid). These nano-devices can hold one or more scaffolds. In some embodiments, the slides may have structural shapes conducive to the organ being regenerated. Accordingly, a scaffold, one or more sensors, and/or liquid flow all may be controlled by a parallel processing lab on a chip. Different components may be used to measure confluence, and/or pressure, and/or send signals.

In some embodiments, a cellular multi-level adhesion spindle may be used for regenerating tubular organs. In some embodiments, a spindle may be molded or machined with vacuum capabilities which pull down through raised platforms that have tunnels allowing the vacuum to suck down. The platforms can be at different levels. In some embodiments, a vascular substrate may be intertwined with the tubes. Small cells are passed onto the spindle first, and they stick to vacuum holes in the tubes. Big cells are provided second and get caught by big tubes. The spindle is in middle layer. It should be appreciated that other configurations may be used. For example, the vacuum aspect may be replaced by cups or other receptacles that are first filled by large cells. Smaller cells then are layered on the larger cells. This technique may be used to provide a stable, multilevel cell differentiated starter for a tubular organ.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A bioreactor comprising,
a chamber configured for containing an organ under culture conditions,
a first inlet port and a first outlet port in fluid communication with the chamber, and
an organ support structure disposed in the chamber.

2. The bioreactor of claim 1, wherein the organ support structure is connected to a scale or gauge for measuring the weight of the organ; or wherein the organ support structure comprises a platform, optionally wherein the platform is shaped to receive the lower portion of an organ; or wherein the organ support structure comprises a first support member, optionally wherein the first support member comprises a hook; or wherein the organ support structure comprises a first tubular connector adapted for attaching a vascular structure.

3-7. (canceled)

8. The bioreactor of claim 2, wherein the first tubular connector is cylindrical; or wherein the first tubular connector comprises a flexible flange; or wherein the first tubular connector comprises an elastic flange; or wherein the first tubular connector comprises an expandable flange, optionally wherein the expandable flange is remotely controlled; or wherein the first tubular connector comprises a tapered end; or wherein the first tubular connector comprises a flared end.

9-14. (canceled)

15. The bioreactor of claim 1, wherein the organ support structure is in a fixed position relative to the chamber; or wherein the organ support structure can rotate around a first axis that is in a fixed position relative to the chamber; or wherein the organ support structure can rotate around a second axis that is in a fixed position relative to the chamber.

16-17. (canceled)

18. The bioreactor of claim 1, further comprising one or more sensors responsive to pressure, flow, pO2, pH, CO2, lactate, glucose, electrical, ion concentration, mechanical force, torque, stretch, fluorescence, emissivity, vibrational properties and/or response to external energy, and/or temperature.

19-24. (canceled)

25. The bioreactor of claim 1, wherein the chamber wall comprises a sterile access port; or wherein the chamber wall comprises an observation area that is transparent to infrared, UV, and/or visible light; or wherein the chamber wall comprises a translucent portion, optionally wherein the translucent portion comprises polysulfone or any other sterilizable material that is transparent to infrared and/or visible wavelengths; or wherein the chamber wall comprises a flexible portion, optionally wherein the flexible portion can be used to limit the distance of the fluid barrier between an organ and a detector to less than about 3 mm; or wherein the chamber wall comprises a section of elastic material.

26-35. (canceled)

36. The bioreactor of claim 1, further comprising a first stimulatory means,
optionally wherein the first stimulatory means can administer an electrical challenge to a substitute organ attached to the organ support structure, and optionally further comprising a sensor capable of detecting a response to the electrical challenge; or
optionally wherein the stimulatory means can administer a chemical challenge to a substitute organ attached to the organ support structure, and optionally further comprising a sensor capable of detecting a response to the chemical challenge; or
optionally wherein the stimulatory means can administer a physical or mechanical challenge to a substitute organ attached to the organ support structure, and optionally further comprising a sensor capable of detecting a response to the physical challenge.

37-44. (canceled)

45. The bioreactor of claim 36, wherein the challenge represents a physiological parameter selected from the group consisting of blood pressure, pH, oxygen, toxin, metabolite, airflow, substrate, force, torque, hormone, and any combination thereof; or wherein the response is a level of a physiological parameter selected from the group consisting of blood pressure, pH, oxygen, toxin, metabolite, and any combination thereof that can be measured using any mechanical, optical, and/or chemical sensor.

46. (canceled)

47. The bioreactor of claim 1, further comprising a 2-dimensional or 3-dimensional array of sensors to determine the size, shape, weight, tensile strength, blood vessel strength, or strength of attachment to a substrate of a substitute organ attached to the organ support structure, wherein the sensors can detect any mechanical, optical, and/or chemical signal.

48-53. (canceled)

54. The bioreactor of claim 1, further comprising a substitute organ attached to the organ support structure, optionally including a sensor capable of detecting a response to an electrical challenge, a chemical challenge or a physical challenge; or, further comprising a scaffold attached to the organ support structure, optionally wherein the scaffold is a decellularized organ scaffold.

55-56. (canceled)

57. The bioreactor of claim 54, wherein the substitute organ is a substitute solid or hollow organ, optionally wherein the substitute solid or hollow organ is a substitute lung, liver, kidney, heart, or pancreas.

58-61. (canceled)

62. The bioreactor of claim 1, further comprising a first tag for identifying, tracking, or confirming the origin of a substitute organ attached to the organ support structure, optionally wherein the first tag is an electronic tag, a magnetic tag, an RFID tag, a barcode, or any combination thereof.

63. (canceled)

64. The bioreactor of claim 1, further comprising a means for removing cells from the chamber to identify, track, or confirm the origin of a substitute organ attached to the organ support structure.

65. The bioreactor of claim 1, further comprising an injector for injecting material into a substitute organ attached to the organ support structure.

66. The bioreactor of claim 1, further comprising a biopsy device for removing material from a substitute organ attached to the organ support structure.

67. A system comprising a bioreactor of claim 1 connected to a pump via one or more conduits, wherein each of the chamber, pump, and one or more conduits are of material that is compatible with use with an MRI, CAT, PET, X-ray analysis, or ultrasound device.

68. A system of claim 67, wherein the material of each of the chamber, pump, and one or more conduits is non-metallic, and/or non-paramagnetic.

69-71. (canceled)

72. A kit comprising a first tag to be attached to an organ recipient and a second tag to be attached to a bioreactor or system of claim 67 or to a substitute organ within said bioreactor.

73. (canceled)

74. The kit of claim 72, wherein the first and second tags are independently selected from an electronic tag, a magnetic tag, and RFID tag, a barcode, or any combination thereof.

75-79. (canceled)

80. The bioreactor of claim 1, wherein the chamber has a volume of between 20 cc and 20,000 cc, between 500 cc and 1,000 cc, between 1,000 cc and 10,000 cc, between 10,000 cc and 20,000 cc, or larger.

81-338. (canceled)
